(19) United States
(12) Patent Application Publication
(10) Pub. No.: US 2008/0064098 A1
(43) Pub. Date: Mar. 13, 2008

(54) Procurement, isolation and cryopreservation of maternal placental cells

(75) Inventor: Julie G. Allickson, Odessa, FL (US)

Correspondence Address: JOHN W. GOLDSCHMIDT, JR. ESQUIRE;DILWORTH PAXON LLP, 3200 MELLON BANK CENTER, 1735 MARKET STREET, PHILADELPHIA, PA, 19103, US

(73) Assignee: Cryo-Cell International, Inc., Oldsmar, FL (US)

(21) Appl. No.: 11/810,932

(22) Filed: Jun. 5, 2007

Related U.S. Application Data
(60) Provisional application No. 60/811,156, filed on Jun. 5, 2006.
(60) Provisional application No. 60/811,651, filed on Jun. 6, 2006.
(60) Provisional application No. 60/811,935, filed on Jun. 7, 2006.
(60) Provisional application No. 60/876,591, filed on Dec. 22, 2006.

(51) Int. Cl. C12N 5/08; C12M 3/00

(52) U.S. Cl. 435/366; 435/307.1; 435/308.1; 435/374; 435/378

(57) ABSTRACT

Methods, processes and systems for procuring, isolating and cryopreserving at least one viable, multipotent maternal placental stem cell is provided. Viable maternal placental stem cells are also provided. The maternal placental stem cell of the invention expresses the cell surface marker CD117 and at least one of the cell surface markers selected from the group consisting of CD29, CD44, CD73, CD90, CD105, CD166, SSEA-3 and SSEA-4 and has low or no expression of at least one of the cell surface markers selected from the group consisting of CD34, CD45, CD133, TRA-1-60 and TRA-1-81. The methods and process comprise generally obtaining a piece of placental tissue from a whole placenta, disaggregating the placental tissue with mechanical separation or enzymatic digestion, collecting and concentrating placental cells comprising maternal placental cells with centrifugation and optionally cryopreserving the placental cells. Additional steps for selecting and culturing the maternal placental stem cells is provided.

### CROSS REFERENCE TO RELATED APPLICATIONS

This application claims the priorities of U.S. Provisional Patent Application Ser. No. 60/811,156, filed Jun. 5, 2006; U.S. Provisional Patent Application Ser. No. 60/811,651, filed Jun. 6, 2006; U.S. Provisional Patent Application Ser. No. 60/811,935, filed Jun. 7, 2006; and U.S. Provisional Patent Application Ser. No. 60/876,591, filed Dec. 22, 2006, each entitled “Procurement, Isolation and Cryopreservation of Placental Stem Cells,” the entireties of which are incorporated herein by reference.

### FIELD OF THE INVENTION

The invention relates to methods, processes, and systems for procuring and processing placental tissue obtained from a whole placenta; for isolating, collecting and cryopreserving a population of placental cells obtained from the procured placental tissue; and for selecting, culturing and cryopreserving viable, multipotent maternal placental cells.

### BACKGROUND OF THE INVENTION

The human placenta develops from cells of fetal and maternal origin during implantation of a fetus into a uterus. Placental tissue is a combination of fetal cells that form fetal placental tissue and maternal cells that form maternal placental tissue. The fetal tissue and maternal tissue are affixed together with connective tissue to form the diversely functional placenta. Many of the fetal cells and the maternal cells of the placenta are characteristically capable of proliferation and differentiation. The fetal cells present in the placenta include, but are not limited to, fetal stem cells, hematopoietic cells, epithelial cells, mesenchymal or fibroblast-like cells, trophoblast cells, and other progenitor cells. The maternal cells present in the placenta include but are not limited to maternal mesenchymal or fibroblast-like cells, maternal-embryonic-like stem cells, cells of the maternal immune system and other maternal cells.

Maternal cells and fetal cells coexist in the placenta throughout gestation. In comparison to fetal cells, there is only limited information on maternal cells of the placenta. The fetal portion of a term placenta comprises the umbilical cord, the amniotic membrane on the cord, the amnion and the chorionic plate. During maturation of the placenta, the chorionic villi fuse with the amnion forming a seamless layer of amniochorion. The maternal portion comprises maternal decidual tissues on the opposite side of the umbilical cord and, also, the maternal blood that flows through the capillaries in the chorion. Maternal cells have been retrieved from a collection of maternal decidual tissue. Int'Anker P S et. al in 2004 harvested tissue from the decidua basalis and decidua parietalis, but did not reliably demonstrate the presence of maternal mesenchymal cells when subjected to functional viability assays from term placentae (Int'Anker P S et al. Stem Cells 2004; 22:1338-1345). Takahashi et al in 2002 (International Application Patent Number: PCT/JP03/03760) discussed the harvest and differentiation of maternal mesenchymal stem cells from placenta. The tissue was again harvested specifically from the maternal side of the organ and cells were isolated by explant or enzyme digest. These mesenchymal stem cells were unable to be differentiated into adipogenic or chondrogenic cell lineages.

Stem cells inherently possess the capability to undergo cellular division and cellular differentiation in vivo by way of control of cell-to-cell contact and intrinsic signals. Stem cells have been shown to be capable of dividing and differentiating in vitro into a variety of cells by controlling cell contact and intrinsic signals by stimulation with local environmental factors. It is recognized that stem cells may be obtained from several sources including a variety of adult tissues, such as bone marrow and also embryonic tissues.

The transplantation of adult stem cells derived from bone marrow has been successfully used in treatment of human disease such as Fanconi's Anemia, Aplastic Anemia, Acute and Chronic Leukemias, Myeloproliferative Disorders, Myelodysplastic Syndromes, Lymphoproliferative Disorders and other malignancies. Alternative sources of bone marrow adult stem cells include peripheral blood progenitor cells, umbilical cord blood and mesenchymal stem cells harvested from these sources. However, there are several shortcomings associated with therapeutic use of adult stem cells. Adult stem cells have been shown to have limited efficacy such as slow growth and loss of pluripotency after several passages in culture.

Embryonic stem cells have demonstrated proliferative potential making them suitable for cellular therapy. However, human embryonic stem cells have been shown to produce teratomas. Additionally, the harvesting of stem cells from embryos poses ethical concerns due in part to the destruction of the growing embryo in the harvesting process.

Other sources of stem cells or cells having stem cell-like characteristics have been identified that overcome at least some of the issues associated with adult and embryonic stem cells.

One source is umbilical cord blood that contains human adult stem cells. Umbilical cord blood has proven to be a viable source of stem cells for several reasons. Umbilical cord blood is relatively easy to procure during delivery of a child and to process for cryopreservation. Umbilical cord blood provides a suitable source of stem cells capable of cellular division and differentiation into a variety of cell types. Stem cells derived from cord blood have been used in clinical settings to treat several known human disorders and diseases as an alternative to bone marrow. In particular, stem cells derived from cord blood have been successful in hematopoietic engraftment and hematopoietic reconstitution. A case study demonstrates the use of umbilical cord blood in treating spinal cord injuries. Additionally, stem cells derived from cord blood have been shown in animal models to reverse the effects of stroke and myocardial infarction. Further research is being conducted to identify additional therapeutic uses of stem cells derived from cord blood.

Advancements in research in the area of human stem cells have led researchers to alternative sources of stem cells including chorionic villi from the placenta and amniotic fluid cells. Collection of chorionic villi tissue and amniotic fluid for potential use in cell therapy involves the practice of methodologies that overcome the issues associated with collecting embryonic stem cells. Amniotic fluid containing fetal stem cells can be collected through the practice of amniocentesis. Amniocentesis involves the insertion of a fine needle through the abdomen of a pregnant woman into the uterus and amniotic sac. The needle is used to withdraw a desired amount of amniotic fluid containing human fetal stem cells from within the amniotic sac. In addition, chorionic villi are composed of cells that are of fetal origin and include fetal stem cells that can be collected through the practice of transcervical or transabdominal chorionic villi sampling (CVS). Chorionic villi are finger-like projections that emerge from the chorionic plate of the placenta and form part of the fetal portion of the placenta. Research indicates that selected human stem cells derived from amniotic fluid or chorionic villi are pluripotent and are, therefore, capable of differentiating into all three germ cell layers, highly proliferative, lacking significant immunogenicity due to no expression of HLA class II, and positive for the antigenic factor CD117 as described in United States Patent Application Publication No. US 2005/0124003.

Another source of viable stem cells is the human placenta post childbirth. Research indicates that fetal stem cells and other progenitor cells, including those cells from the maternal tissue, are present in the placenta. These stem cells and progenitor cells may be useful for cellular therapy or tissue engineering. Research indicates that cells obtained from the placenta may be capable of differentiating along osteogenic, adipogenic, chondrogenic, myogenic, endothelial, hepatic, neurologic and hematopoietic cell lineages. Other studies have examined the use of human umbilical cord tissue as a source of stem cells.

Use of a human placenta as a source of fetal stem cells and other maternal cells is beneficial for several reasons. The placenta is considered biohazardous medical waste after the birth of a baby. The placenta is typically discarded so the placenta is easily accessible for procurement of fetal and maternal cells from the placental tissue. Additionally, the process of procuring placental tissue after birth imparts no risk to the donor or the baby as may be associated with other fetal tissue collection techniques such as amniocentesis and CVS. Furthermore, any ethical concerns or considerations are generally alleviated by collecting placental tissue that is otherwise considered biohazardous medical waste.

One method for collecting placental stem cells is set forth in U.S. Pat. No. 7,045,148 to Hariri. The method appears to comprise collecting embryonic-like stem cells from a placenta, which has been treated to remove residual cord blood, by perfusing the drained placenta with an anticoagulant solution to flush out residual cells, collecting the residual cells and perfusion liquid from the drained placenta, and separating the embryonic-like cells expressing the cell marker CD34 from the residual cells and perfusion liquid. In order to practice the method, an exsanguinated placenta having the proximal umbilical cord clamped is recovered and transported to a laboratory for processing. At the laboratory, the umbilical cord is cannulated and connected to a perfusion manifold that pumps perfusion solution into the placenta. During the perfusion step, the placenta is used as a bioreactor for residual and stem cells present in the placenta, which are flushed out of the maternal side of the placenta and collected with effluent. Stem cells expressing the CD34 cell marker are isolated from the other residual cells and the effluent. The method requires shipment of a whole, intact placenta to the facility for processing so that the cells present within the vasculature of the placenta may be flushed out of the vasculature through the maternal side of the placenta.

Certain methods for isolating, expanding and differentiating fetal stem cells from chorionic villus, amniotic fluid and the placenta and related therapeutic uses are set forth in United States Patent Application Publication No. US 2005/0124003 to Atala et al. Generally, methods for collecting a population of c-kit positive cells are set forth and comprise collecting a piece of chorionic villi or amniotic fluid during pregnancy or, alternatively, a sample of placenta after birth and processing the tissue or fluid to collect c-kit positive cells. The c-kit positive cells sought in the practice of the method express embryonic stage specific cell markers Stage Specific Embryonic Antigen-3 (SSEA-3) and Stage Specific Embryonic Antigen-4 (SSEA-4). In particular, the methods described focus on isolating and culturing a heterogeneous population of cells collected from amniotic fluid, chorionic villi and placenta and selecting the c-kit positive cells by flow cytometry, gradient magnetic selection, and implementation of a solid phase.

Typically, placental cell procurement involves harvesting cells or placental tissue through procedures such as amniocentesis or CVS at a health care facility or, alternatively, through procedures such as transporting whole placentae to a laboratory for processing. In the latter of the two procedures, a laboratory facility generally receives and processes an entire placenta, which requires implementation of resources to collect, ship and process a whole placenta. Additionally, there are a variety of target cells which are purported to be obtained through the methodologies in the prior art, e.g., CD34 or c-kit cells. However, there is no teaching for procuring a suitable size of a piece of human placenta at the bedside post-delivery, and related tissue processing and cell collection and culturing methodologies useful for obtaining viable maternal placental cells expressing at least one of the cell surface markers CD44 and CD117 and not expressing CD45. The maternal cells may also express at least one of the cell surface markers CD29, CD73, CD90, CD105, CD166, SSEA-3 and SSEA-4 and may have low or no expression of at least one of the cell surface markers CD34 and CD133.

The methods of the present invention may be used to collect maternal cells from tissue of the placenta or alternatively the umbilical cord. The maternal cells include viable maternal placental stem cells also referred to herein as maternal-placental stem cells (“MPSC”).

Accordingly, there is a present need for methods to collect a suitable piece of tissue from a human placenta or, optionally, the umbilical cord, at the bedside, for ready shipment to a centralized laboratory for processing to isolate and to store maternal cells of the placenta. There is also a present need for methods to process placental tissue to obtain a cell preparation comprising viable maternal placental stem cells from the maternal portion of the placental tissue. There is also a further need to select, culture, isolate and cryopreserve viable maternal placental cells from the population of cells such as, for example, maternal placental stem cells that express at least one of the cell surface markers CD44 and CD117 and do not express the cell surface marker CD45. The maternal placental stem cells also may express at least one of the cell surface markers selected from the group consisting of CD29, CD73, CD90, CD105, CD166, SSEA-3 and SSEA-4 and may have low or no expression of at least one of the cell surface markers CD34 and CD133. There is yet a further need to cryopreserve the population of cells or the isolated maternal placental stem cells obtained from the pieces of placenta with the processes, methods and systems of the present invention.

### SUMMARY OF THE INVENTION

The present invention provides methods, processes and systems for procuring a piece of placental tissue from a whole placenta upon delivery or cesarean section at the bedside for ready packaging and shipment to a processing facility. The present invention also provides methods, processes and systems for processing a piece of placental tissue comprising maternal placental tissue to isolate, collect, concentrate, and preserve a population of cells comprising maternal placental stem cells expressing at least the cell surface marker CD117. Optionally, a piece of umbilical cord tissue may be processed in place of the placental tissue in accordance with the present invention.

The present invention provides further methods, processes and systems for isolating, culturing and selecting maternal placental stem cells expressing at least one of the cell surface markers CD44 and CD117 and not expressing the cell surface marker CD45. The maternal placental stem cells may also express at least one of the cell surface markers CD29, CD73, CD90, CD105, CD166, SSEA-3 and SSEA-4 and may have low or no expression of at least one of the cell surface markers CD34 and CD133. The present invention also provides methods for cryopreserving a population of placental cells and maternal placental stem cells.

A population of placental cells comprising viable maternal placental stem cells expressing CD117 is provided by the present invention. Additionally, an enriched population of viable maternal placental stem cells expressing at least one of the cell surface markers CD44 and CD117 and not expressing the cell marker CD45 is provided. The maternal placental stem cells may also express at least one of the cell surface markers CD29, CD73, CD90, CD105, CD166, SSEA-3 and SSEA-4 and may also have low or no expression of at least one of the cell surface markers CD34, CD133, TRA-1-60 and TRA-1-81. Moreover, a composition of the population of cells comprising maternal placental stem cells expressing CD117 is provided. Furthermore, a composition of maternal placental stem cells expressing the cell surface markers CD44 and CD117 and not expressing the cell surface marker CD45 is provided by the present invention.

The methods, processes and systems may be used to procure and process maternal placental stem cells obtained from maternal tissue harvested from a placenta or umbilical cord to produce a yield of viable, multipotent maternal placental cells enriched in a population of placental cells that may be cultured or cryopreserved.

A method for obtaining a population of placental cells comprising viable maternal placental stem cells expressing the cell surface marker CD117 and at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45, CD133, TRA-1-60 and TRA-1-81 from placental tissue comprising maternal placental tissue is provided by the present invention. The method comprises the steps of isolating placental cells by disaggregating the placental tissue and separating the placental cells from disaggregated placental tissue by either mechanical separation or enzymatic digestion; collecting the placental cells as a population of placental cells and concentrating the population of placental cells comprising maternal placental cells with at least one step of centrifugation; and cryopreserving the population of placental cells comprising maternal placental cells at a temperature at or below about −135° C.

A process for obtaining a population of placental cells comprising viable maternal placental stem cells expressing the cell surface marker CD117 and at least one of the cell surface markers selected from the group consisting of CD29, CD44, CD73, CD90, CD105, CD166, SSEA-3 and SSEA-4 and having low or no expression of at least one of the cell markers selected from the group consisting of CD34, CD45, CD133, TRA-1-60 and TRA-1-81 from placental tissue comprising maternal placental tissue is provided by the present invention. The process comprises the steps of procuring placental tissue comprising maternal placental tissue and fetal placental tissue obtained from a whole placenta; optionally removing a portion of the fetal placental tissue from the placental tissue; disaggregating the placental tissue by either mechanical separation or enzymatic digestion; isolating a population of cells from disaggregated maternal placental tissue comprising maternal placental cells; and collecting the population of cells comprising maternal placental stem cells by concentrating the population of placental cells with at least one step of centrifugation.

A process for collecting viable maternal placental stem cells expressing CD117 and at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45, CD133, TRA-1-60 and TRA-1-81 from placental tissue obtained from a whole placenta is provided by the present invention. The process comprises the steps of isolating placental cells comprising maternal placental stem cells from the placental tissue by disaggregating the placental tissue; collecting and concentrating the placental cells; and cryopreserving the placental cells.

The present invention provides a population of cells comprising maternal placental stem cells expressing CD 117 and at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45, CD133, TRA-1-60 and TRA-1-81.

A system for collecting a population of cells comprising maternal placental stem cells expressing CD117 and at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45, CD133, TRA-1-60 and TRA-1-81 from placental tissue comprising maternal tissue is provided by the present invention. The system comprises a placental cell isolater, a placental cell collector, placental cell concentrator, and a placental cell cryopreserver. The placental cell isolator disaggregates placental tissue comprising maternal tissue and separates placental cells from the disaggregate placental tissue. The placental cell collector collects the placental cells separated from the disaggregate placental tissue. The placental cell concentrator concentrates placental cells present in a suspension. The placental cell cryopreserver maintains the collected and concentrated placental cells at a temperature at or below about −135° C.

A population of viable maternal placental stem cells expressing CD117 obtained from a process of the present invention. The population of viable maternal placental stem cells is obtained by the process comprising culturing a population of placental cells comprising maternal placental stem cells; and selecting placental cells expressing CD117 from a culture of the population of placental cells to obtain the population of viable maternal placental stem cells expressing CD117.

A population of viable maternal placental stem cells expressing CD117 obtained from a process of the present invention. The population of maternal placental cells is obtained by the process comprising the steps of selecting placental cells expressing CD117 from a culture of the population of placental cells to obtain the population of viable maternal placental stem cells expressing the cell surface marker CD117.

A population of maternal placental stem cells expressing CD117 is obtained from a process of the present invention. The process comprising the steps of selecting placental cells expressing CD117 from a population of placental cells comprising maternal placental cells to obtain the population of maternal placental stem cells expressing CD117; culturing the placental cells expressing CD117 selected from the population of placental cells comprising maternal placental stem cells; and selecting placental cells expressing CD117 from a culture of placental cells expressing CD117 comprising a population of viable maternal placental stem cells.

A process for isolating a population of viable maternal placental stem cells expressing CD117 from a population of placental cells is provided by the present invention. The process comprising the steps of culturing a population of placental cells comprising maternal placental stem cells; selecting placental cells expressing CD117 from a culture of the population of placental cells; and cryopreserving the maternal placental stem cells expressing CD117.

A process for isolating a population of viable maternal placental stem cells from a population of placental cells is provided by the present invention. The process comprising selecting placental cells expressing CD117 from a culture of the population of placental cells.

A process for isolating a population of maternal placental stem cells expressing CD117 from a population of placental cells is provided by the present invention. The process comprising the steps of selecting placental cells expressing CD117 from a population of placental cells comprising maternal placental cells; culturing the placental cells expressing CD117 selected from the population of placental cells comprising maternal placental stem cells; and selecting placental cells expressing CD117 from a culture of placental cells.

The present invention provides a process for obtaining a population of cells enriched for maternal placental stem cells expressing CD117. The process comprises the steps of culturing a population of placental cells obtained from placental tissue comprising at least maternal placental tissue and selecting CD117 cells from the cell culture whereby the selected cells may also express at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45, CD133, TRA-1-60 and TRA-1-81.

A population of cells enriched for maternal placental stem cells obtained from a process of the present invention is provided. The process comprises culturing a population of cells comprising maternal placental stem cells and selecting cells expressing CD117 from a culture of the population of cells. The population of cells express at least one of the cell surface markers selected from the group consisting of CD29, CD44, CD73, CD90, CD105, CD117, CD166, SSEA-3 and SSEA-4 and having low or no expression of at least one of the cell surface markers selected from the group consisting of CD34, CD45, CD133, TRA-1-60 and TRA-1-81.

Maternal placental stem cells are obtained from a process of the present invention. The process comprising the steps of selecting placental cells expressing CD117 from a population of placental cells comprising maternal placental stem cells, culturing the placental cells expressing CD117 selected from the population of cells comprising maternal placental stem cells, and selecting placental cells expressing CD117 from a culture of placental cells expressing CD117.

At least one maternal placental stem cell obtained by a process of the present invention is provided. The process comprising the steps of procuring placental maternal tissue and fetal tissue from a whole placenta; disaggregating the placental tissue; isolating placental cells comprising maternal placental stem cells from disaggregated placental tissue; collecting and concentrating placental cells comprising maternal placental stem cells in a population of cells; culturing the population of placental cells comprising maternal placental stem cells; and selecting at least one maternal placental stem cell expressing CD117 from a culture of the population of placental cells.

The present invention comprises a process for enriching a population of placental cells for maternal placental stem cells expressing CD117. The process comprises the steps of selecting CD117+ cells from a population of placental cells obtained from placental tissue, and cryopreserving the cells selected from the cell culture, whereby the selected cells may also express at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45, CD133, TRA-1-60 and TRA-1-81.

The present invention comprises a process for enriching a population of placental cells for maternal placental stem cells expressing CD117. The process comprises the steps of selecting CD117 cells from a population of placental cells obtained from placental tissue, culturing the CD117 cells selected from the population of placental cells, and selecting the CD117 cells from cell culture, whereby the selected cells may also express at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45, CD133, TRA-1-60 and TRA-1-81.

The present invention comprises a process for enriching a population of cells for maternal placental stem cells expressing CD117. The process comprises the steps of selecting CD117 cells from a population of placental cells obtained from placental tissue, culturing the CD117 cells selected from the population of placental cells, selecting the CD117 cells from cell culture, and culturing the selected CD117+ cells, whereby the selected cells may also express at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45, CD133, TRA-1-60 and TRA-1-81.

An enriched population of maternal placental stem cells obtained from maternal tissue of the placenta is provided as obtained and collected by the methods, processes or systems of the present invention. The enriched population of maternal placental stem cells express at least one of the surface cell markers selected from the group consisting of CD29, CD44, CD73, CD90, CD105, CD117, CD166, SSEA-3 and SSEA-4 and having low or no expression of at least one of the cell surface markers selected from the group consisting of CD34, CD45, CD133, TRA-1-60 and TRA-1-81. The enriched population of placental cells may be obtained by disaggregating placental tissue to release placental cells of the placental tissue; collecting and concentrating the placental cells released from the maternal; and culturing or cryopreserving an enriched population of maternal placental cells obtained from the maternal tissue of the placenta. Such population of cells enriched for maternal placental cells may be cryopreserved at a temperature at or below about −135° C.

The present invention provides an enriched population of placental cells comprising maternal placental stem cells expressing at least one of the cell surface markers selected from the group consisting of CD29, CD44, CD73, CD90, CD105, CD117, CD166, SSEA-3 and SSEA-4 and having low or no expression of at least one of the cell surface markers selected from the group consisting of CD34, CD45, CD133, TRA-1-60 and TRA-1-81.

The present invention provides an enriched population of maternal placental stem cells expressing the cell surface marker CD117 and at least one of the cell surface markers selected from the group consisting of CD29, CD44, CD73, CD90, CD105, CD166, SSEA-3 and SSEA-4.

The present invention provides a population of cells enriched for maternal placental stem cells expressing at least of the cell surface markers selected from the group consisting of CD29, CD44, CD73, CD90, CD105, CD117, CD166, SSEA-3 and SSEA-4 and having low or no expression of at least one of the cell surface markers selected from the group consisting of CD34, CD45, CD133, TRA-1-60 and TRA-1-81.

The present invention provides a composition of a population of cells comprising maternal placental stem cells expressing CD117 and a preservation agent.

The present invention provides a composition of at least one viable maternal placental stem cell expressing CD117 and a preservation agent.

The present invention provides a composition of at least one viable maternal placental stem cell expressing at least one of the cell surface markers selected from the group consisting of CD29, CD44, CD73, CD90, CD105, CD117, CD166, SSEA-3 and SSEA-4 and having low or no expression of at least one of the cell surface markers CD34, CD45, CD133, TRA-1-60 and TRA-1-81 and a preservation agent.

The present invention provides a composition of at least one viable maternal placental stem cell expressing CD29, CD44, CD73, CD90, CD105, CD117, CD166, SSEA-3 and SSEA-4 and having low or no expression of the cell surface markers CD34, CD45, CD133, TRA-1-60 and TRA-1-81.

The present invention provides a composition comprising at least one viable maternal placental stem cell and a cryopreservation agent.

The present invention provides a composition comprising at least one viable maternal placental stem cell and a preservative.

The present invention provides a composition comprising at least one viable maternal placental stem cell in a solution comprising a soluble protein.

A method is provided by the present invention for shipping at least one piece of placental tissue from a whole placenta. The method comprises the steps of obtaining at least one piece of placental tissue from a whole placenta, packaging the at least one piece of placental tissue to maintain the piece of placental tissue at about 1° C. to about 15° C. for shipment, and shipping the piece of placental tissue to a processing facility so that the at least one piece of placental tissue arrives at the processing facility within about 72 hours of delivery of the whole placenta.

### BRIEF DESCRIPTION OF THE FIGURES

FIG. 1 is an illustration of a view of the fetal side of a whole human placenta having a portion of the proximal umbilical cord attached and showing a scalpel cutting a piece of placental tissue in accordance with the invention.

FIG. 2 is an illustration of a sectional view of a whole human placenta as illustrated in FIG. 1.

FIG. 3 is an illustration of the piece of placenta shown in FIG. 1 procured with a scalpel.

FIG. 4 is a flow chart showing generally the overall process of the present invention.

FIG. 5 is a flow chart showing a process of the present invention generally illustrated in FIG. 4 where placental tissue is obtained by punch biopsy, placental tissue is disaggregated by enzymatic digestion of the placental tissue, placental cells are isolated from the placental tissue through cell separation by centrifugation, and the placental cells are collected and concentrated through centrifugation steps and optionally cryopreserved as a population of cells.

FIG. 6 is a flow chart showing another embodiment of the process generally illustrated in FIG. 4 where placental tissue is obtained by punch biopsy, placental tissue is disaggregated by enzymatic digestion of the placental tissue, placental cells are isolated from the placental tissue through cell separation by centrifugation, and placental cells are collected with a density gradient in a buffy coat layer that is collected and concentrated through centrifugation steps and optionally cryopreserved as a population of cells.

FIG. 7 is a flow chart showing an additional embodiment of the process generally illustrated in FIG. 4 where placental tissue is obtained by punch biopsy, placental tissue is disaggregated by mechanical separation of the placental tissue, the placental cells are isolated from the placental tissue through cell separation with a filter and wash, and the placental cells are collected and concentrated through centrifugation steps and optionally cryopreserved as a population of cells.

FIG. 8 is a flow chart showing a further embodiment of the process generally illustrated in FIG. 4 where placental tissue is obtained by punch biopsy, maternal placental tissue is disaggregated by mechanical separation of the placental tissue, placental cells are isolated from the placental tissue through cell separation with a filter and wash, and placental cells are collected with a density gradient in a buffy coat layer that is collected and concentrated through centrifugation steps and optionally cryopreserved as a population of cells.

FIG. 9 is a flow chart showing yet another embodiment of the process generally illustrated in FIG. 4 where placental tissue is obtained with scalpel and forceps, placental tissue is disaggregated by enzymatic digestion of the placental tissue, placental cells are isolated from the placental tissue through cell separation by centrifugation, and the placental cells are collected and concentrated through centrifugation steps and optionally cryopreserved as a population of cells.

FIG. 10 is a flow chart showing yet an additional embodiment of the process generally illustrated in FIG. 4 where placental tissue is obtained with scalpel and forceps, maternal placental tissue is disaggregated by enzymatic digestion of the placental tissue, placental cells are isolated from the placental tissue through cell separation by centrifugation, and placental cells are collected with a density gradient in a buffy coat layer that is collected and concentrated through centrifugation steps and optionally cryopreserved as a population of cells.

FIG. 11 is a flow chart showing yet a further embodiment of the process generally illustrated in FIG. 4 where placental tissue is obtained with scalpel and forceps, maternal placental tissue is disaggregated by mechanical separation of the placental tissue, placental cells are isolated from the placental tissue through cell separation with a filter and wash, and the placental cells are collected and concentrated through centrifugation steps and optionally cryopreserved.

FIG. 12 is a flow chart showing another alternative embodiment of the process generally illustrated in FIG. 4 where placental tissue is obtained with scalpel and forceps, maternal placental tissue is disaggregated by mechanical separation of the placental tissue, placental cells are isolated from the placental tissue through cell separation with a filter and wash, and placental cells are collected with a density gradient in a buffy coat layer that is collected and concentrated through centrifugation steps and optionally cryopreserved.

FIG. 13a is a flow chart showing an embodiment of the invention comprising selecting CD117 placental cells from a cell culture grown from a population of cells collected in accordance with any of the methodologies illustrated in FIGS. 4 through 12 and then optionally cryopreserving the selected CD117 maternal placental stem cells.

FIG. 13b is a flow chart showing an embodiment of the invention comprising selecting CD117 placental cells from a population of cells collected in accordance with any of the methodologies illustrated in FIGS. 4 through 12 and then optionally cryopreserving selected CD117 maternal placental stem cells.

FIG. 13c is a flow chart showing an embodiment of the invention comprising selecting CD117 placental cells from a population of cells collected in accordance with any of the methodologies illustrated in FIGS. 4 through 12, culturing the placental cells, selecting CD117 placental cells from culture and then optionally cryopreserving selected CD117 maternal placental stem cells.

FIG. 13d is a flow chart showing an embodiment of the invention comprising thawing a cryopreserved population of cells collected in accordance with any of the methodologies illustrated in FIGS. 4 through 12, culturing the cells, selecting CD117 placental cells from the culture and then optionally cryopreserving the selected CD117 maternal placental stem cells.

FIG. 13e is a flow chart showing an embodiment of the invention comprising thawing a cryopreserved population of cells collected in accordance with any of the methodologies illustrated in FIGS. 4 through 12, selecting CD117 placental cells from the culture and then optionally cryopreserving the selected CD117 maternal placental stem cells.

FIG. 13f is a flow chart showing an embodiment of the invention comprising thawing a cryopreserved population of cells collected in accordance with any of the methodologies illustrated in FIGS. 4 through 12, selecting CD117 placental cells, culturing the selected CD117 placental cells, selecting CD117 placental cells from the culture and then optionally cryopreserving the selected CD117 maternal placental stem cells.

FIG. 13g is a flow chart showing an embodiment of the invention comprising thawing a cryopreserved population of cells collected in accordance with any of the methodologies illustrated in FIGS. 4 through 12, culturing the cells, selecting CD117 placental cells from the culture, culturing the selected CD117 cells, and then optionally cryopreserving the cultured CD117 maternal placental stem cells.

FIG. 14 shows the results of the analysis for the TNC for the pre-processing sample, referenced in Example 1A, collected after disaggregation by mechanical separation and separation with a cell strainer, collection of placental cells by centrifugation at 2000 rpm for 7 minutes at about 15 to 30° C., and resuspension of pelleted placental cells in about 2 ml to about 3 ml of wash solution and combination of the cellular suspension with HBSS, whereby the results indicate the collection of 314.4 million placental cells.

FIG. 15 shows the results of the TNC analysis for the post-processing sample, referenced in Example 1A, collected as one ml of cellular suspension and placed in an analysis tube for flow cytometry analysis, whereby the results indicate the collection of 16.2 million placental cells.

FIGS. 16a through 16j show the representative results of flow cytometry analysis for cellular expression of CD44, CD45 and CD117 and viability of the cells in the post-processing sample of Example 1A. The post-processing sample was collected as one ml of cellular suspension and placed in an analysis tube for flow cytometry analysis. The results indicate a concentration of CD117⁺ cells at 0.4% of the TNC population, a concentration of CD117+ CD45− cells at 59.9% of the TNC population, a concentration of CD117+ CD44+ cells at 47.5% of the TNC population, and about 98% cell viability as determined by 7AAD. The results were calculated by running the post-processing sample in accordance with the flow cytometry analysis of the invention and subtracting background calculations obtained with a post-processing sample run with an isotype of an IgG antibody.

FIGS. 17a through 17e show representative flow cytometry results for the cells of Cell Line 23a, analyzed for cellular expression of CD44, CD45 and CD117 along with cell viability with 7AAD according to the methods of the present invention. Cell Line 23a was obtained from a placenta by the collection and processing methods of the present invention as described in further detail hereinafter. Placental tissue collected from the whole placenta was disaggregated by mechanical separation. Placental cells were collected and concentrated into a population of placental cells, which were then cultured as described hereinafter in further detail. As illustrated by the representative flow cytometry histograms in FIGS. 17a through 17e, FICOL indicates that CD117 is expressed in 0.5% of the 23a cells and CD44 is expressed in 25.8% of the 23a cells, which are 98.2% viable. At Passage 0, the cells of Cell Line 23a are shown to express CD117 at 12.9% and CD44 at 16.7% with 91.5% viability.

FIGS. 18a through 18o show flow cytometry data for Cell Line 23b, analyzed for cellular expression of CD44, CD45 and CD117 along with cell viability with 7AAD according to the methods of the present invention. Cell Line 23b was obtained by enzymatic digestion of a piece of placental tissue comprising maternal placental tissue, collecting and concentrating placental cells as a population of placental cell, and then culturing the placental cells according to the methods and processes of the present invention and as described hereinafter in further detail. The placental cells went through at least 39 passages in culture according to the methods of the present invention and as described in further detail hereinafter. Flow cytometry analysis was performed at multiple passages of the cell culture as shown in Table 8. The lowest calculated percentage of CD117 express occurred at FICOL and was 0.4% of the 23b cells with 91.5% viability. Flow Cytometry analysis of FICOL is shown in FIGS. 18a through 18e. The highest percentage of CD117 expression in the 23b cells occurred at Passage 2 and was 15.5%. Flow cytometry analysis of the cells of Cell Line 23a at Passage 2 is shown in FIGS. 18f through 18j. A high percentage of CD117 expression in the 23b cells also occurred at Passage 11. Flow cytometry analysis of the cells of Cell line 23a is shown in FIGS. 18k through 18o. Throughout cell culture, the cells maintained a high degree of viability as shown through 7AAD testing.

FIG. 19a through 19o show representative flow cytometry results for the cells of Cell Line PLE02, analyzed for cellular expression of CD44, CD45 and CD117 along with cell viability with 7AAD according to the methods of the present invention. The cells of Cell Line PLE02 were obtained by enzymatically digesting a piece of placental tissue comprising maternal placental tissue, collecting and concentrating placental cells as a population of placental cells, and cryopreserving the population of cells. The cryopreserved population of cells were thawed and concentrated with a density gradient centrifugation. The concentrated cells were then cultured through multiple passages that developed into Cell Line PLE02. FIGS. 19a through 19e show flow cytometry results of the analysis of cells at passage 5 of the cell culture of Cell Line PLE02, and FIGS. 19f through 19j show flow cytometry results of the analysis of cells at passage 7 of the cell culture of Cell Line PLE02. Cells of the cell culture were immunoselected for CD117 using the antibodies specific for CD117 using an MS Column (Miltenyi Biotec) according to the CD117 Cell Selection and CD117 Cell Separation methods of the present invention. The positive fraction of the immunoselected cells were analyzed with flow cytometry using antibodies specific for the cell surface markers CD44, CD45 and CD177. FIGS. 19k through 19o show flow cytometry results of the analysis of the positive fraction of the immunoselected cells. The cells were viable and expressed CD44 and CD117 and had low or no expression of CD45. Flow cytometry results for certain passages of the cell culture of Cell Line PLE02 are summarized in Table 11.

FIG. 20a through 20e show representative flow cytometry results for the cells of Cell Line PLE03, analyzed for cellular expression of CD44, CD45 and CD117 along with cell viability with 7AAD according to the methods of the present invention. The cells of Cell Line PLE03 were obtained by enzymatically digesting a piece of placental tissue comprising maternal placental tissue, collecting and concentrating placental cells as a population of placental cells, and cryopreserving the population of cells. The cryopreserved population of cells were thawed and concentrated with a density gradient centrifugation. The concentrated cells were then cultured through multiple passages and developed into Cell Line PLE03. FIGS. 20a through 20e show flow cytometry results of the analysis of cells at passage 7 of the cell culture of Cell Line PLE03. The cells were viable and expressed CD44 and CD117 and had low or no expression of CD45. Flow cytometry results for certain passages of the cell culture of Cell Line PLE03 are summarized in Table 12.

FIGS. 21a through 21e show representative flow cytometry results for the cells of Cell Line PLE04, analyzed for cellular expression of CD44, CD45 and CD117 along with cell viability with 7AAD according to the methods of the present invention. The cells of Cell Line PLE04 were obtained by enzymatically digesting a piece of placental tissue comprising maternal placental tissue, collecting and concentrating placental cells as a population of placental cells, and cryopreserving the population of cells. The cryopreserved population of cells were thawed and concentrated with a density gradient centrifugation. The concentrated cells were then cultured through multiple passages that developed into Cell Line PLE04. FIGS. 21a through 21e show flow cytometry results of the analysis of cells at passage 9 of the cell culture of Cell Line PLE04. The cells were viable and expressed CD44 and CD117 and had low or no expression of CD45. Flow cytometry results for certain passages of the cell culture of Cell Line PLE04 are summarized in Table 13.

FIG. 22a through 22t show representative flow cytometry results for the cells of Cell Line PLE05, analyzed for cellular expression of CD44, CD45 and CD117 along with cell viability with 7AAD according to the methods of the present invention. The cells of Cell Line PLE05 were obtained by enzymatically digesting a piece of placental tissue comprising maternal placental tissue, collecting and concentrating placental cells as a population of placental cells, and cryopreserving the population of cells. The cryopreserved population of cells were thawed and concentrated with a density gradient centrifugation. The concentrated cells were then cultured through multiple passages that developed into Cell Line PLE05. The cultures cells were immunoselected for CD117 at several points in the culture. FIGS. 22a through 22e show flow cytometry results of the analysis of cells at passage 9 of the cell culture of Cell Line PLE05 after the cells were immunoselected for CD117 using antibodies for CD117 using an MS Column (Miltenyi Biotec) according to the CD 117 Cell Selection and CD117 Cell Separation methods of the present invention. FIGS. 22f through 22j show flow cytometry results for the analysis of cells obtained in a positive fraction at passage 1 after immunoselection for CD117. FIGS. 22k through 22o show flow cytometry results for the analysis of cells obtained in a positive fraction at passage 5 prior to double immunoselection. FIGS. 22p through 22t show flow cytometry results for the analysis of cells obtained in a positive fraction at passage 0 with double immunoselection. The cells were viable and expressed CD44 and CD117 and had low or no expression of CD45. Flow cytometry results for certain passages of the cell culture of Cell Line PLE05 are summarized in Table 14.

FIG. 23a through 23ii show representative flow cytometry results for the cells of Cell Line PLE06, analyzed for cellular expression of CD44, CD45 and CD117 along with cell viability with 7AAD according to the methods of the present invention. The cells of Cell Line PLE06 were obtained by enzymatically digesting a piece of placental tissue comprising maternal placental tissue, collecting and concentrating placental cells as a population of placental cells, and cryopreserving the population of cells. The cryopreserved population of cells were thawed and concentrated with a density gradient centrifugation. The concentrated cells were then cultured through multiple passages that developed into Cell Line PLE06 and immunoselected for CD117. FIGS. 23a through 23e show flow cytometry results at passage 6 of culture. FIGS. 23f through 23j show flow cytometry results at passage 8 of culture. FIGS. 23k through 23o show flow cytometry results of a positive fraction at passage 2 of culture after immunoselection. FIGS. 23p through 23t show flow cytometry results of a positive fraction at passage 5 of culture after immunoselection. FIGS. 23u through 23y show flow cytometry results of a positive fraction at passage 6 of culture after immunoselection. FIGS. 23z through 23dd show flow cytometry results of a positive fraction at passage 8 of culture after immunoselection. FIGS. 23ee through 23ii show flow cytometry results of a positive fraction at passage 9 of culture after immunoselection. The cells were viable and expressed CD44 and CD117 and had low or no expression of CD45. Flow cytometry results for certain passages of the cell culture of Cell Line PLE06 are summarized in Table 15.

FIGS. 24a through 24d illustrate genotyping of cells of Cell Line PLE02 performed by Human Identification-Multiplex Short Tandem Repeat (STR) Analysis. The STR Analysis involved the performance of PCR analysis on 15 different STR loci plus amelogenin on the X and Y chromosomes. The 15 STR loci analyzed were D8S1179, D21S11, D7S820, CSF1P0, D3S1358, TH01, D13S317, D16S539, D2S1338, D19S433, vWA, TPOX, D18S51, D5S818, and FGA. The amplified product was electrophoresed on ABI 3100 Genetic Analyzer and analyzed using the GeneMapper ID software. Four separate fluorescent dye labels were used to label the samples. The dyes are coupled to PCR primers. Each of these fluorescent dyes emitted its maximum fluorescence at a different wavelength, that was detected by the Genetic Analyzer. The analysis was performed by visual inspection of all 15 loci plus amelogenin (X and Y marker). The results show that the individual associated with the cells of the PLE02 Cell Line is 100% female and 0% male based on the amelogenin locus analysis, which indicated that only the X marker is present. In addition, the specimen is of single individual origin. This was established by looking at the STR data for each locus. Each locus (designated by the gray bar at the top of each graph) for a normal individual should have one or two STR alleles. (Individual is homozygous if only one marker is present). PLE-02 has alleles 13 and 14 present at D8S1179 locus.

FIG. 25a through 23d illustrate genotyping of cells of Cell Line PLE03 performed by Human Identification-Multiplex Short Tandem Repeat (STR) Analysis. The STR Analysis involved the performance of PCR analysis on 15 different STR loci plus amelogenin on the X and Y chromosomes as previously discussed in FIGS. 24a through 24d. The individual associated with the cells of the PLE03 Cell Line is 100% female and 0% male based on amelogenin locus analysis, which indicated that only the X marker is present. In addition, the specimen is of single individual origin. This was established by looking at the STR data for each locus. Each locus (designated by the gray bar at the top of each graph) for a normal individual should have one or two STR alleles. (Individual is homozygous if only one marker is present). PLE-03 has alleles 13 and 14 present at D8S1179 locus.

FIGS. 26a through 26d illustrate genotyping of cells of Cell Line PLE04 performed by Human Identification-Multiplex Short Tandem Repeat (STR) Analysis. The STR Analysis involved the performance of PCR analysis on 15 different STR loci plus amelogenin on the X and Y chromosomes as previously discussed in FIGS. 24a through 24d. The individual associated with the cells of the PLE04 Cell Line is 100% female and 0% male based on amelogenin locus analysis, which indicated that only the X marker is present. In addition, the specimen is a mixed chimera. This was established by looking at the STR data for each locus. Each locus (designated by the gray bar at the top of each graph) for a normal individual should have one or two STR alleles. (Individual is homozygous if only one marker is present). PLE-04 has alleles 13 and 14 present at D8S1179 locus.

FIGS. 27a through 27d illustrate genotyping of cells of Cell Line PLE05 performed by Human Identification-Multiplex Short Tandem Repeat (STR) Analysis. The STR Analysis involved the performance of PCR analysis on 15 different STR loci plus amelogenin on the X and Y chromosomes as previously discussed in FIGS. 24a through 24d. The individual associated with the cells of the PLE05 Cell Line is 100% female and 0% male based on amelogenin locus analysis, which indicates that only the X marker is present. In addition, the specimen is of single individual origin. This was established by looking at the STR data for each locus. Each locus (designated by the gray bar at the top of each graph) for a normal individual should have one or two STR alleles. (Individual is homozygous if only one marker is present). PLE-05 has alleles 13 and 14 present at D8S1179 locus.

FIGS. 28a through 28d illustrate genotyping of cells of Cell Line PLE06 performed by Human Identification-Multiplex Short Tandem Repeat (STR) Analysis. The STR Analysis involved the performance of PCR analysis on 15 different STR loci plus amelogenin on the X and Y chromosomes as previously discussed in FIGS. 24a through 24d. The individual associated with the cells of the PLE03 Cell Line is 100% female and 0% male based on amelogenin locus analysis, which indicates that only the X marker is present. In addition, the specimen is of single individual origin. This was established by looking at the STR data for each locus. Each locus (designated by the gray bar at the top of each graph) for a normal individual should have one or two STR alleles. (Individual is homozygous if only one marker is present). PLE-06 has alleles 13 and 14 present at D8S1179 locus.

FIG. 29 shows immunostaining of cells selected from Cell Lines PLE02, PLE03, PLE04, PLE05 and PLE06 for Oct4 using labeled anti-Oct4 antibodies. Cell Lines PLE02, PLE03, PLE04, PLE05 and PLE06 showed no Oct4 expression.

FIGS. 30a through 30c represent testing of human amniotic cell line A1 and cells selected from Cell Line PLE02, PLE03, PLE04, PLE05 and PLE06 for their ability to differentiate into adipogenic, osteogenic and nervous system cells.

FIG. 30a represents testing of human amniotic cell line A1 and cells selected from Cell Line PLE02, PLE03, PLE04, PLE05 and PLE06 for their ability to differentiate into nervous system cells. Cells from all of the Cell Lines PLE02 through PLE06 were grown in media known to induce differentiation into nervous system cells. Differentiation can be illustrated by induction of expression of nestin, a cytoskeletal protein present in neural stem cells. The Cell Lines were tested for expression of nestin by immunostaining with anti-nestin antibodies. Positive staining was shown through immunofluorescence after growth of the cells in the induction media for all Cell Lines PLE02 through PLE06.

FIG. 30b represents testing of human amniotic cell line A1 and cells selected from Cell Line PLE02, PLE03, PLE04, PLE05 and PLE06 for their ability to differentiate into bone cells. Cells from all of the Cell Lines were grown in media known to induce differentiation into cells of the osteogenic lineage. The cells were tested for osteogenic differentiation by Alizarin-red staining used for detecting induction of calcification deposits in the cells of the Cell Lines. Calcification was shown for all Cell Lines except PLE02.

FIG. 30c represents testing of human amniotic cell line A1 and cells selected from Cell Line PLE02, PLE03, PLE04, PLE05 and PLE06 for their ability to differentiate into fat cells. Cells from all of the Cell Lines were grown in media known to induce differentiation of cells into the adipogenic lineage. The cells were tested for adipogenic differentiation by Oil-red-O staining for showing intracellular lipid accumulation. Intracellular lipid accumulation was shown for all Cell Lines.

FIG. 31 shows qualitative PCR (Q-PCR) analysis of in vitro differentiation of human amniotic cell line A1 and cell lines PLE02, PLE03, PLE04, PLE05 and PLE06 along the osteogenic cell lineage. RNA was obtained from the cells of the Cell Lines and analyzed by Q-PCR for expression of Runx2 (Cbfa1), a characteristic transcription factor for cells along the osteoblastic lineage, and alkaline phosphatase, an enzyme known to associate with mineralization. All Cell Lines showed expression of Runx2 expression. Cell Lines PLE03, PLE04 and PLE05 showed an increase in alkaline phosphatase expression.

FIG. 32 shows qualitative PCR (Q-PCR) analysis of in vitro differentiation of human amniotic cell line A1 and cell lines PLE02, PLE03, PLE04, PLE05 and PLE06 along the adipogenic lineage. RNA was obtained from the cells of the Cell Lines and analyzed by Q-PCR for expression of peroxisome proliferators-activated receptor gamma (PPAR gamma), a free fatty acid receptor, and lipoprotein lipase (LPL), an enzyme capable of hydrolyzing lipids into lipoproteins into three fatty acids and a glycerol molecule. The cells of the Cell Lines PLE02 through PLE06 showed an increase is LPL expression during conditions to induce differentiation of cells into the adipogenic lineage. PPAR gamma expression did not increase in any of the cells of the Cell Lines during conditions to induce differentiation of the cells into the adipogenic lineage.

### DESCRIPTION OF THE PREFERRED EMBODIMENTS

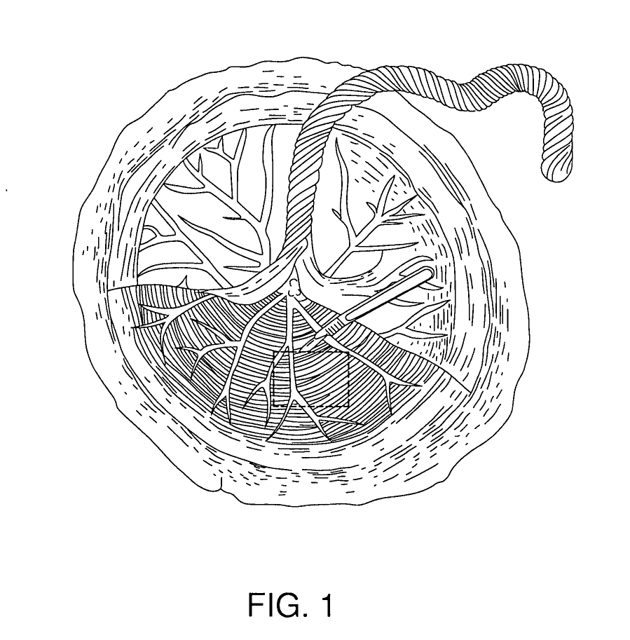

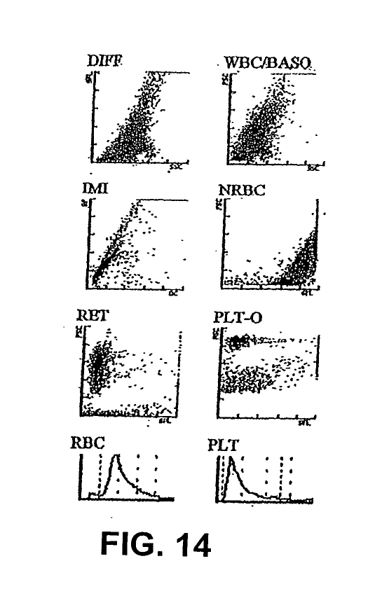

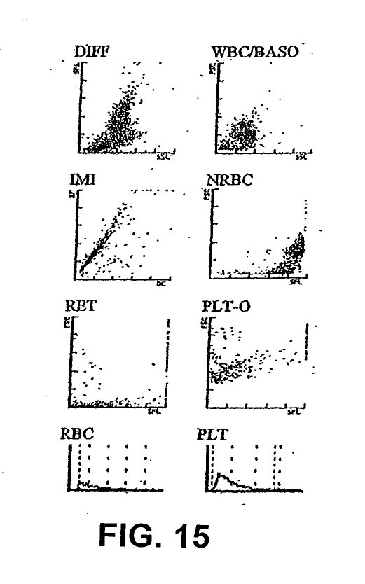

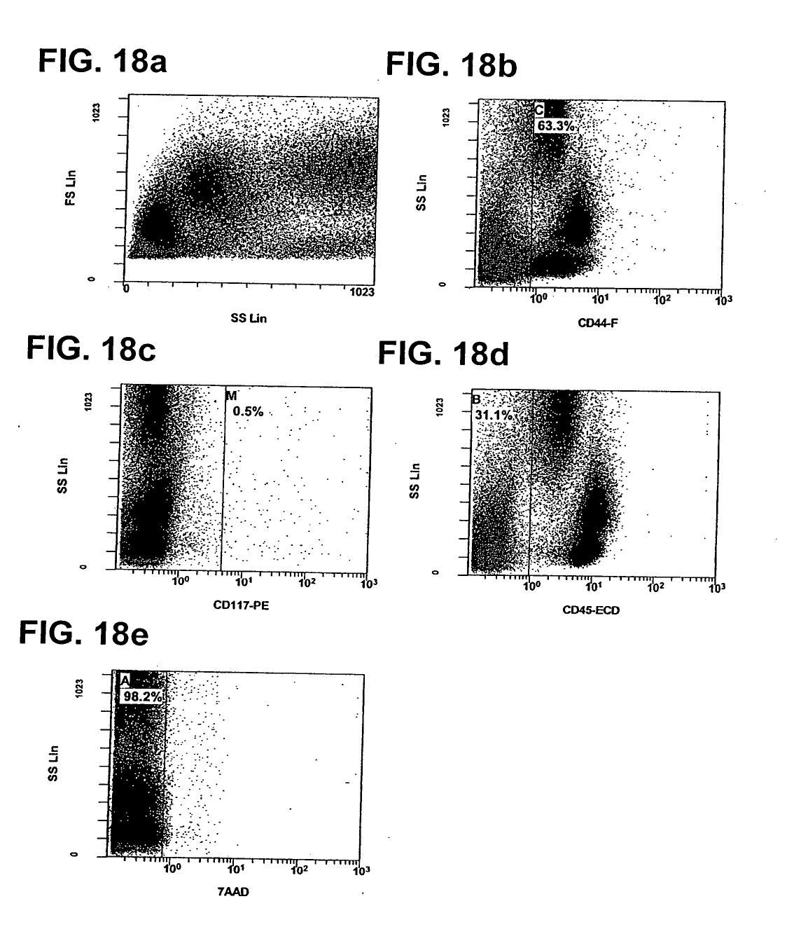

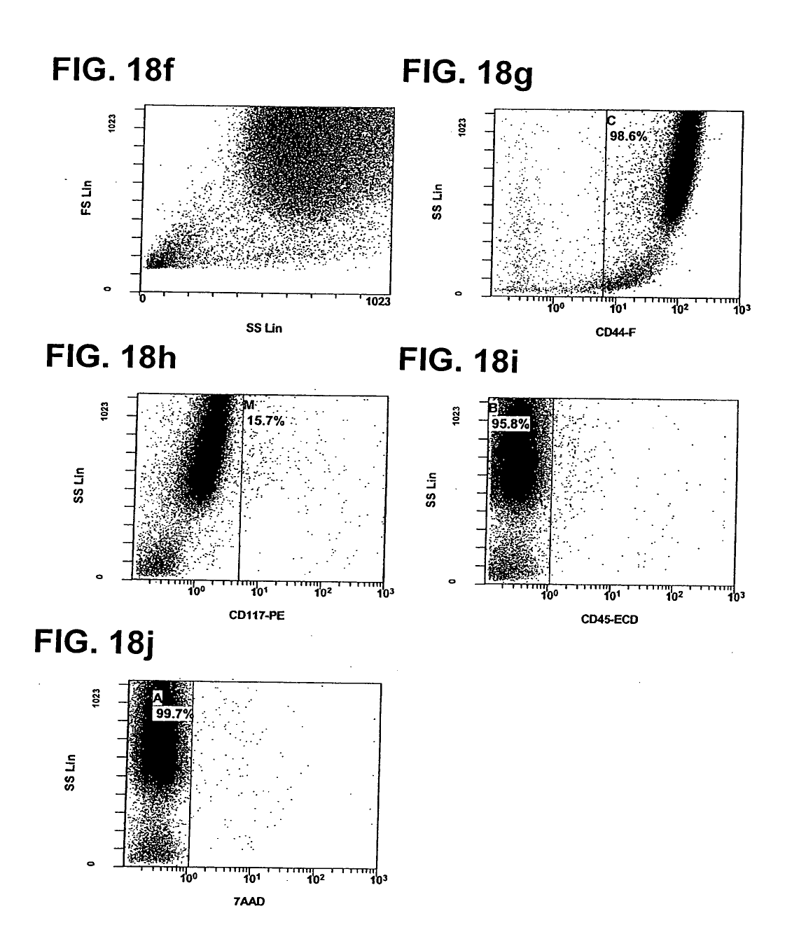

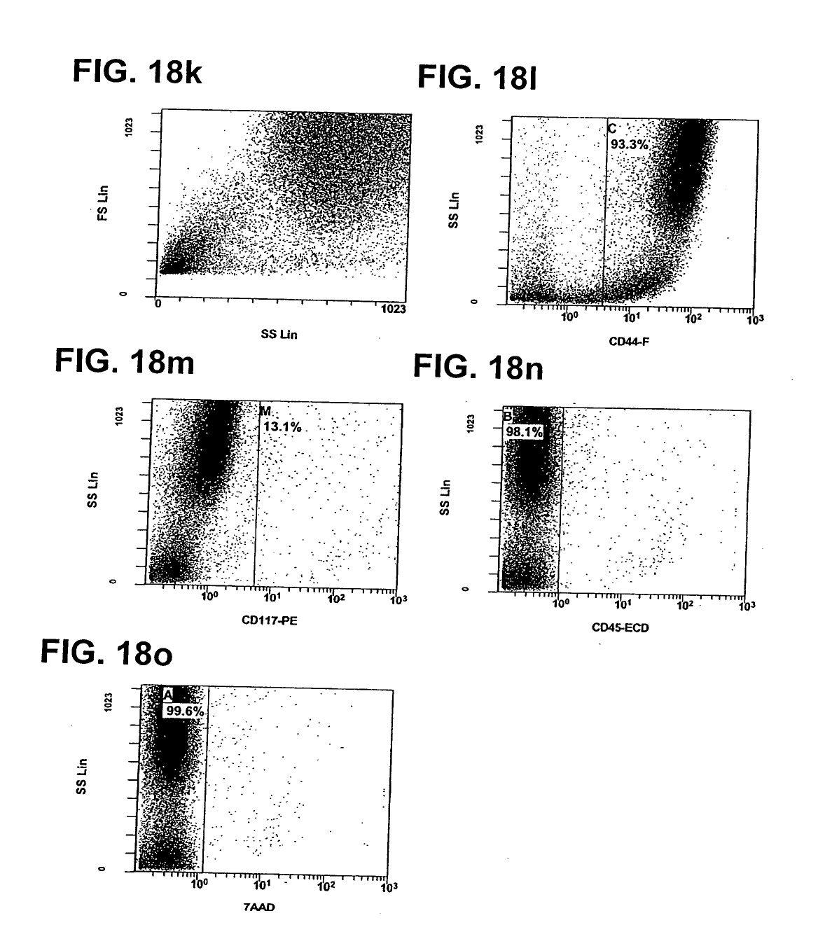

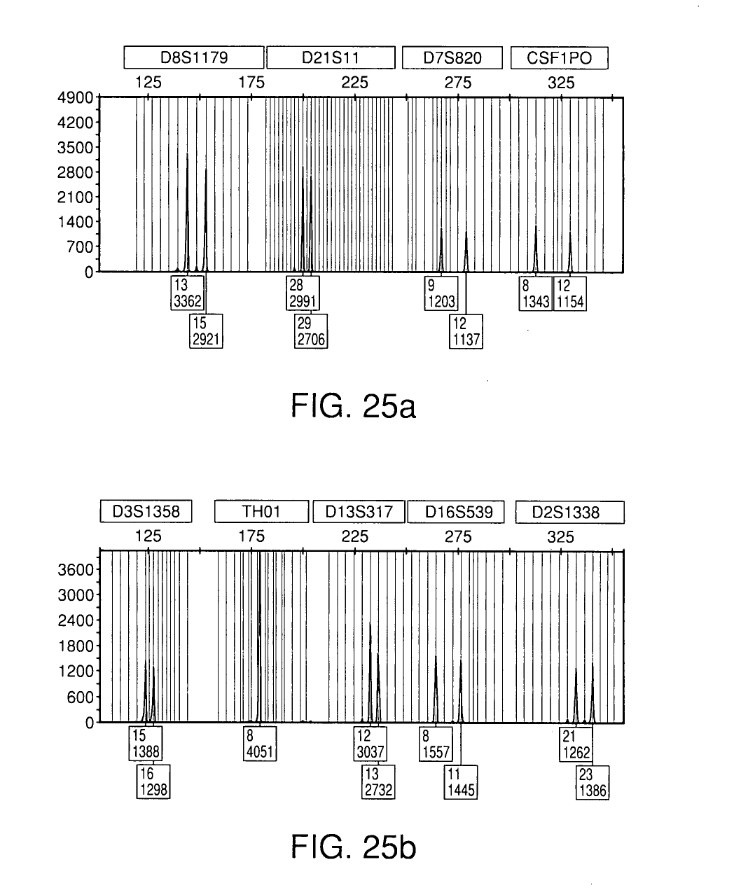

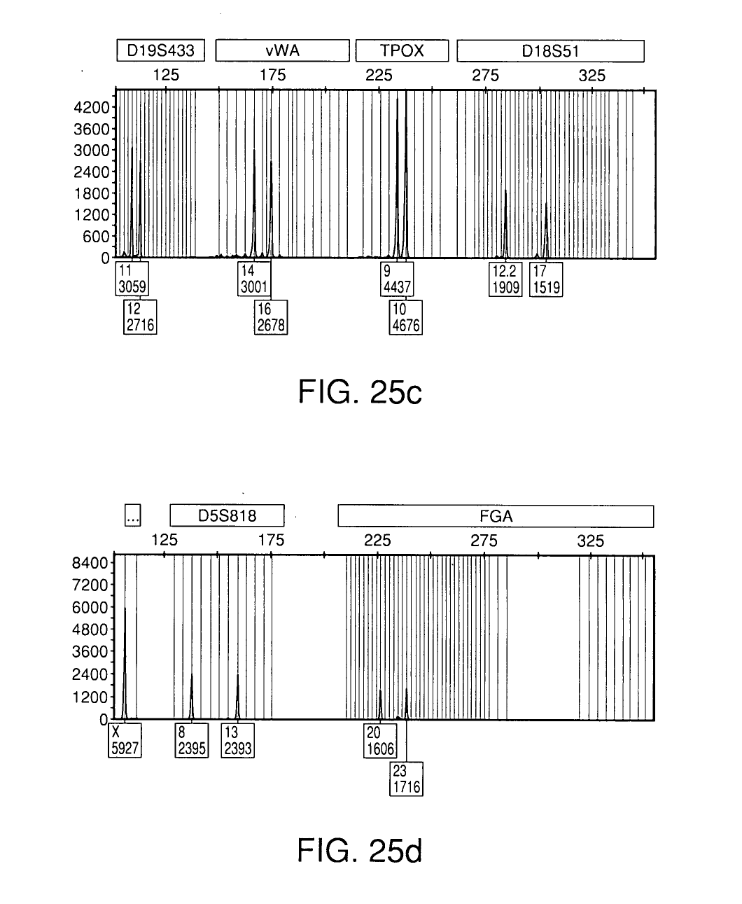

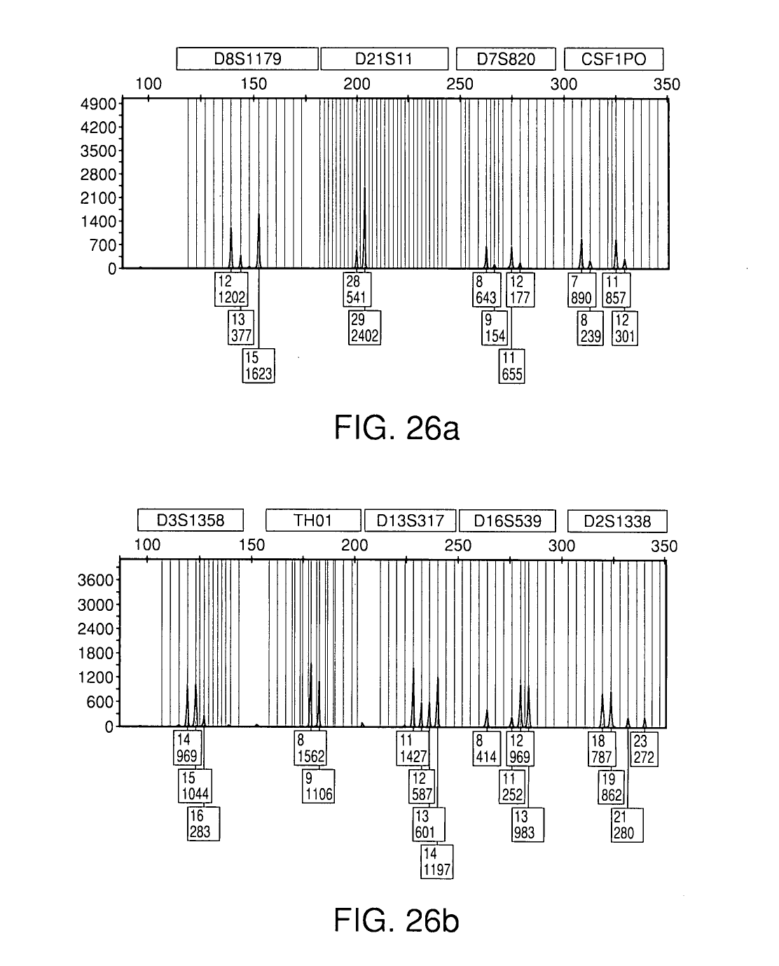

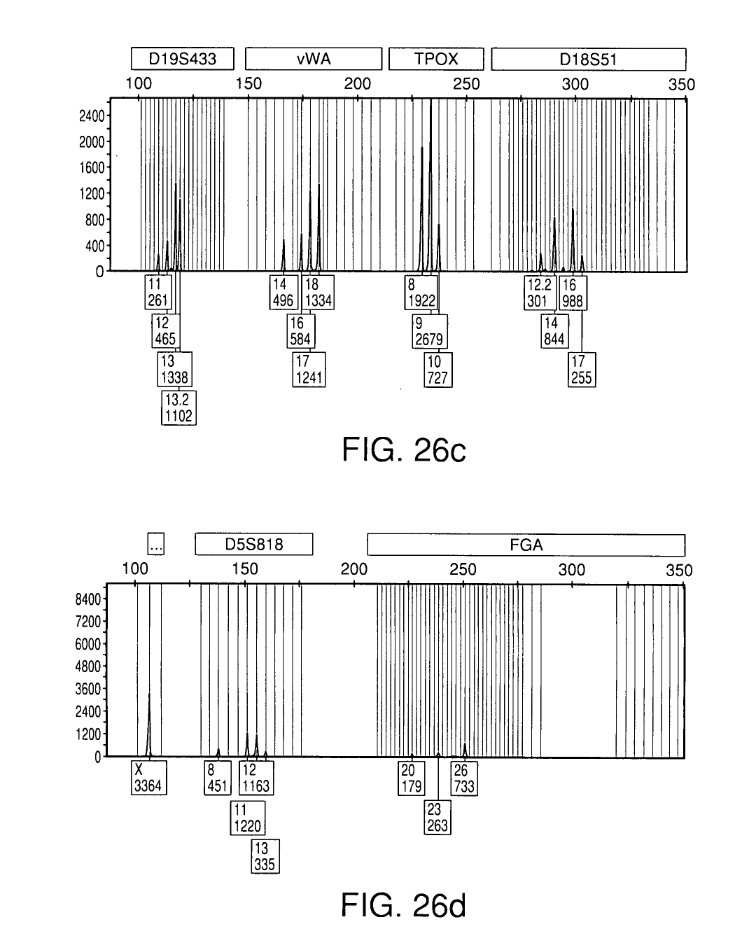

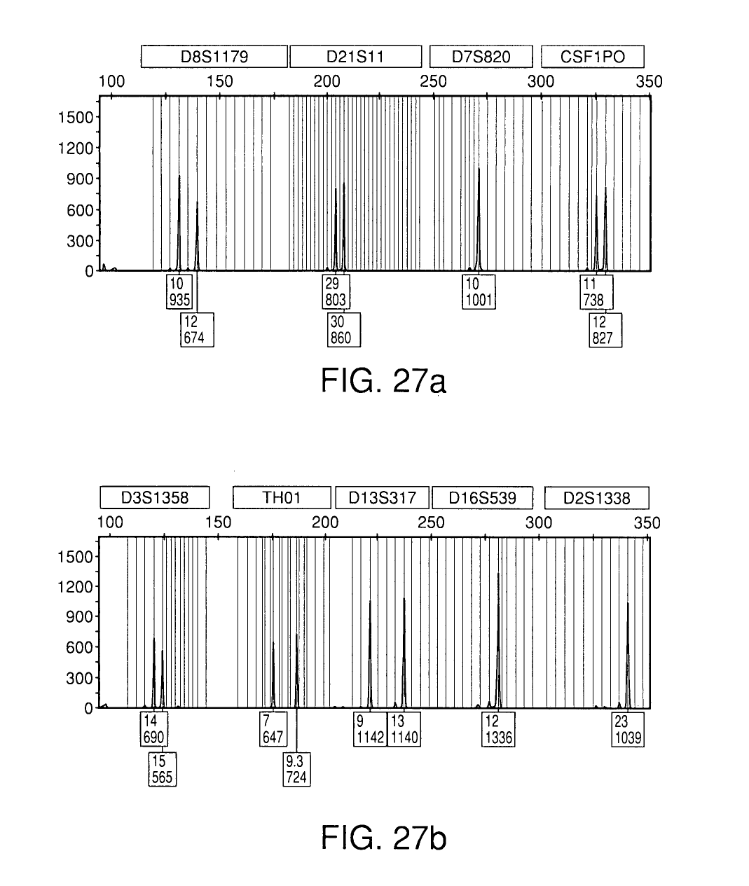

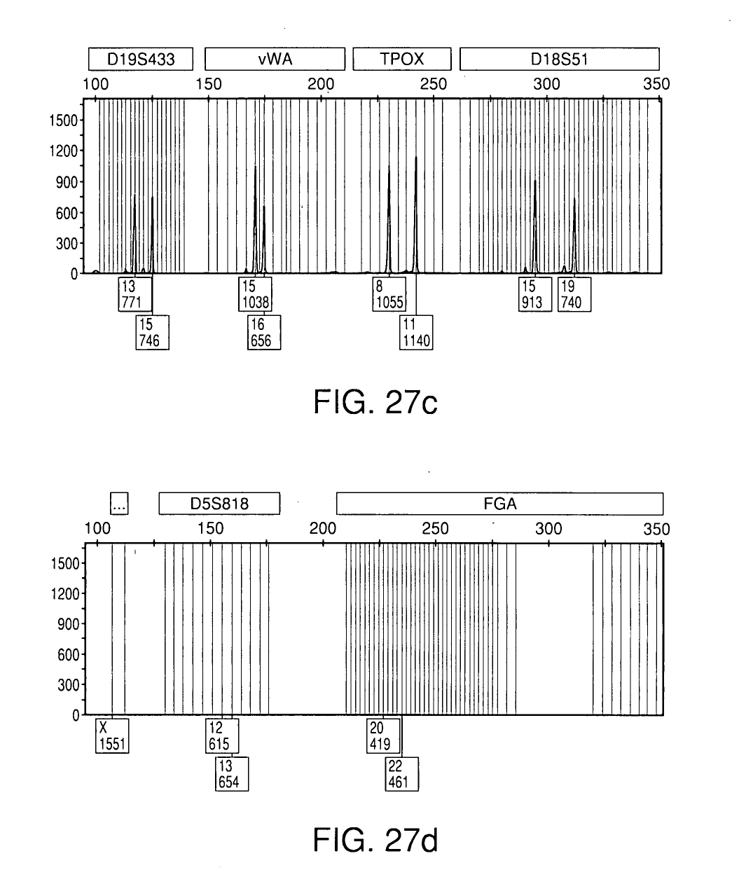

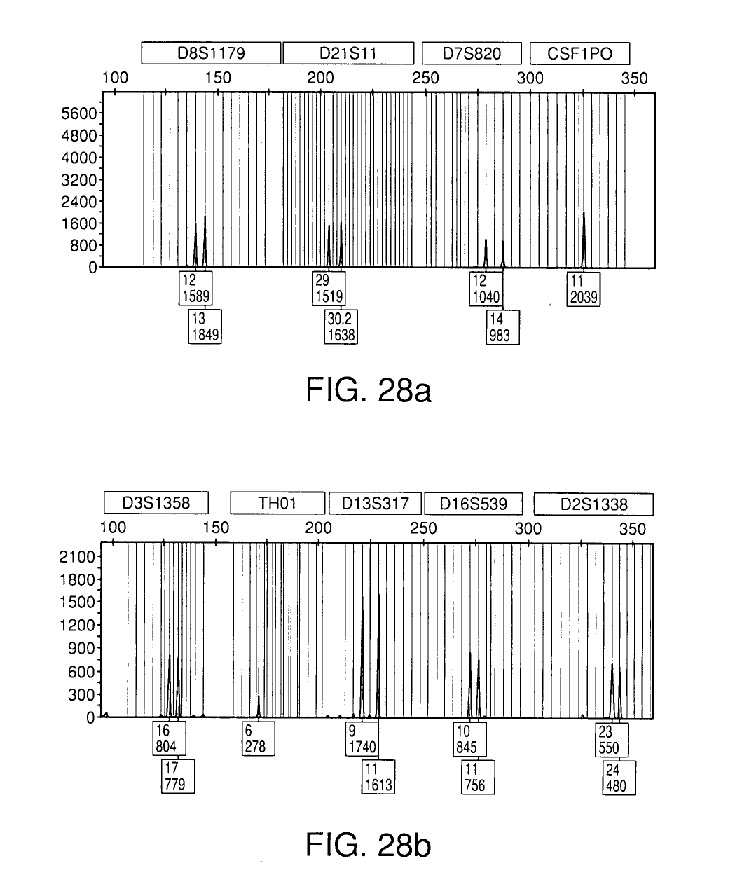

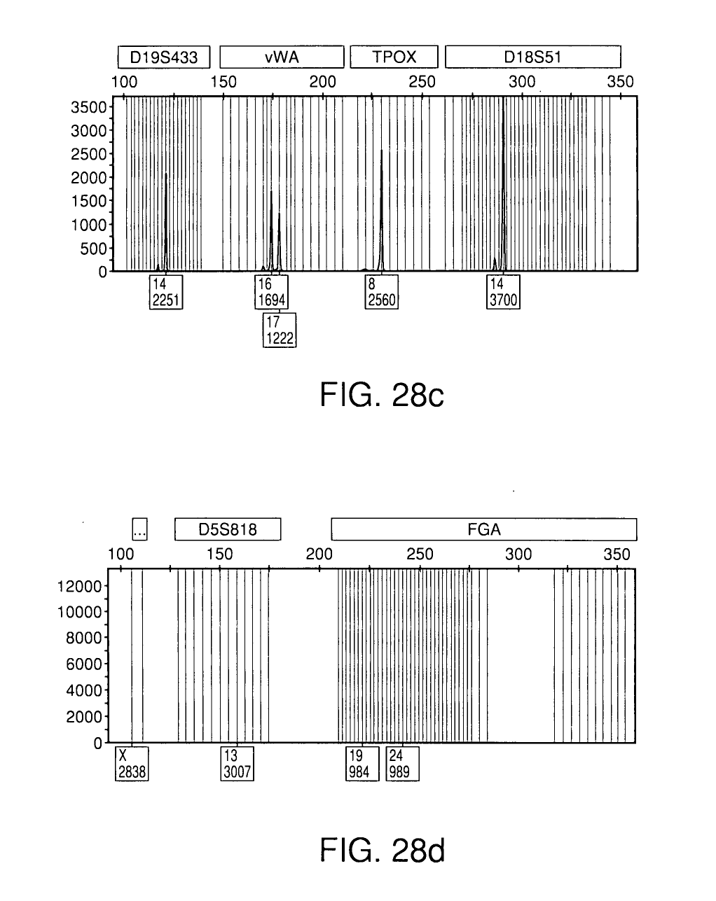

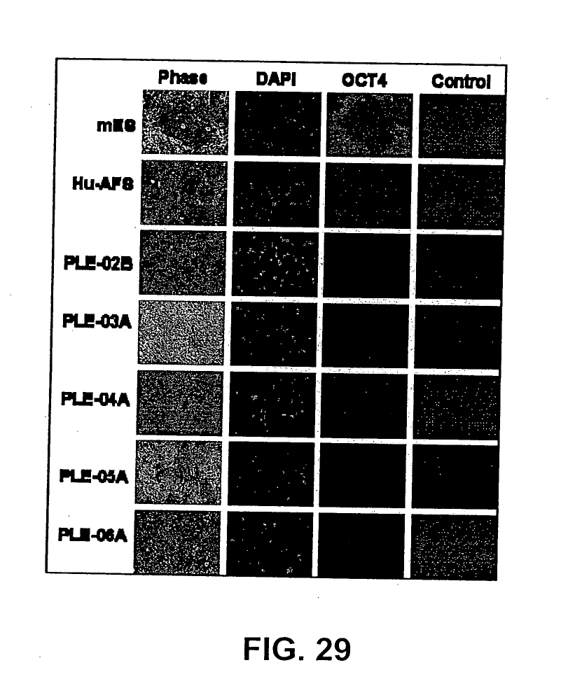

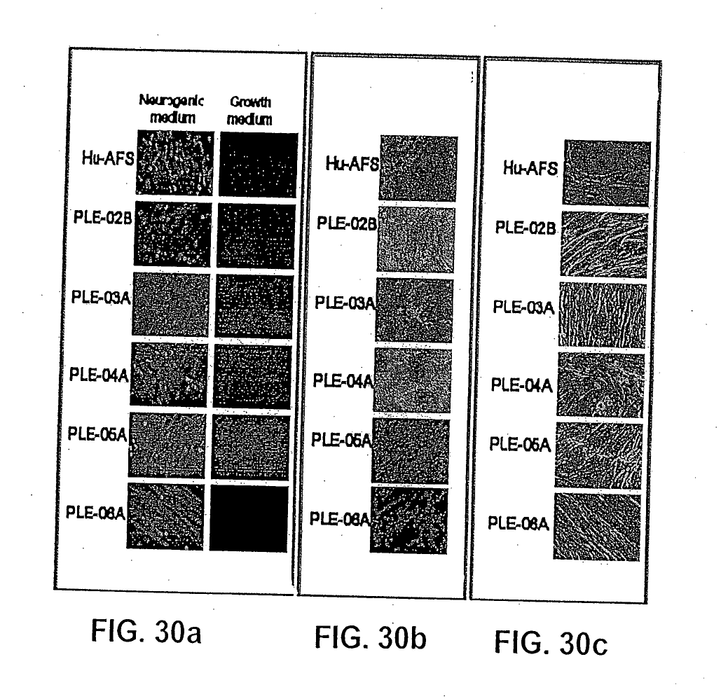

In reference to FIGS. 1 through 32, the present invention provides methods, processes, and systems for the procurement and processing of placental tissue obtained from a whole human placenta and isolating, collecting and preserving a population of placental cells comprising viable, multipotent maternal placental cells obtained from the placental tissue. It is recognized that certain maternal placental stem cells collected in accordance with the present invention are at least multipotent in nature due to their ability to differentiate into certain cell lineages as demonstrated in FIGS. 30a through 32. The term “multipotent” is used in reference to the ability of a cell to differentiate into more than one cell type of the body under suitable conditions for differentiation induction.

In reference to the present invention, the word “cell” generally, and the term “placental cell” specifically, is used to refer to any cell or cells obtained and selected by the methods, processes and systems of the present invention. Cells and placental cells include maternal placental stem cells. The phrases “maternal placental stem cell” or “MPSC” are used to refer to a cell or placental cell obtained from maternal tissue of the placenta. The maternal placental stem cell expresses at least one of the cell surface markers selected from the group consisting of CD29, CD44, CD73, CD105, CD117, SSEA-3 and SSEA-4 and has low or no expression of at least one of the cell surface markers CD34, CD45, CD133, TRA-1-60 and TRA-1-81.

Maternal placental stem cells express the antigenic factor CD117 that is also known as a c-kit receptor, Steel factor receptor, and stem cell factor receptor. The gene for c-kit encodes a tyrosine kinase growth factor receptor for Stem Cell Factor (SCF), which is also known as mast cell growth factor and is essential for hematopoiesis, melanogenesis, and fertility. It is recognized that CD117 is expressed in hematopoietic stem cells, mast cells, germ cells, melanocytes, certain basal epithelial cells, luminal epithelium of the breast, and the interstitial cells of Cajal of the gastrointestinal tract. CD117 imparts a critical role in germ cell establishment, maintenance, and function. Research indicates that in the embryonic gonad, CD117 and its corresponding ligand SCF, are essential for the primordial germ cell survival and proliferation. Additionally, research indicates that CD117 and its corresponding ligand SCF are essential for the gamete production in response to gonadotropic hormones. In other words, CD117 in combination with the ligand SCF are necessary for the survival and proliferation of germ cells of the testis, the spermatogonia, and for the growth and maturation of oocytes. Research also indicates that CD117 is a potent growth factor for primitive hematopoietic cell proliferation in vitro. Accordingly, the placental cells which include maternal placental stem cells of the present invention are a viable source of cells due to their inherent characteristics.

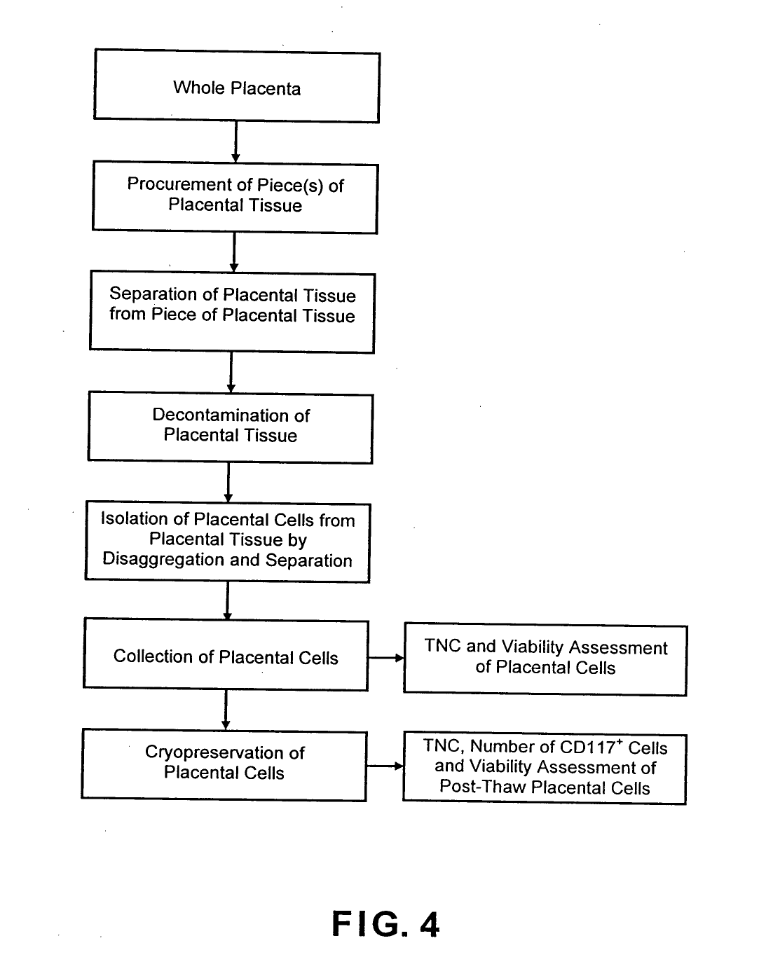

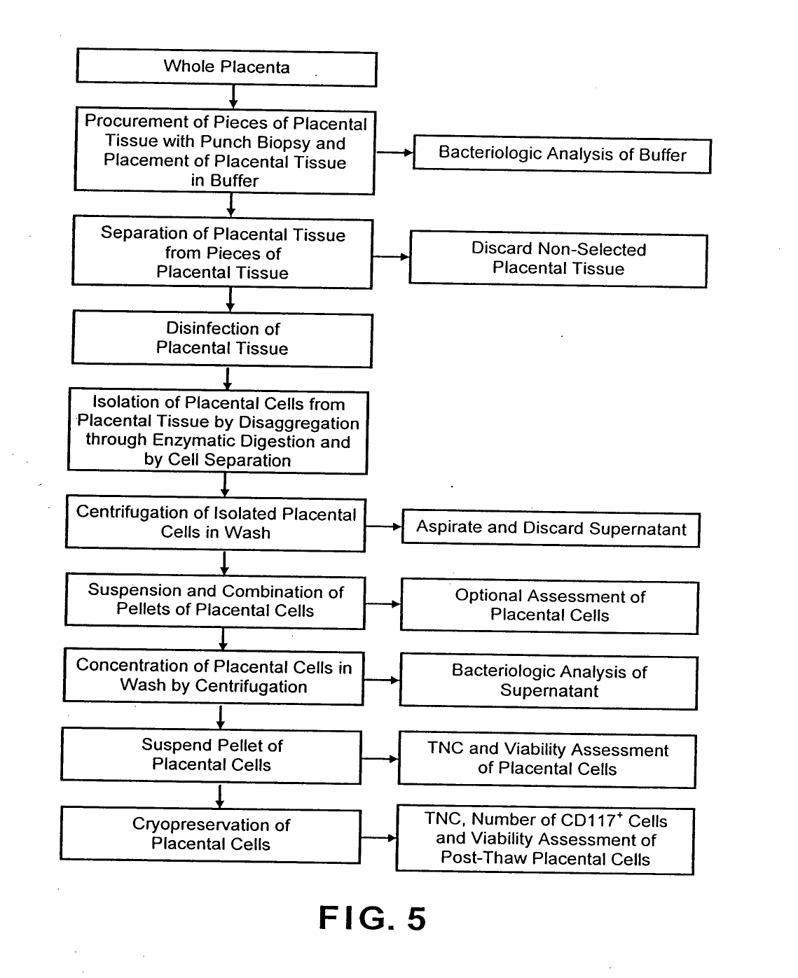

Generally, and as illustrated in FIG. 4, the overall methods, processes and systems of the present invention involve procurement of a piece of placental tissue from a whole placenta, optional separation of placental tissue from the piece of placental tissue; decontamination of the piece of placental tissue; isolation of placental cells from the piece of placental tissue by disaggregation and separation; collection and optionally cryopreservation of a population of cells comprising maternal placental cells. Pre-processing samples and post-processing samples of collected placental cells are analyzed with flow cytometry for specific cell surface markers and other proteins as described herein. Bacteriological analysis of the environment of the placental tissue and placental cells is performed to identify any microbial contamination of the piece of placental tissue and placental cells collected by the methods and processes of the present invention. The overall methods and processes of the present invention are further detailed in various embodiments as illustrated in FIGS. 5 through 12.

The embodiment of the present invention illustrated in FIG. 5 generally involves obtaining placental tissue comprising at least maternal tissue by punch biopsy and disaggregating the placental tissue by enzymatic digestion of the placental tissue to release placental cells. After the enzymatic reaction is inhibited with an inhibitor, the placental cells are isolated from the placental tissue by centrifugation in a wash. The placental cells comprising maternal placental stem cells are collected and concentrated through several centrifugation steps and optionally cryopreserved in the vapor of liquid Nitrogen. Such cryopreservation may occur at or below a temperature of about −135° C.

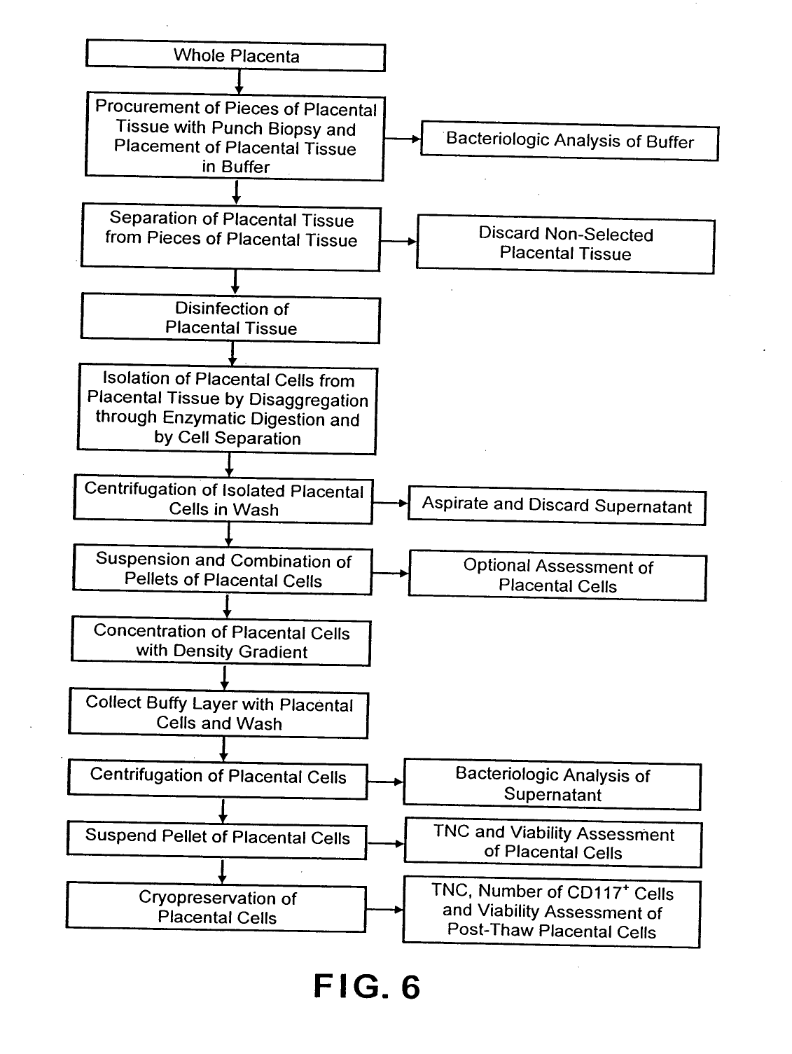

Another embodiment of the present invention illustrated in FIG. 6 generally involves obtaining placental tissue comprising at least maternal tissue by punch biopsy and disaggregating the placental tissue by enzymatic digestion of the placental tissue to release placental cells comprising maternal placental stem cells. After the enzymatic reaction is inhibited with an inhibitor, the placental cells comprising maternal placental stem cells are isolated from the placental tissue by centrifugation in a wash. The placental cells comprising maternal placental stem cells are collected with a density gradient in a buffy coat layer that is collected and concentrated through several centrifugation steps and optionally cryopreserved in the vapor of liquid Nitrogen. Such cryopreservation may occur at or below a temperature of about −135° C.

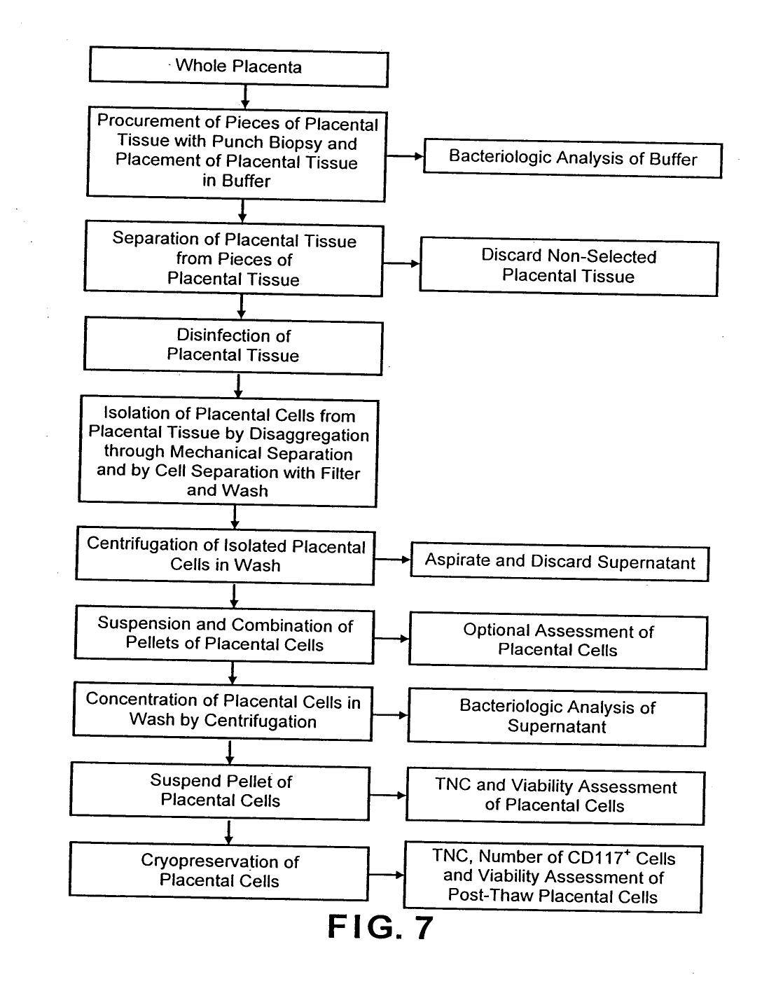

An additional embodiment of the present invention illustrated in FIG. 7 generally involves obtaining placental tissue comprising at least maternal tissue by punch biopsy and disaggregating the placental tissue by mechanical separation of the placental tissue to release placental cells comprising maternal placental stem cells. The placental cells comprising maternal placental stem cells are isolated from the placental tissue through cell separation with a filter and wash. The placental cells comprising maternal placental stems cells are collected and concentrated through several centrifugation steps and optionally cryopreserved in the vapor of liquid Nitrogen. Such cryopreservation may occur at or below a temperature of about −135° C.

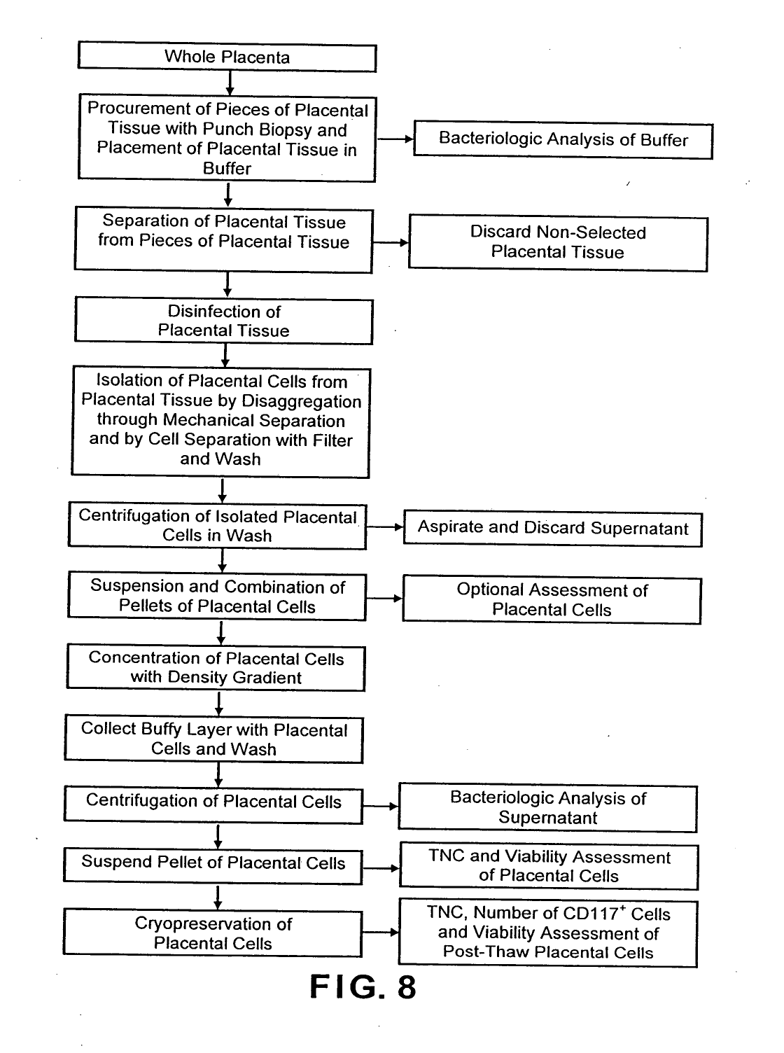

A further embodiment of the present invention illustrated in FIG. 8 generally involves obtaining placental tissue by punch biopsy and disaggregating the placental tissue by mechanical separation of the placental tissue to release placental cells comprising maternal placental stem cells. The placental cells comprising maternal placental stem cells are isolated from the placental tissue through cell separation with a filter and wash. The placental cells comprising maternal placental stem cells are collected with a density gradient in a buffy coat layer that is collected and concentrated through centrifugation steps and optionally cryopreserved in the vapor of liquid Nitrogen. Such cryopreservation may occur at or below a temperature of about −135° C.

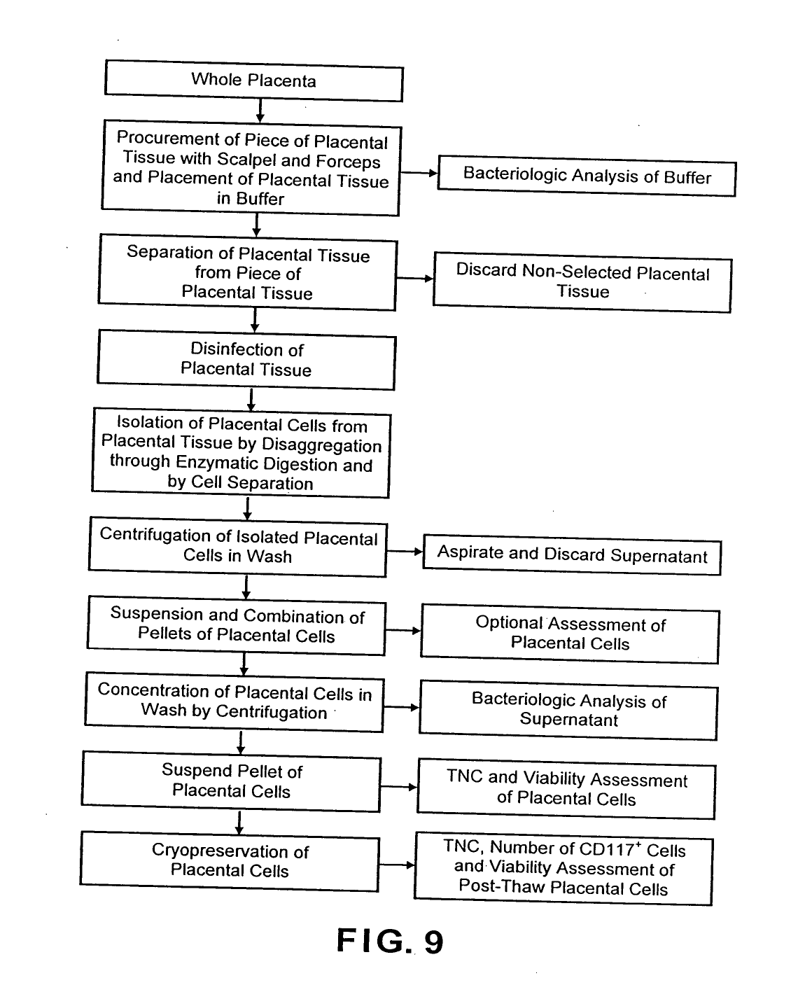

Yet another embodiment of the present invention illustrated in FIG. 9 generally involves obtaining placental tissue with scalpel and forceps and disaggregating placental tissue by enzymatic digestion of the placental tissue to release placental cells comprising maternal placental stem cells. After the enzymatic reaction is inhibited, the placental cells comprising maternal placental stem cells are isolated from the placental tissue by centrifugation in a wash. The placental cells comprising maternal placental stem cells are collected and optionally concentrated through centrifugation steps and optionally cryopreserved in the vapor of liquid Nitrogen. Such cryopreservation may occur at or below a temperature of about −135° C.

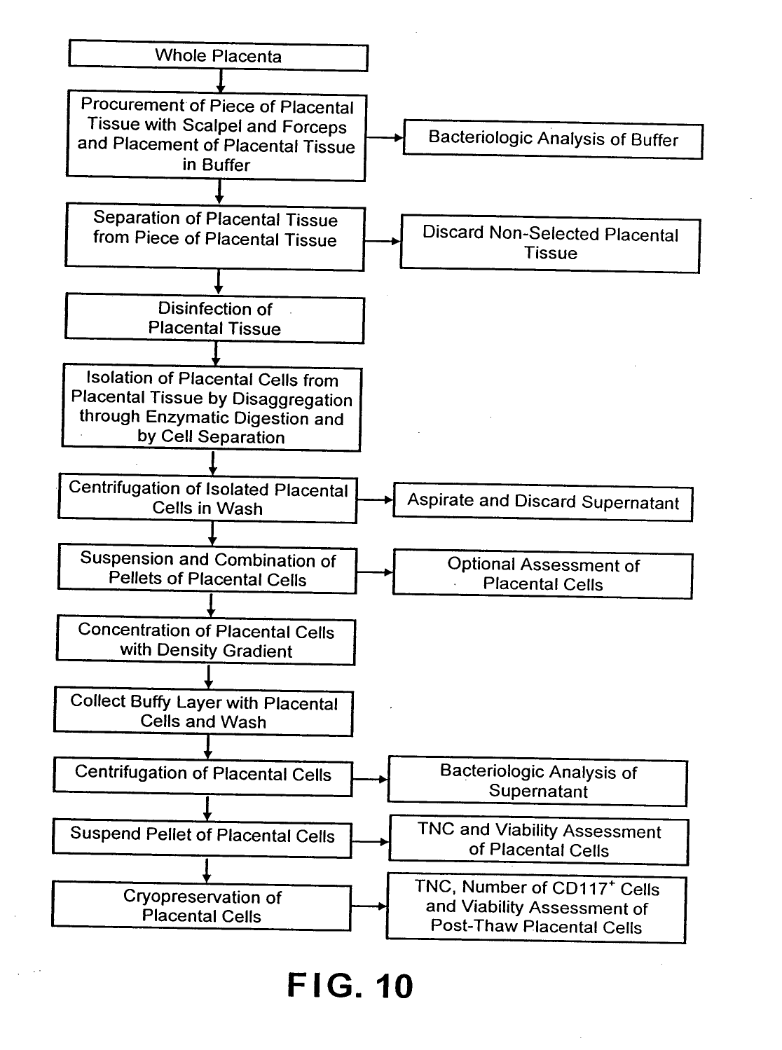

Yet an additional embodiment of the present invention illustrated in FIG. 10 generally involves obtaining placental tissue with scalpel and forceps and disaggregating the placental tissue by enzymatic digestion of the placental tissue to release placental cells comprising maternal placental stem cells. After the enzymatic reaction is inhibited, the placental cells comprising maternal placental stem cells are isolated from the placental tissue by centrifugation in a wash. The placental cells comprising maternal placental stem cells are collected with a density gradient in a buffy coat layer that is collected and concentrated through centrifugation steps and optionally cryopreserved in the vapor of liquid Nitrogen. Such cryopreservation may occur at or below a temperature of about −135° C.

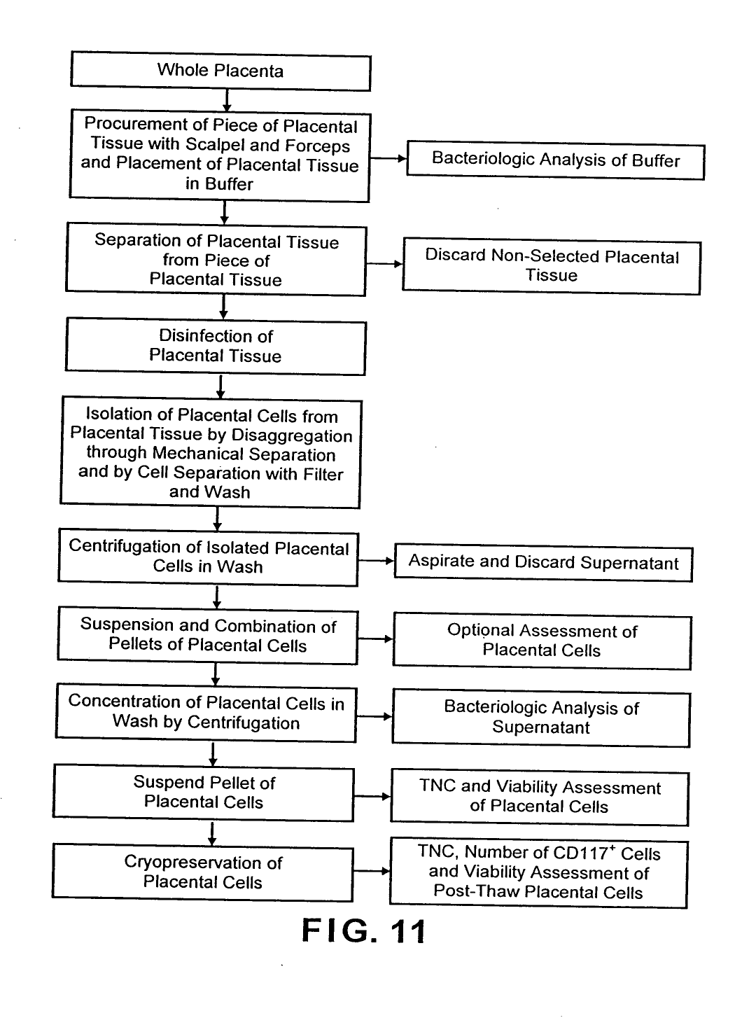

Yet a further embodiment of the present invention illustrated in FIG. 11 generally involves obtaining placental tissue with scalpel and forceps and disaggregating placental tissue by mechanical separation of the placental tissue to release placental cells comprising maternal placental stem cells. The placental cells comprising maternal placental stem cells are isolated from the placental tissue through cell separation with a filter and wash, and the placental cells comprising maternal placental stem cells are collected and concentrated through centrifugation steps and optionally cryopreserved in the vapor of liquid Nitrogen. Such cryopreservation may occur at or below a temperature of about −135° C.

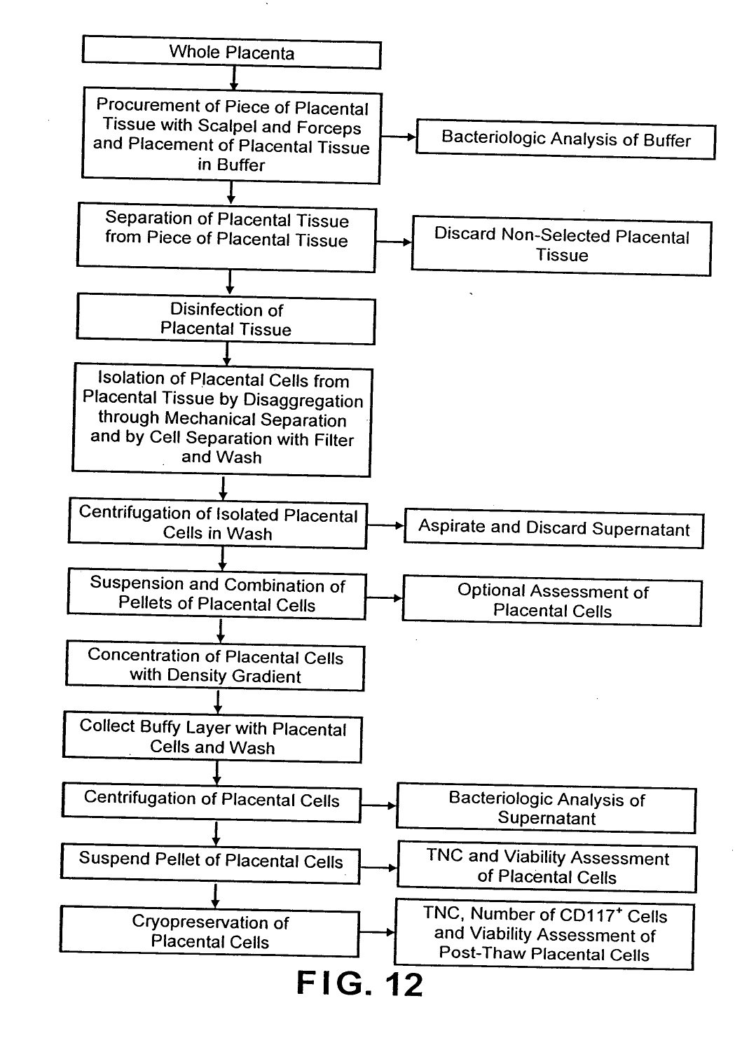

Another alternative embodiment of the present invention illustrated in FIG. 12 generally involves obtaining placental tissue with scalpel and forceps and disaggregating placental tissue by mechanical separation of the placental tissue to release placental cells comprising maternal placental stem cells. The placental cells comprising maternal placental stem cells are isolated from the placental tissue through cell separation with a filter and wash. The placental cells comprising maternal placental stem cells are collected with a density gradient in a buffy coat layer that is collected and concentrated through centrifugation steps and optionally cryopreserved in the vapor of liquid Nitrogen. Such cryopreservation may occur at or below a temperature of about −135° C.

The aforementioned embodiments of the present invention as referenced FIGS. 4 through 12 are described in more detail as provided hereinafter under the following headings Collecting, Labeling and Transporting Placental Tissue; Initial Processing of Placental Tissue at Processing Facility; Microbiological Quality Control at Pre-Processing of Sample; Disinfection of Placental Tissue; Preparation for Disaggregation of Placental Tissue; Disaggregation by Mechanical Separation; Disaggregation by Enzymatic Digestion; Centrifugation of Placental Cells; Concentration of Placental Cells by Centrifugation; Concentration of Placental Cells with Density Gradient for Cryopreservation; Preparation for Cryopreservation; Cryopreservation; and Flow Cytometry Analysis.

Collecting, Labeling and Transporting Placental Tissue

A placenta is collected and treated after vaginal or caesarean delivery in preparation for shipment to a processing facility. A maternal blood sample and cord blood sample may be collected and used as reference samples and subject to analysis to identify any blood borne infectious disease. A placenta tissue collection kit may be provided to collect, treat and package a piece of placenta for shipment. The placental tissue collection kit comprises a placental tissue transport container such as a box, bag or other container suitable for shipping a piece of placental tissue preferable at a cold temperature; a sterile tissue container of suitable size including, but not limited to, a 500 ml container; Dulbecco's Phosphate Buffer Saline (DPBS) (Mediatech or other suitable manufacturers) that contains no calcium, magnesium or phenol red; plastic zipped bags with absorbent towels; sterile scalpel; sterile forceps; sterile ruler; tincture of Iodine; and sterile 4×4 gauze; sterile basin, and sterile gloves. Alternatively, and in place of the sterile scalpel, a sterile punch biopsy (for example, a punch biopsy of 2 mm, 4 mm, 6 mm, 8 mm or other suitable size) is provided for performing punch biopsies with the placental tissue collection kit. The placental tissue collection kit may be taken to the hospital or birthing center by the donor where the placental tissue collection is completed according to instructions in the kit. Universal precautions are used through the practice of the invention. Appropriate barrier and personal protection measures including sterile gloves are used throughout the handling of the placenta and procurement of the placental tissue.

Prior to collecting the placental tissue, the donor's identity is confirmed and assigned an accession number and specimen labels are to be implemented to identify the placental tissue in the sterile tissue container. Each specimen label must be completed with date, time, and donor information including name and identity number such as social security number or other assigned information number, an accession number, and initials of individual procuring the placental tissue. Hospital labels may be used in place of a specimen label if the specimen label is not available. The specimen label or hospital label must be adhered to the sterilized specimen container for identification purposes.

After the placenta is delivered, the whole placenta is placed in a sterile basin. The sterile container is opened and the DPBS buffer is aseptically poured into the sterile container in preparation to receive the placental tissue obtained from the whole placenta.

An area of the fetal surface of the placental disc is prepared to obtain a piece of placental tissue. The area of the fetal surface may be wiped lightly with the tincture of Iodine by placing the tincture of Iodine in the center of area to be cleaned. The tincture of Iodine is circled outwards to cover the whole area of the fetal surface to be cleaned. The Iodine remains on the placenta for about 30 seconds before the Iodine is wiped dry with the sterile gauze. Preferably, a tincture of Iodine may be used. Alternatively, Povidone-iodine or Betadine may also be used in place of the tincture of Iodine. A further alternative is to wipe an area of the fetal surface with a piece of sterile gauze. Yet a further alternative is to leave the surface of the placenta as is, as delivered.

A piece of placental tissue may be procured from the area of the placenta disinfected with the tincture of Iodine through use of the sterile scalpel and forceps of the placental tissue collection kit as shown in FIG. 1. Optionally, sterile scissors may be used in place of the sterile scalpel to procure a piece of placental tissue.

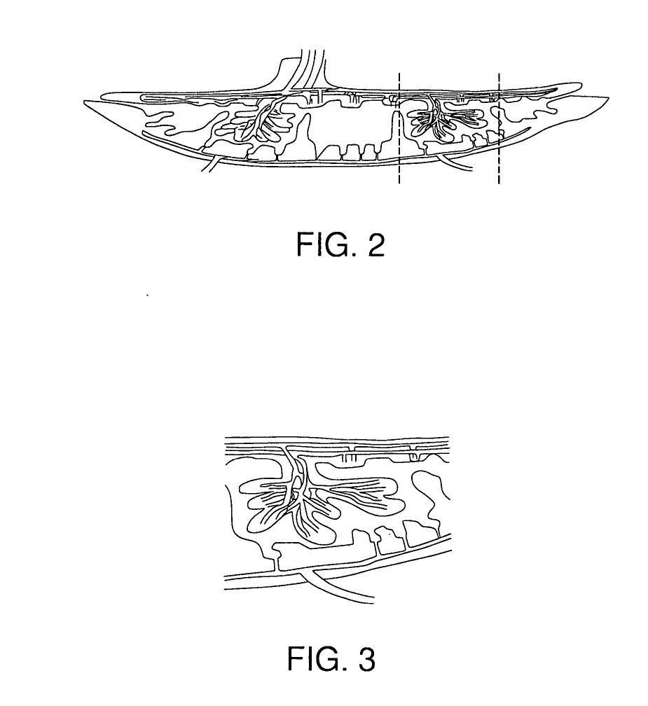

In reference to FIGS. 1 through 3, a piece of tissue of the placenta is cut away from the area of the placenta disinfected with the tincture of Iodine using the sterile ruler, scalpel, and forceps of the placental tissue collection kit. The sterile ruler is used to measure on the fetal side of the placenta, i.e., the cord side of placenta, the size of the piece of placental tissue to be cut. Once measured, a piece of placental tissue is aseptically cut using the sterile scalpel and sterile forceps as shown particularly in FIGS. 1 and 2. The size of the width and height of the piece of placental tissue may be between about 2 cm by about 2 cm to about 10 cm by about 10 cm, preferably between about 4 cm by about 4 cm to about 8 cm by about 8 cm. The width and height of the piece of placenta are measured on the fetal surface side of the placenta as shown in FIG. 1. The piece of placental tissue comprises at least maternal tissue of the placenta.

Alternatively, several punch biopsies of tissue of the placenta may be procured from the area of the placenta disinfected with the tincture of Iodine using the punch biopsy optionally provided in the placental tissue collection kit.

Many punch biopsies may be obtained from the area of the placenta disinfected with the tincture of Iodine. Punch biopsies are removed aseptically from the placenta using the sterile punch biopsy and sterile forceps. At least 10 to 20 or other suitable number of punch biopsies of placental tissue are removed from the placenta. The punch biopsy will be used repeatedly to yield the desired amount of placental tissue.

Once procured from the placenta, the piece of placental tissue as shown in FIG. 3 or punch biopsies of the placental are placed in the DPBS media in the sterilized container using the sterile forceps provided with the placental tissue collection kit. The sterilized container is tightly closed with the corresponding lid and packaged for transportation. The tissue of the placenta is to remain cold from procurement through shipment and processing at the processing facility. It is preferred that the piece of placental tissue remain at a temperature between about 1° C. to about 15° C., and preferably at a temperature between about 1° C. to about 10° C. for the duration of shipment to a suitable processing facility.

The sterilized container is packaged for transportation by placing the sterilized container in a large plastic zip bag that is sealed by closing the zip function of the bag. The large plastic bag with the sterilized container enclosed may be placed in a second large zip bag and also sealed. Four absorbent towels may be placed in the bottom of the placental tissue transport container. Two double bags full of wet ice are prepared. One of the double bags full of wet ice is placed on top of the absorbent towels. The sterilized container in the sealed bags may be placed on top of the ice bag. The second double bag full of wet ice may be placed on top of the specimen, and the remaining absorbent towels are placed on top of the second bag of ice. The placental tissue transport container is closed, secured and sealed. Other suitable containers may be used for transportation of the sterilized container as long as the container maintains the piece of placental tissue at a temperature between about 1° C. to about 15° C. and preferably between about 1° C. to about 10° C. during shipment. For example, other suitable containers include but are not limited to cooled and insulated shipping containers sold by Therapak Corporation.

A preprinted label may be provided on the placental tissue transport container next to the air bill. A carrier must be contacted within at least 2 hours of birth to pick up shipment of the placental tissue transport container. The carrier may be AirNet or other courier suitable for transporting biological materials. The placental tissue shipment container should arrive at the processing facility within about 24 hours to about 72 hours of collection. It is preferred that the placental tissue shipment container arrive at the processing facility between about 24 hours to about 48 hours after collection and particularly within about 48 hours after collection.

For multiple births, each sample of placental tissue is separately procured and processed in accordance with the invention. Each placental tissue sample is placed in its own labeled sterile container, and each sample should then be shipped with its corresponding paperwork to the processing facility in a separate placental tissue shipment container. The placental tissue shipment container may be left at room temperature prior to pick up by the courier. However, at no time should the placental tissue shipment container be stored in a hot environment.

Initial Processing of Placental Tissue at Processing Facility

The placental tissue shipment container is received at the processing facility and all donor information is obtained and inputted into the processing facility records. Once the donor information is confirmed, all tubes, batch records and blood culture bottles for microbiological detection are affixed with appropriate labels prior to processing. Appropriate barrier and personal protection measures are used throughout handling of the placental tissue and blood at the processing facility. All blood and tissue products are handled as if capable of transmitting an infectious disease. Once received at the processing facility, the sterilized container is removed from the placental tissue shipment container, placed on ice in an ice pan, and transferred into a clean room in a biological safety cabinet (BSC) for placental tissue disinfection and disaggregation and placental cell isolation, collection and preparation for cryopreservation.

Microbiological Quality Control at Pre-Processing of Sample

Aseptic techniques are used throughout the microbiological quality control at pre-processing of the sample and particularly in the BSC. In the BSC and using a sterile technique, the top of the sterilized container is removed. A labeled blood culture bottle should be visually inspected to ensure that the culture bottle is suitable for use. The visual inspection should be used to identify signs that would preclude use of the culture bottle such as turbidity, signs of gas production and/or evidence of growth (i.e. yellow sensor). Only blood culture bottles without signs of turbidity, gas production and/or evidence of growth should be used. The plastic flip top should be removed without touching the septum of the blood culture bottle. A sterile alcohol pad should be used to disinfect the septum. A new sterile alcohol pad should be opened and placed on top of the septum by touching the edges of the alcohol pad only. Using a syringe, a volume of DPBS media surrounding the procured tissue in the sterilized container may be removed and used to inoculate the labeled blood culture bottle. Alternatively, two culture bottles may be inoculated with sample for individual aerobic and anaerobic detection of bacteria. The volume of DPBS media may be between about 1.0 ml to about 4.0 ml. The blood culture bottle is incubated at about 37° C. in the automated microbiological detection system, preferably BacT/ALERT by Biomerieux or other suitable collection system so long as it is validated according to the organisms it will detect and according to manufacturer's specifications for blood culture. The BacT/ALERT blood culture bottle and system are provided as an example and not a limitation of a suitable collection system for the practice of the invention. Other suitable automated or manual blood culture specimen bottles and systems may be used as long as it is in compliance with 21 C.F.R. Section 610.12.

Disinfection of Placental Tissue

Aseptic techniques are used throughout disinfection of the placental tissue and particularly in the BSC. The placental tissue is disinfected to rid the placental tissue of contaminants. The placental tissue is subjected to a double treatment and wash for disinfection. The double treatment comprises a Betadine solution and an antibiotic dip solution. The Betadine solution may be prepared as about 50% Betadine (10% Povidone-iodine Topical wash solution—Purdue Products or other suitable solution) and about 50% PBS buffer as an initial disinfecting wash. The antibiotic dip solution may be a triple antibiotic solution, which is dependent upon tissue contaminants. For example, the antibiotic dip solution may be prepared by dissolving 1 g/vial of desiccated Cefazolin with about 10 ml HBSS, 2×1 g/vial Streptomycin with about 10 ml HBSS, 2×50 mg/vial of desiccated Amphotericin B with about 10 ml HBSS, and combining the antibiotics with HBSS to a final volume of about 150 ml. Once solubilized, the antibiotic dip solutions are removed from each vial and all of the antibiotic solutions are combined with HBSS up to a final volume of about 150 ml. The antibiotic dip solution may be stored between about 2° C. to about 8° C. for about 4 days prior to use. The double treatment is prepared by filling about 150 ml of the Betadine solution in a first sterile disposable container in the BSC, about 150 ml of the prepared antibiotic dip solution in a second sterile disposable container in the BSC, and about 150 ml of DPBS in a third sterile disposable container in the BSC.

Optionally, other antibiotic dips may be used, such as for example, antibiotic dips incorporating Cefataxim, Amikacin, Gentamycin, Vancomycin and/or Amphotericin B, similar to antibiotic dips used for decontamination of cardiac valves.

The double treatment is carried out in a BSC where the piece of placental tissue or alternatively the punch biopsies in the DPBS are removed from the sterile container and placed in a sterile Petri dish present in the BSC.

Placental tissue processing comprises selecting tissue of placenta. A varied degree of thickness of the placental tissue may be used. The full thickness of the placenta is initially harvested. By way of example, when placental tissue is selected, the tissue of the placenta is selected to include the fetal side of the placenta leaving maternal placental tissue and fetal tissue. In another embodiment, the fetal/maternal tissue interface may be dissected free from the other tissue of the placenta. A desired amount of the interface may be as thin as 1 mm. In a further embodiment, fetal placental tissue may be removed from the placental tissue and discarded so that only maternal placental tissue is processed. Placental tissue processing may occur in a BSC using sterile scissors, scalpel, and forceps.

The selected tissue of the placenta may be disinfected with a double treatment. The placental tissue is dipped and fully submerged in a Betadine solution in a first disposable container for about 5 seconds and then removed. The placental tissue is dipped and fully submerged in the antibiotic dip in a second disposable container for between about one minute to about 3 minutes and then removed. The placental tissue is dipped and rinsed in a DPBS wash in a third disposable container for between about 5 seconds to about 10 seconds and then removed. After disinfection, the placental tissue is placed in a sterile container filled with DPBS so that the placental tissue is substantially submersed in the DPBS. The sterile container is placed on ice in an ice pan for further disaggregation processing in accordance with the invention.

Preparation for Disaggregation of Placental Tissue

Aseptic techniques are used throughout isolation of placental cells from the selected placental tissue in the BSC. Throughout the process of isolating placental tissue, gloves should be wiped frequently with about 70% IPA. Gloves that are visibly contaminated with blood or reagents at anytime during the process should be discarded and replaced with new gloves. Any spills or drips should be immediately cleaned with Cavicide or another suitable EPA registered disinfectant. All materials should be wiped with about 70% IPA or Cavicide prior to placement under the BSC. The BSC should be disinfected with about 70% IPA or Cavicide before and after processing.

The manufacturer, lot number and expiration dates of all media, reagents, enzymes or solutions used in the practice of the invention are documented on the batch record. Red biohazard bags are in biohazard trash bins and sharps containers are prepared to receive discarded biological waste products and spent sharps devices. A vacuum collection system for aspirating material is assembled by attaching one end of the vacuum tubing to a pump and the other end to the “vac” port of a collection flask. Attach one end of the remaining vacuum tubing to the flask at the “patient” port. Squeeze the other end of the tubing into one of the metal slots at the side of the BSC.

A sterile stainless steel pan should be placed in the BSC for sterile supplies after the BSC has been disinfected. All sterile supplies used for the process should be placed in the pan. The sterile supplies include sterile forceps, scalpels, scissors, needles, syringes and Petri dishes. Other materials should be placed in the BSC including red top tubes, rack for red top tubes, blood culture bottles, alcohol pads, 50 ml conical collection tubes, rack for 50 ml conical collection tubes, pipettes, and ice pan. All media, enzymes and reagents are preferably placed on ice and are stored between about 2° C. to about 8° C.

Media and reagents are prepared for the isolation process. A wash solution is used throughout the placental cell isolation process. The wash solution comprises about 500 ml of HBSS with or without Calcium chloride and Magnesium chloride (Gibco), about 5 ml of Heparin (Heparin Sodium 1,000 USP Units/ml—American Pharmaceutical Partners), about 2.5 ml of DNAse (Genentech—at a concentration of 2.5 mg/ml) and about 50 ml of protein (Human Serum Albumin (HSA) 25% or equivalent—Telacris Biotherapeutics). The wash solution is placed in a container that is on ice in an ice pan in the BSC.

The disinfected placental tissue may be removed from the DPBS in the container using sterile forceps and placed on a sterile Petri dish containing wash solution. Any blood vessels or fibrous tissue present on the placental tissue may be removed from the placental tissue. Fibrous tissue, such as amniotic tissue, may optionally be left attached to the placental tissue for processing. The placental tissue may be chopped, cut and minced into small pieces using a sterile scalpel and forceps. The reduction of the placental tissue to small pieces complements the disaggregation of the placental cells from the placental tissue using either mechanical separation or enzymatic digestion.

Disaggregation by Mechanical Separation

In preparation for mechanical separation, sterile 50 ml conical tubes are labeled and placed in a sterile tube holder in the BSC. Sterile cell strainers are placed in the BSC. Prior to use, each cell strainer is removed from its packaging and is only handled through contact with the handle of the cell strainer while taking special care to avoid contact with the filter of the cell strainer. When moving the cell strainer, gloves must be confirmed to be sterile or have immediately been cleaned with IPA. A cell strainer may be a BD Falcon Cell Strainer (100 micron Nylon cell strainer) or other suitable strainer. A conical tube will be donned with a cell strainer applied as a cover to the tube.

Pieces of minced placental tissue are removed from the Petri dish and placed in the cell strainer and washed with wash solution using a sterile pipette. A sterile syringe is removed from a package by grasping the luer end to retain sterility of the handle. The handle of the syringe will be used to assist in tissue disaggregation by forcing the handle against the placental tissue in the cell strainer. The force placed against the placental tissue will act to disaggregate the placental tissue and release placental cells, which are forced through the pores of the filter of the cell strainer by force from the handle. Wash solution may be periodically placed on the placental tissue to wash placental cells through the cell strainer. The placental tissue may also be squeezed with forceps while in the cell strainer to release cells into the cell strainer, and the cells are then either forced or washed through the cell strainer. Additional placental tissue may be added to the cell strainer via sterile forceps for processing. These steps may be repeated until all of the minced placental tissue is disaggregated and forced against the cell strainer in order to obtain a maximum yield of placental cells from the substantially placental tissue in the conical collection container. When a conical collection container is filled to about 45 ml, the cell strainer is moved to the next conical collection container to continue the process until all of the minced placental tissue is disaggregated. When the cell strainer becomes clogged with debris, the cell strainer is replaced with a new cell strainer. All wash solution in the Petri dish is removed with a 10 ml pipette and rinsed through the cell strainer to collect cells in the collection container. Each conical collection container is placed on ice after it is filled with wash solution and the cells that have been forced through the cell strainer.

Alternatively, mechanical disaggregation may occur by other suitable manual and automated mechanical disaggregation systems or other suitable system adapted for releasing placental cells from placental tissue. For example, the BD Medimachine™ automated, mechanical disaggregation system may be automated to mechanically disaggregate pieces of substantially maternal placental tissue up to about one cm³ with a volume of wash solution and a filter membrane with about 100 μm pore size in accordance with the manufacturer's instructions for disaggregation. The placental cells are collected in collection tubes and subjected to centrifugation to concentrate the placental cells.

Disaggregation by Enzymatic Digestion

An enzyme is prepared for enzymatic digestion of the minced placental tissue. A suitable enzyme is collagenase or other suitable enzyme. Collagenase may be reconstituted by adding about 10 ml of cold HBSS to every 500 mg of desiccated collagenase to reconstitute the enzyme and create a final enzyme concentration of about 50 mg/ml. The HBSS and collagenase solution should be swirled gently for mixing. The solution should not be shaken vigorously to avoid degradation of the enzyme. The creation of bubbles during reconstitution of collagenase should be avoided. If the enzyme is prepared and frozen, obtain one vial about 30 minutes prior to use and allow it to thaw in an ice bath protected from light exposure.

The collagenase solution is combined with HBSS to create a digestion solution. The digestion solution is a mixture of collagenase solution and HBSS so that the collegenase is at a concentration capable of digesting the connective tissue of the placental tissue. For example, a suitable concentration of collegenase is one mg/ml of digestion solution. The digestion solution can be prepared by mixing about one ml of collagenase solution with HBSS up to a total volume of about 50 ml. The digestion solution at a volume of about 25 ml is placed in each labeled 50 ml conical collection tube. DNAse is added to the digestion solution in each conical collection tube, preferably at a volume between about 5 drops to about 10 drops per tube. DNAse is added using the sterile dropper where the DNAse is at a concentration of about 2.5 mg/ml. A volume of about 0.25 ml of preservative free Heparin (Heparin Sodium 1,000 USP Units/ml—American Pharmaceutical Partners) is added to each 25 ml of the digestion solution in each conical collection tube to prevent clotting if placental blood is still present. The final concentration of the Heparin in the digestion solution is about 10 units/ml. A sufficient amount of conical collection tubes are labeled and filled with digestion solution depending upon the amount of placental tissue that will be enzymatically digested and collected.

The small pieces of placental tissue should be of a suitable size so the enzyme will be able to gently break down the collagen in the connective tissue of the placental tissue to free the placental cells. The placental tissue must be chopped into approximately about one cm³ sizes. However, the placental tissue may be chopped into pieces that are smaller than about one cm³ and even larger than about one cm³. Placental tissue is placed into each conical collection tube containing about 25 ml of digestion solution until the volume of each conical tube is about 45 ml.

The conical collection tubes containing the digestion solution and placental tissue may be placed in a 37° C. incubator for an appropriate amount of time to digest the connective tissue of the placental tissue to release the placental cells. For example, the conical collection tubes are incubated for about 30 minutes for enzymatic digestion. The tubes may be inverted a few times at intervals during the incubation period. If it appears that the placental tissue has not undergone sufficient digestion, the enzymatic digestion may continue in the incubator for additional time, up to about one hour at about 37° C.

Once a sufficient amount of enzymatic digestion has occurred, the enzymatic reaction is stopped by adding an appropriate amount of protein to inactivate the enzyme. For example, the enzymatic digestion may be stopped by adding 25% Human Serum Albumin to the digestion solution and digested placental tissue in the conical collection tube. About 5 ml of HSA is added to each tube to neutralize the enzyme. The conical collection tube may be inverted several times to mix the HSA with the digestion solution. Once well mixed, the conical collection tubes are centrifuged to wash out the enzyme. The conical collection tubes are centrifuged at about 1400 rpm (420 g) for about 7 minutes within a range of about 2° C. to about 30° C. After the conical collection tube is removed from the centrifuge, the supernatant above the pellet is aspirated with suction while avoiding the pelleted cells. The pelleted cells in the bottom of the conical collection tube are re-suspended in a solution such as DPBS, DMEM or other suitable solution. Once re-suspended, the cells may be optionally filtered through a cell strainer and rinsed with wash solution into 50 ml conical collection tubes. Each conical collection tube is placed on ice.

Explant Methodology

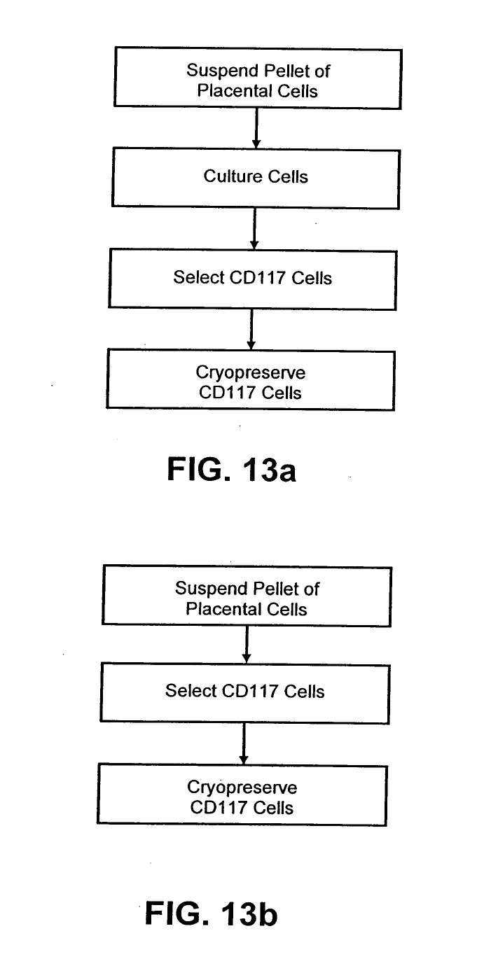

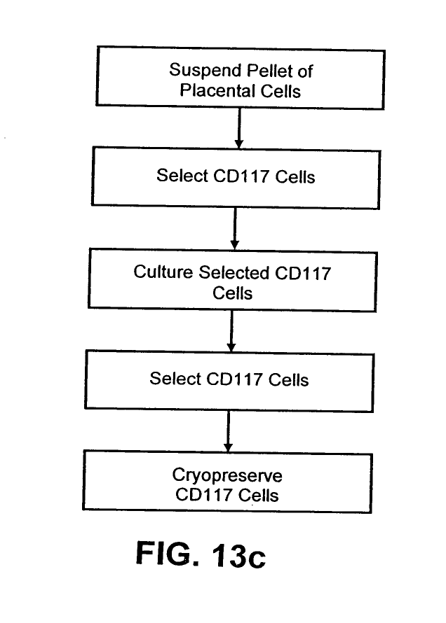

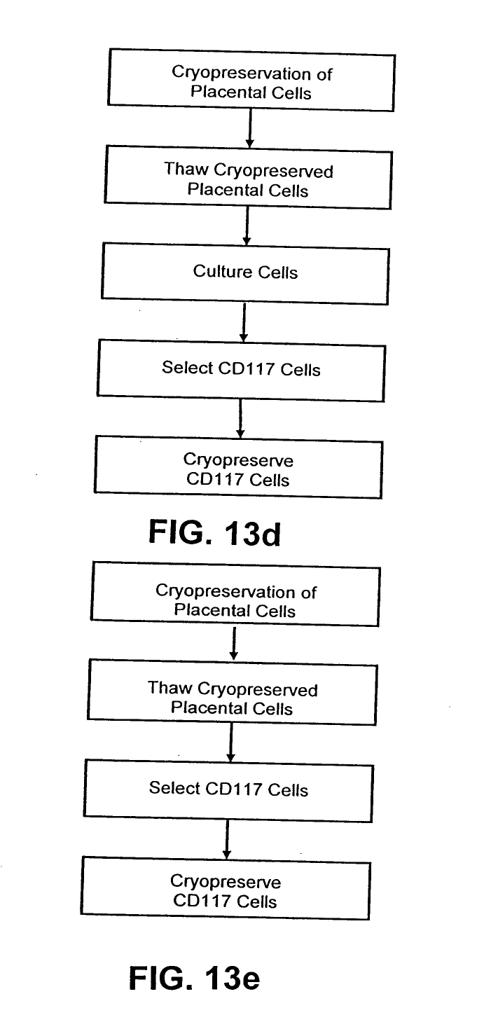

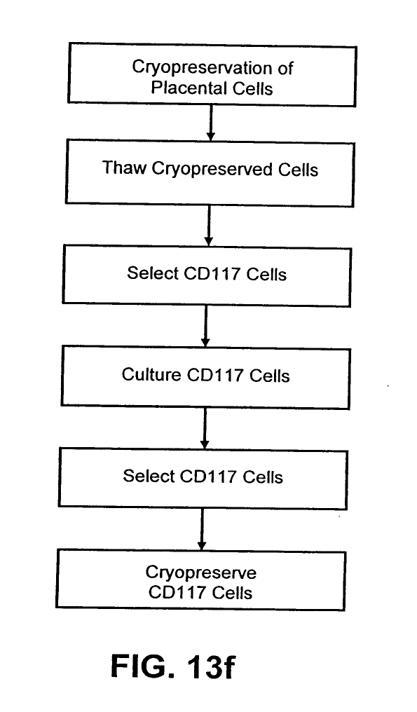

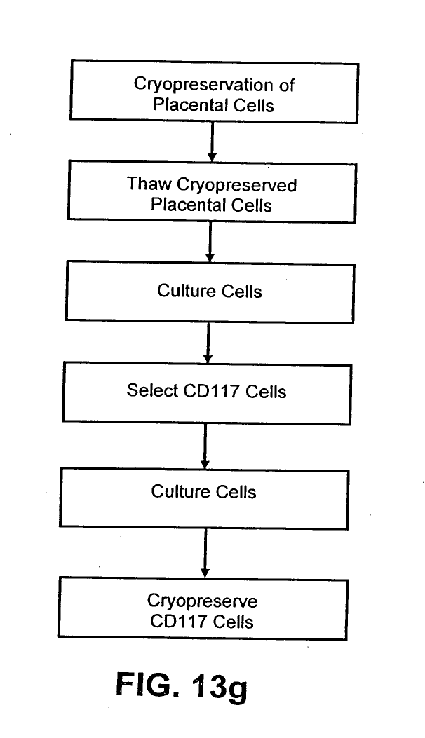

As an alternative to disaggregation by mechanical separation or enzymatic digestion, explant methodologies may be used to collect placental cells from the placental tissue. For example and rather than mincing the placental tissue, the placental tissue may be cut with a sterile scalpel or scissors along the maternal portion of the placenta to obtain one or several pieces of maternal placental tissue about 2 mm². The squares of placental tissue would be placed in an untreated tissue culture flask and allowed to dry and adhere to the flask. The time for allowing the squares of tissue to dry and adhere may be about 30 minutes to about 60 minutes. Then, tissue culture complete media, such as for example Chang's complete media would be added to the flask containing the adhered squares of placental tissue. The flask would be incubated for about 7 days to about 28 days at about 37° C. at 5% CO₂ in an incubator at which time the placental cells would leave the pieces of square placental tissue and adhere to the flask. The cells should become visible around the square pieces of placental tissue. Cell culture passages would be routinely performed according to the cell culture passage methods of the present invention. The cell cultures may be dissociated from the flask using Trypsin and according to the methods described herein. The cells collected from the cell culture may be concentrated according to the centrifugation methods of the present invention. The cells collected from the cell culture may also be cryopreserved, cultured and/or immunoselected according to the methods of the present invention, such as for example, by the methods described in FIGS. 13a through 13g, or any other suitable methods.

Centrifugation of Placental Cells

The placental cells in the wash solution collected in conical collection tubes from either mechanical separation or enzymatic digestion are subjected to centrifugation to concentrate the placental cells. The wash solution containing cells are equally separated into conical tubes for centrifugation. Centrifugation comprises subjecting the placental cells suspended in the wash solution to about 2000 rpm for about 7 minutes between about 2° C. to about 30° C. The conical collection tubes are removed from the centrifuge and the supernatant is aspirated with suction and discarded. A suitable volume of wash solution is used to re-suspend the pelleted cells in each tube. For example, about 2 ml to about 3 ml of wash solution may be used to re-suspend the pelleted cells. All of the re-suspended placental cells in the wash solution are transferred into a 50 ml conical collection tube, and the volume of the cellular suspension in the conical collection tube is brought up to a total volume of about 31 ml with the wash solution. About one ml of the cellular suspension is removed and placed in a pre-processing collection tube for testing the total nucleated cell count and viability if to be assessed with Trypan blue or other suitable viability testing method. The about 30 ml of cellular suspension may be optionally filtered through a cell strainer to remove any residual debris such as disaggregated connective tissue or unwanted cells such as red blood cells from the cellular suspension. The cellular suspension may be subjected to centrifugation and/or density gradient to collect the placental cells in the cellular suspension.

Concentration of Placental Cells by Centrifugation

The about 30 ml of cellular suspension are concentrated through centrifugation. The suspension may be centrifuged in the conical collection tube at about 2000 rpm for about 7 minutes at between about 2° C. to about 30° C.

Bacteriological analysis of the supernatant is performed using the BacT/ALERT system or other suitable system for microbial analysis. The plastic flip top is removed from the culture bottle without touching the septum of the culture bottle, which is disinfected with a sterile alcohol pad. A new sterile alcohol pad is opened and placed on top of the septum of the culture bottle. A sterile syringe used to collect between about one ml to about 4 ml of supernatant from the conical collection tube after centrifugation and to inoculate the BacT/ALERT blood culture bottle with the supernatant. Alternatively, two culture bottles may be inoculated with sample for individual aerobic and anaerobic detection of bacteria. The BacT/ALERT blood culture bottle is incubated at about 37° C. in a BacT/ALERT system in accordance with manufacturer's instructions. The remaining supernatant is aspirated with suction while carefully avoiding the pelleted cells at the bottom of the conical collection tube.

The pellet is re-suspended with wash solution up to about 6 ml to form a population of cells comprising maternal placental cells in a suspension. About one ml of cellular suspension is removed and placed into a post-processing tube and shall be tested for the total nucleated cell count, cell viability and flow cytometric analysis for CD117. About 5 ml of suspension is further processed in preparation for cryopreservation. Alternatively, a 10, 20 or 30 ml suspension may be cryopreserved from each piece of tissue.

Concentration of Placental Cells with Density Gradient for Cryopreservation

The placental cell preparation in about 30 ml of cellular suspension may be concentrated using a density gradient. A density gradient of about 15 ml is placed at the bottom of the conical tube containing the about 30 ml of cellular suspension so that the cell suspension is above the density gradient. For example, the density gradient may be Lymphocyte Separation Medium (Density 1.077-1.083 g/ml at about 20° C.—Cellgro) or other suitable gradient which may have a higher or lower density. The conical collection tube containing the cellular suspension and the density gradient is subject to centrifugation. For example, the conical collection tube may be centrifuged at about 1,400 rpm for about 30 minutes between about 15° C. to about 30° C., preferably at about 20° C., and without a brake applied to slow the centrifuge.

As a result of density gradient centrifugation, a buffy coat layer may form at the interface between the supernatant and the density gradient. The buffy coat layer contains the desired cellular population obtained from the placental tissue. The supernatant above the buffy coat layer is aspirated to about 5 ml above the buffy coat layer and discarded. The buffy coat layer is removed by gently swirling the buffy coat layer and reaming the sides of the conical tube at the buffy coat layer with a pipette. The smallest volume of density gradient as possible should be collected with the buffy coat layer. The remaining density gradient and pellet containing red blood cells is discarded. The density gradient separation should always be performed with reagents and cells at room temperature.

The buffy coat layer is placed in a 50 ml conical collection tube and the volume is brought up to about 30 ml with wash solution. The cellular suspension is subject to centrifugation at about 2000 rpm for about 7 minutes between about 15° C. to about 30° C.

Bacteriological analysis of the supernatant is performed using the BacT/ALERT blood culture bottle. The plastic flip top is removed from the culture bottle without touching the septum, which is disinfected with a sterile alcohol pad. A new sterile alcohol pad is opened and placed on top of the septum of the culture bottle. A sterile syringe is used to collect one ml of supernatant from the conical collection tube after centrifugation and to inoculate the BacT/ALERT blood culture bottle with the supernatant. Alternatively, two culture bottles may be inoculated with sample for individual aerobic and anaerobic detection of bacteria. The BacT/ALERT blood culture bottle is incubated at about 37° C. in a BacT/ALERT system according to manufacturer's specifications.

The remaining supernatant is aspirated with suction while carefully avoiding the pelleted placental cells. The pellet is resuspended with wash solution up to about 6 ml to create a population of placental cells comprising maternal placental stem cells in a suspension. About one ml of suspension is removed and placed into a post-processing tube for flow cytometry analysis as described below. The post-processing sample will be tested for the total nucleated cell count. About 5 ml of cellular suspension may be diluted up to a 10 or 20 ml suspension for further processed in preparation for cryopreservation.

Preparation for Cryopreservation

The about 5 ml of cellular suspension comprising maternal placental cells obtained by either density gradient concentration or centrifugation is combined with a cryopreservation agent in preparation for cryopreservation. The cryopreservation agent comprises a buffer, a protein, and a preservative. For example, the cryopreservation agent is about 5 ml solution comprising about 3 ml of the buffer DPBS, about one ml of the protein HSA (Telacris Bio), and about one ml of the preservative DMSO (99% Stemsol). Optionally, the DMSO concentration may be used in about 5% to about 10% concentration dependent upon the process validation. Alternatively, sterile filtered autologous plasma from the corresponding mother collected from the donor may replace the protein and buffer. Moreover, the cryopreservation agent may comprise DMSO/Dextran 40. The cryopreservation agent may be made by first combining the desired volume of DPBS and HSA and chilling the mixture for about 10 minutes on ice, and then adding about one ml of DMSO and chilling for about 10 minutes on ice. The cryopreservation agent is carefully added to the about 5 ml of cellular suspension to a total volume of about 10 ml of mixture of cellular suspension and cryopreservation agent. The mixture may be separated into desired aliquot volumes in several vials adapted for cryopreservation or maintained in one volume in one tube or may, alternatively, be separated into any other container designed for cryopreservation. For example, the mixture is separated into a 5 ml bar coded cryovials and five separate one ml QC vials adapted for cryopreservation.

Cryopreservation

The mixture of a population of cells comprising maternal placental stem cells and cryopreservation agent in the cryopreservation vials is subjected to several temperature reduction steps to reduce the temperature of the population of cells comprising maternal placental cells to a final temperature of about −90° C. utilizing a controlled rate freezer. Suitable control rate freezers include, but are not limited to, Planar Controlled Rate Freezer Kryo 10/16 (TS Scientific), Cryomed Thermo Form a Controlled Rate Freezer 7454 (Thermo Electron, Corp.). The following temperature reduction steps may be programmed in the controlled rate freezer first reducing the mixture of the population of cells and cryopreservation agent to about 4° C. and then reducing the mixture at about 1° C. per minute to about −3° C., and then about 10° C. per minute to about −20° C., and then about 1° C. per minute to about −40° C., and finally about 10° C. per minute to about −90° C. Alternatively, a program may be used that reduces the temperature of the mixture by approximately 1 to 2° C. per minute. The cryovials containing the mixture of the population of cells and cryopreservation agent are placed in the controlled rate freezer and subjected to the temperature reduction steps. Once the mixture and cryopreservation agent reaches about −90° C., the cryopreservation vials are transferred to a cryogenic storage unit and stored in the vapor of liquid Nitrogen at a temperature at or below about −135° C. For example and not a limitation, a suitable cryogenic storage unit includes, but is not limit to, LN2 Freezer MVE 1830 (Chart Industries).

Flow Cytometry Analysis

The pre-processing samples, post-processing samples and any other samples containing cells may be collected and tested for the total nucleated cell count, cell viability by Trypan blue via dye exclusion and any cell surface markers, for example and not a limitation CD117.

The total nucleated cell count may be quantified by an automated hematology analyzer (Sysmex XE-2100), by hand count with a hemocytometer or by any other means suitable for obtaining cell count.

A flow cytometry analysis of a fresh or thawed post-processing sample may be performed by spinning the mixture of the population of cells and cryopreservation agent in a vial in a centrifuge at about 2000 RPM for about 7 minutes between about 15° C. to about 30° C. until complete or, alternatively, between about 2° C. to about 30° C. After centrifugation, the supernatant is pipetted off and the pellet comprising placental cells is diluted in a wash solution. If the population of cells were frozen and need to be thawed the vials may be agitated in a 37° C. water bath and mixed by inversion while avoiding a complete thaw. Quality control may be performed to assess the total number of nucleated cells (TNC), the number of cells expressing the cell surface marker CD117 or any other cell surface marker, and cell viability using 7AAD. Cells expressing the cell surface marker CD117 may be assessed by flow cytometry using a monoclonal antibody against CD117 with a fluorescent label. For example, suitable monoclonal antibodies include, but are not limited to, BD Pharmingen PE antihuman CD117 (YB5.B8) and CD117-PE (104D2D1) or CD117-PE (95C3) both from Beckman Coulter. Further TNC cell recovery will be calculated in accordance with the invention.

Accurately pipette approximately between about 0.5 million cells to about 10.0 million cells of a well-mixed sample of the population of cells comprising maternal placental stem cells into two tubes and vortex briefly.

Wash the cells in the two tubes with about one ml of wash solution and centrifuge in Blood Bank Serofuge for about one minute. Decant supernatant without disturbing the pellet comprising placental cells. Add about 100 uL of wash solution with a micropipetter back into each tube. Vortex briefly. In the tube for testing, add from about 5 ul to about 10 uL of CD117-PE dependent upon assay validation, about 20 uL of CD44-FITC, about 10 uL of CD45-ECD, and about 20 uL of 7-AAD Viability dye. In the tube for isotype control, add about 5 uL to about 10 uL of IgG-PE dependent upon assay validation, about 20 uL of IgG-FITC, about 10 uL of IgG-EDC and about 20 uL of 7-AAD Viability dye. Incubate the tubes at room temperature (between about 15° C. to about 30° C.) for about 20 minutes while protecting from light exposure. If sample contains red blood cells with a hematocrit of greater than 5%, lyse for about 10 minutes and protect from light. However, if sample was collected by density gradient or thawed, do not lyse sample. Program carousel work list on FC500 instrument or equivalent. If sample was not lysed, wash after about 20 minutes incubation with about one ml of wash solution and decant supernatant. If sample was lysed, serofuge sample and decant supernatant. Add wash solution up to about one ml and spin again and decant supernatant. Add about one ml of Sheath fluid, vortex and run on FC500 or other suitable flow cytometer.

The CD117 positive cell count and cell viability may be reported from the flow cytometry reports and transcribed to the work documents for thawed post-processing samples. The post sample includes the isotype control result which will be subtracted, if applicable, from the total post CD117 count and documented on the CD117 post report. The instrument used to run post sample will be recorded on the work document. Results should be assessed for discrepancies between post-processing values and discrepant samples should be repeated before reporting.

Calculations

The total nucleated cell count (TNC) will be converted as TNC (×10³/uL) to TNC (×10⁶/ml). Pre-count TNC equals TNC (×10⁶/ml)×Volume (ml).

Data Collection

All collected processing information may be recorded in the batch record for each sample of placental tissue and may be bound by day in a folder including batch record, cell count data worksheets, CD117 viability worksheets and Freezerworks or other suitable inventory management and planar record system.

Current Good Manufacturing Practice (cGMP) standards and current Good Tissue Practice (cGTP) established by the United States Food and Drug Administration in the Code of Federal Regulations are followed throughout the practice of all embodiments of the present invention.

The aforementioned steps of the processes, methods, and systems of the present invention may be alternatively embodied as illustrated by FIGS. 4 through 12.

### Example 1A

A whole mammalian placenta was delivered and a tincture of iodine was placed on the fetal side of the placenta in accordance with the invention. A piece of placental tissue about 4 cm by 4 cm wide and cut through the depth of the organ from the fetal side to the maternal side was obtained from the whole placenta through use of the sterile scalpel and forceps provided in a tissue collection kit. The DPBS of the tissue collection kit was poured into the sterile container of the tissue collection kit and the piece of placental tissue was submerged in the DPBS. The sterile container was closed, chilled and packaged for shipment in accordance with the present invention and then shipped to the processing facility. The piece of placental tissue arrived at the processing facility between about 24 hours to about 48 hours after collection of the placental tissue. The sterile container with the tissue sample was received at the processing facility and unpackaged from the shipping box and logged into the batch record.

The sterile container containing the DPBS and piece of placental tissue was disinfected, transferred into a clean room, and placed on ice in an ice pan. The sterile container was placed in a BSC and the top of the sterile container was removed. A sample of the DPBS buffer (about 4 ml) was removed from the sterile container using a sterile syringe and used to inoculate an aerobic BacT/ALERT blood culture bottle to test for bacteria and fungal contamination. The blood culture bottle was incubated at about 37° C. in a BacT/ALERT system for about 7 days, whereby the results indicate a positive identification for coagulase negative Staphylococcus spp., as shown in Table 1.

TABLE 1
BacT/ALERT System Analysis

| Status | Type | Loaded | Cell |
|---|---|---|---|
| POSITIVE - COAGULASE NEG. | BTA PF | 5/12/06 @ 15:01 | 2A07 |
| STAPHYLOCOCCUS SPECIES | | | |

The piece of placental tissue was removed from the sterile container for decontamination. Prior to disinfection, the fetal placental membrane of the piece of placenta was physically separated from portions of the maternal membrane of the piece of placenta using forceps and a scalpel, and portions of the maternal piece of the placenta was discarded. Placental tissue from the fetal side to approximately 50% of the depth of the organ was removed and used for processing.

The placental tissue was then subjected to a disinfection process in the BSC. The placental tissue was initially disinfected by dipping it into a container of about 150 ml of about 50% mixture of Betadine (10% Povidone-iodine Topical Solution—Purdue Products) and about 50% PBS buffer for about 5 seconds and removed. The placental tissue was then disinfected by dipping it in a container of about 150 ml of a solution of several antibiotics to kill a broad range of pathogens for about 3 minutes. The antibiotic solution comprised three antibiotics including Amphotericin B, Streptomycin, and Cephazolin (X-Gen Pharmaceuticals Inc.) mixed with a buffer HBSS. The antibiotic solution was prepared by suspending about 50 mg of each antibiotic in about 10 ml of HBSS. The suspended antibiotics were added to about 100 ml of HBSS. Finally, the placental membrane was rinsed by dipping it in DPBS (1× without Calcium and Magnesium—Cellgro) for about 15 seconds.

The disinfected placental membrane was then transferred to a sterile container with DPBS (1× without Calcium and Magnesium—Cellgro). All of the steps of tissue separation and decontamination were performed at room temperature of about 18° C.

The sterile container with disinfected placental tissue was placed on ice in preparation for performing the subsequent steps of the invention.

The placental tissue was removed from the sterile container and placed in a wash solution in a Petri dish. The wash solution comprised about 500 ml of HBSS without Calcium chloride and Magnesium chloride (Gibco), about 5 ml of Heparin (Heparin Sodium 1,000 USP Units/ml—American Pharmaceutical Partners), about 2.5 ml of DNase (Genentech 2.5 mg/ml), and about 50 ml of protein (25% Human serum albumin HSA—Telacris Biotherapeutics). The wash solution was chilled on ice. The placental tissue in the wash was minced with a scalpel and forceps to the smallest possible pieces, which were suspended in the wash solution in a Petri dish.

Each piece of minced placental tissue was placed in a cell strainer associated with a 50 ml conical collection tube. Each piece of minced tissue was forced against a cell strainer using the end of a plunger of a syringe opposite the luer end. The minced tissue was disaggregated manually and placental cells were forced through the cell strainer and into the underlying conical collection tube. The cell strainer was a BD Falcon Cell Strainer (100 micron Nylon cell strainer). As each piece of minced tissue was removed from the Petri dish and placed in the cell separator, it was washed with the wash solution and manually forced against the cell strainer and washed again with wash solution. The placental tissue was also squeezed with forceps to release additional placental cells into the cell strainer, and the cells were then either forced or washed through the cell strainer. When a cell strainer became clogged with pieces of tissue, it was replaced with a new cell strainer. When a 50 ml conical collection tube became filled with placental cells and wash solution, the cap was placed on the conical collection tube, which was then placed on ice. Additionally, the cell strainer was placed on an empty 50 ml conical collection tube and the process of disaggregation was continued.

These steps were repeated in order to obtain a yield of a population of cells from the placental tissue in 50 ml conical collection tubes. All pieces of minced placental tissue were manually forced against the cell strainer, and all wash in the Petri dish was rinsed through the cell strainer to collect placental cells in the conical collection container. The conical collection container was placed on ice after it was filled with wash and cells that had been forced through the cell strainer.

The yield of the wash containing cells filled ten 50 ml conical collection tubes that were subjected to centrifugation. Centrifugation was performed at about 2000 rpm for about 7 minutes at a temperature of about 18° C. The conical collection tubes were removed from the centrifuge, and the supernatant was aspirated with suction and discarded while avoiding the disruption of the pelleted cells formed at the bottom of each conical collection tube. About 2 ml to about 3 ml of wash solution was used to suspend the cells of each pellet in each conical collection tube. All of the suspended cells were transferred to a 50 ml conical collection tube and the volume of the cellular suspension was brought up to a total volume of about 31 ml using the wash solution. A pre-processing sample was collected by removing about one ml of the cellular suspension and placing it in an analysis tube for flow cytometry analysis. The remaining about 30 ml of the cellular suspension was concentrated by density gradient centrifugation.

The remaining about 30 ml of cellular suspension was underlayed with a density gradient of about 15 ml of Lymphocyte Separation Medium (Density 1.077-1.080 g/ml at about 20° C.—Cellgro). Alternatively, the about 30 ml of cellular suspension could have been overlaid on top of the density gradient. The LSM was placed at the bottom of the tube containing the about 30 ml of suspension so that the cellular suspension was above the density gradient.

The 50 ml conical collection tube containing the cellular suspension and the density gradient was subjected to centrifugation at about 14,000 rpm for about 30 minutes without a brake at a temperature at about 20° C. It is preferred that this step of centrifugation occur between about 20° C. to about 24° C. A buffy coat layer formed at the interface between the supernatant and the density gradient. The buffy coat layer contained the desired cellular population of placental cells expressing CD117. The supernatant above the buffy coat layer was aspirated and discarded. The buffy coat layer was removed along with some wash solution and as little volume of density gradient as possible. The remaining density gradient and pellet containing red blood cells was discarded along with the 50 ml conical collection tube.

The buffy coat layer was placed in a 50 ml conical collection tube and the volume was brought up to about 30 ml with wash solution. The cellular suspension was subjected to centrifugation at about 2000 rpm for about 7 minutes at room temperature of about 20° C. It is preferred that this step of centrifugation occur at a temperature between about 20° C. to about 24° C. After centrifugation, about one ml of the supernatant near the top of the conical collection tube was removed and used to inoculate a BacT/ALERT blood culture bottle to test for bacteria and fungal contamination. The blood culture bottle was incubated at about 37° C. in a BacT/ALERT system for about 7 days, whereby the results were negative as shown in Table 2.

TABLE 2
BacT/ALERT System Analysis

| Status | Type | Loaded | Cell |
|---|---|---|---|
| NEGATIVE | BTA PF | 5/12/06 @ 15:01 | 2A08 |

The remaining supernatant was aspirated while carefully leaving the pellet containing the placental cell population expressing the cell surface marker CD117. The pellet was suspended in wash solution up to about 6 ml. About one ml of cellular suspension was collected as a post-processing sample and placed in an analysis tube for flow cytometry to obtain cell count, flow cytometry analysis and cell viability. About 5 ml of the cellular suspension was further processed in preparation for cryopreservation.

The placental cell preparation in about 5 ml of cellular suspension obtained by density gradient centrifugation was combined with a preservation agent, such as a cryopreservation agent, in preparation for cryopreservation. The cryopreservation agent comprised buffer, protein and preservative. The cryopreservation agent was about 5 ml solution comprising about 3 ml of the buffer DPBS, about one ml of the protein HAS (Telacris Bio), and about one ml of the preservative DMSO (Stemsol). The cryopreservation agent was made by first combining the desired volume of DPBS and HSA and chilling the mixture for about 10 minutes on ice, and then adding about one ml of DMSO and chilling for about 10 minutes on ice.

The cryopreservation agent was carefully added to the about 5 ml of cellular suspension to a total volume of about 10 ml. The mixture was separated into about a 5 ml volume in a cryopreservation vial and about five 1 ml aliquot volumes in separate cryovials for cryopreservation.

The cryopreservation vials were subjected to several temperature reduction steps using the Planar Cryopreservation freezer to reach a final sample temperature of about −90° C. The cryopreservation vials were placed in a Planar freezer and the temperature reduction program was set for several temperature reduction steps. The mixture of the population of cells and cryopreservation agent in the cryopreservation vials was subjected to several temperature reduction steps to reduce the temperature of the mixture containing placental cells to a final temperature of about −90° C. utilizing a Planar cryopreservation freezer. The reduction steps involved first reducing the mixture to about 4° C. and then reducing the mixture about 1° C. per minute to about −3° C., and then about 10° C. per minute to about −20° C., and then about 1° C. per minute to about −40° C., and finally about 10° C. per minute to about −90° C. Once the mixture reached about −90° C., the cryopreservation vials were transferred to a cryogenic storage unit and stored in the vapor of liquid Nitrogen at a temperature at or below about −135° C.

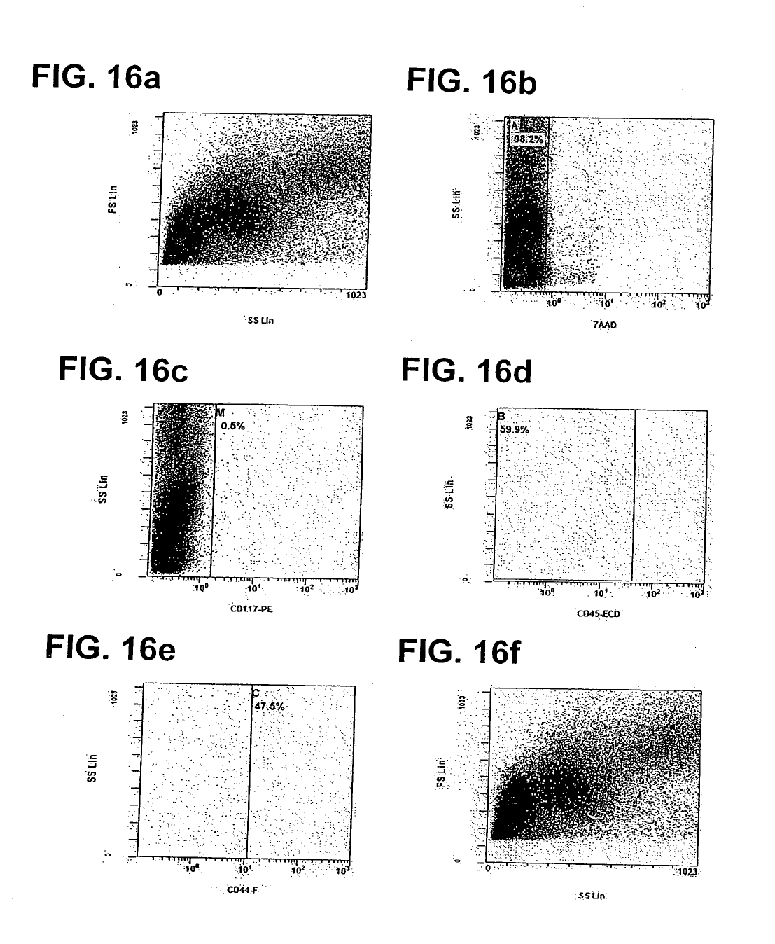

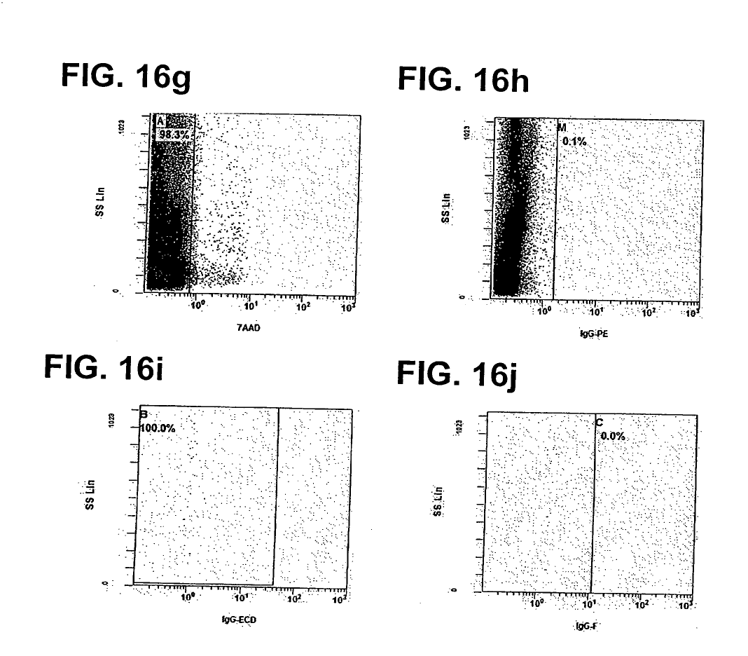

The TNC for the pre-processing and post-processing samples were performed in accordance with the present invention using an automated hematology analyzer (Sysmex XE-2100) as referenced herein. The results indicated that about 314.4 million cells were collected in the pre-processing sample. The results indicated that about 16.2 million cells were collected in the post-processing sample. Flow cytometry analysis was performed using a FC500 Flow Cytometer with the results of that analysis shown in FIGS. 16a through 16j. The results indicated that CD117⁺ cells were collected at a concentration of about 0.4% of the TNC population. Additionally, the results indicated that CD117+ CD45− cells were collected at a concentration of about 59.9% of the TNC population. Furthermore, the results indicated that CD117+ CD44+ cells were collected at a concentration of at about 47.5% of the TNC population. The results also indicate that about 98% of cells collected were viable as determined by 7AAD viability analysis.

The thawed post-processing sample comprised about one ml cellular suspension that was thawed in accordance with the post-cryopreservation thawing method of the present invention. The results indicated that about 8.6 million cells were present in the thawed post-processing sample. The thawed product was processed and diluted with wash solution in accordance with the invention and placed in an analysis tube for flow cytometry analysis. The results indicate a concentration of CD117⁺ cells at about 0.1% of the TNC population, a concentration of CD117+ CD45− cells at about 61.3% of the TNC population, a concentration of CD117+ CD44+ cells at about 33.1% of the TNC population, and about 91% cell viability as determined by 7AAD viability analysis. The results were calculated by running the post-processing sample in accordance with the flow cytometry analysis of the invention and subtracting background calculations obtained with a post-processing sample run with an isotype of an IgG antibody. Background calculations for flow cytometry analysis in the present invention were obtained and subtracted for all flow cytometry experimentation.

Study

A randomized study was performed. The randomized study population consisted of a prospective cohort of 43 donated placentae from pregnant women consenting to the donation of their placenta post-childbirth for non-clinical research studies. There was no inclusion criteria. Exclusion criteria include placentae shipped without media, placentae arriving outside of the temperature range of between about 1° C. to 15° C. and placentae greater than about 72 hours post delivery. The study randomized the first patient to be recruited for the study and then recruit every patient thereafter until the desired sample size was obtained for the study.

The independent variable procurement of tissue and isolation of placental cells from tissue and the dependent variables the number of CD117 positive placental cells, the total number of nucleated cells, the total nucleated cell recovery from a thawed sample, and the total cell viability as assessed by 7AAD were analyzed as discussed below. The independent variable of tissue procurement (scalpel and punch biopsy) and the dependent variables the number of CD117 positive placental cells, the total number of nucleated cells, the total nucleated cell recovery from a thawed sample, and the total cell viability as assessed by 7AAD were statistically assessed as discussed below. The independent variable of isolating cells from the placental tissue (enzymatic digestion and mechanical separation) and the dependent variables of the number of CD117 positive placental cells, the total number of nucleated cells, and the total cell viability as assessed by 7AAD were statistically assessed as discussed below.

Other variables may be measured for the placentae collected during the study to control for possible confounding events. The variables include time from collection to processing the placenta, temperature of the organ upon arrival, weight of the organ, weeks of gestation at delivery, whether the placenta was procured by vaginal or cesarean delivery, sex of the baby, and abnormalities on the placenta.

The analysis of the data was performed by SPSS. The primary statistical tests used to test the study hypotheses were ANOVA and MANOVA for repeated measures. The study subjects were placentae. All statistical tests conducted in the study were performed to determine the significance on paired samples. All statistical tests were tested at a level of significance of α=0.05. Aspects of the study are provided in the following Examples.

### Example 1

Punch biopsies of placental tissue from 43 individual placentae were separately collected using a sterile punch biopsy in accordance with the punch biopsy methods of the present invention. Ten punch biopsies were procured from each placenta and individually labeled, transported, and processed at the processing facility in accordance with the packaging and transportation methods of the present invention (i.e., packaged in a container to maintain the biopsies at a temperature between about 1° C. to about 10° C. for the duration of shipment, shipped to a processing facility and then received at a processing facility within about 48 hours of procurement). The punch biopsies from each placenta were processed separately from the punch biopsies of the other placentae as described hereinafter.

Punch biopsies from each placenta separate from the punch biopsies of each other placenta were subjected to microbiological quality control at pre-processing of the sample and disinfected (i.e., double treatment with Betadine and antibiotic dip solutions and washed), all in accordance with methods of the present invention.

The disinfected placental tissue biopsies of each placenta were disaggregated by mechanical separation into pieces of minced tissue. The placental cells were collected by straining the minced tissue against a cell strainer and washing the minced tissue with wash solution. The placental cells present in wash solution in a tube were centrifuged at about 2000 RPM for about 7 minutes at about 4° C. The placental cells were concentrated by centrifugation for cryopreservation. The pellets comprising maternal placental stem cells were re-suspended in wash solution and prepared for cryopreservation by combining the re-suspended placental cells with about 5 ml of a cryopreservation agent per about 5 ml of placental cell suspension according to the cryopreservation preparation methods of the present invention.

The placental cells in cryopreservation agent were cryopreserved below about −135° in accordance with the cryopreservation methods of the present invention.

Thawed post-processing samples were analyzed to collect data concerning TNC, the number of CD117 cells, and the viability of all cells collected as determined by 7AAD. The TNC per gram of tissue in the fresh/post-processing samples was 1.2699×10e6 with a standard deviation of 0.81660. The Total Cell Viability obtained by 7AAD in the thawed/post-processing samples was 0.7791 with a standard deviation of 0.04955. The Total number of CD117 cells per gram of tissue in the thawed/post-processing samples was 0.6648×10e4 with a standard deviation of 0.84880. The TNC recovery determined by comparing the fresh/post-processing samples to the thawed post-processing samples was 0.8068 with a standard deviation of 0.48891.

Fresh post-processing samples and thawed post-processing samples were analyzed to collect data concerning total nucleated cell count per gram of tissue, the number of CD117 cells per gram of tissue, and the viability of all cells collected as determined by 7AAD. The analysis was performed on cell data obtained by using manual counting methods. The TNC per gram of tissue in the thawed/post-processing sample was about 0.38×10e6 with a standard deviation of about 0.4103. The Total Cell Viability obtained by 7AAD in the thawed/post-processing samples was about 0.7706 with a standard deviation of about 0.0459. The total number of CD117+ cells per gram of tissue was about 0.24×10e4 with a standard deviation of about 0.2157.

### Example 2

A piece of placental tissue was separately procured from 43 individual placentae. Each piece of placental tissue was collected using a sterile scalpel and forceps in accordance with the tissue collection methods of the present invention. Each piece of placental tissue was procured from each placenta and individually labeled, transported, and processed at the processing facility in accordance with the packaging and transportation methods of the present invention (i.e., packaged in a container to maintain the biopsies at a temperature between about 1° C. to about 10° C. for the duration of shipment, shipped to the processing facility and then received at a processing facility within 40 hours of procurement). Each piece of placental tissue measured at about 2 cm long by about 2 cm wide and were cut through the depth of the placenta from the fetal side to the maternal side of the placenta. Each piece of placenta separate from the other pieces of placenta were processed as described hereinafter.

Each piece of placental tissue was subjected to microbiological quality control at pre-processing of sample, and portions of the fetal placental tissue were removed from the maternal placental tissue and discarded. The remaining placental tissue comprising maternal placental tissue was disinfected (i.e., double treatment with Betadine and antibiotic dip solutions and washed), all in accordance with methods of the present invention. Each disinfected piece of placental tissue was about 50% of the overall thickness of the whole placenta. Each disinfected piece of placental tissue comprised about 50% fetal tissue and about 50% maternal tissue.

Each piece of disinfected placental tissue was disaggregated by mechanical separation into pieces of minced tissue. The placental cells were collected by straining the minced tissue against a cell strainer and washing the minced tissue with wash solution. The placental cells present in wash solution in a tube were centrifuged at about 2000 RPM for about 7 minutes at about 4° C. The placental cells were concentrated by centrifugation for cryopreservation. The pellets comprising maternal placental stem cells were re-suspended in wash solution and prepared for cryopreservation by combining the re-suspended placental cells with about 5 ml of cryopreservation agent per about 5 ml of placental cell suspension.

The placental cells comprising maternal placental stem cells in cryopreservation agent were cryopreserved below about −135° C. in accordance with the cryopreservation methods of the present invention.

Fresh post-processing samples and thawed post-processing samples were analyzed to collect data concerning TNC, the number of CD117 cells, and the viability of all cells collected as determined by 7AAD. The TNC per gram of tissue in the fresh/post-processing samples was 4.0928×10e6 with a standard deviation of 2.18992. The Total Cell Viability obtained by 7AAD in the thawed/post-processing samples was 0.8142 with a standard deviation of 0.04196. The Total number of CD117⁺ cells per gram of tissue in the thawed/post-processing samples was 1.4605×10e4 with a standard deviation of 1.38990. The TNC recovery determined by comparing the fresh/post-processing samples to the thawed post-processing samples was 0.7968 with a standard deviation of 0.19290.

Thawed post-processing samples were analyzed to collect data concerning total TNC per gram of tissue, the number of CD117+ cells per gram of tissue, and the viability of all cells collected as determined by 7AAD. The analysis was performed on cell data obtained by using manual counting methods. The TNC per gram of tissue in the fresh/post-processing sample was about 1.06×10e6 with a standard deviation of about 0.6519. The Total Cell Viability obtained by 7AAD in the thawed/post-processing samples was about 0.8086 with a standard deviation of about 0.0403. The total number of CD117 cells per gram of tissue in the thawed/post-processing samples was about 0.44×10e4 with a standard deviation of about 0.3103.

### Example 3

A piece of placental tissue was separately procured from 43 individual placentae. Each piece of placental tissue was separately collected using a sterile scalpel and forceps in accordance with the tissue collection methods of the present invention. Each piece of placental tissue procured from each placenta was individually labeled, transported, and processed at the processing facility in accordance with the packaging and transportation methods of the present invention (i.e., packaged in a container to maintain the biopsies at a temperature between about 1° C. to about 10° C. for the duration of shipment, shipped to a processing facility and then received at a processing facility within about 48 hours of procurement). Each piece of placental tissue measured at about 2 cm long by about 2 cm wide and was cut through the depth of the placenta from the fetal side to the maternal side of the placenta. Each piece of placental tissue separate from the other pieces of placenta was processed as described hereinafter.

Each piece of placental tissue was subjected to microbiological quality control at pre-processing of sample, and portions of the fetal placental tissue were removed from the maternal placental tissue and discarded. The remaining placental tissue comprising maternal placental tissue was disinfected (i.e., double treatment with Betadine and antibiotic dip solutions and washed), all in accordance with methods of the present invention. Each disinfected piece of placental tissue was about 50% of the thickness of the whole placenta. Each piece of placental tissue comprised about 50% fetal tissue to about 50% maternal tissue.

Each piece of disinfected placental tissue was minced and disaggregated by enzymatic digestion (i.e., about 10 g of placental tissue suspended in about 25 ml of collegenase solution (50 mg/ml) for about 45 minutes at about 33-37° C. The digestion was stopped using about 10 ml of HSA (25%) in each tube of enzyme solution. The tubes were inverted to mix and the tissue was removed. Placental cells were collected and centrifuged at about 2000 RPM for about 7 minutes at about 4° C. The pelleted cells were re-suspended in DPBS to about 45 ml. The placental cells were concentrated by centrifugation at about 2000 RPM for about 7 minutes at about 4° C. The pellets were re-suspended in wash solution and prepared for cryopreservation by combining the re-suspended placental cells with about 5 ml of cryopreservation agent per about 5 ml of placental cell suspension according to the cryopreservation preparation methods of the present invention.

The placental cells in cryopreservation agent were cryopreserved below about −135° in accordance with the cryopreservation methods of the present invention.

Fresh post-processing samples and thawed post-processing samples were analyzed to collect data concerning TNC, the number of CD117 cells, and the viability of all cells collected as determined by 7AAD. The TNC per gram of tissue in the fresh/post-processing samples was 8.7176×10e6 with a standard deviation of 13.89071. The Total Cell Viability obtained by 7AAD in the thawed/post-processing samples was 0.8450 with a standard deviation of 0.06662. The Total number of CD117 cells per gram of tissue in the thawed/post-processing samples was 0.9159×10e4 with a standard deviation of 1.11935. The TNC recovery determined by comparing the fresh/post-processing samples to the thawed post-processing samples was 0.3496 with a standard deviation of 0.21044.

Thawed post-processing samples were analyzed to collect data concerning TNC, the number of CD117 cells per gram of tissue, and the viability of all cells collected as determined by 7AAD. The analysis was performed on cell data obtained by using manual counting methods. The TNC per gram of tissue in the thawed/post-processing sample was about 1.10×10e6 with a standard deviation of about 0.4207. The Total Cell Viability obtained by 7AAD in the thawed/post-processing samples was about 0.8453 with a standard deviation of about 0.0656. The total number of CD117+ cells per gram of tissue was about 0.60×10e4 with a standard deviation of about 0.6513.

### Example 4

Punch biopsies of placental tissue were separately procured from 43 individual placentae. The punch biopsies were collected using a sterile punch biopsy in accordance with the punch biopsy methods of the present invention. Ten punch biopsies were procured from each placenta and individually labeled, transported, and processed at the processing facility in accordance with the packaging and transportation methods of the present invention (i.e., packaged in a container to maintain the biopsies at a temperature between about 1° C. to about 10° C., shipped and then received at a processing facility within about 48 hours of procurement). The punch biopsies from each placenta were processed separately from the punch biopsies of the other placentas as described hereinafter.

The punch biopsies were subjected to microbiological quality control at pre-processing of the sample and disinfected (i.e., double treatment with Betadine and antibiotic dip solutions and washed), all in accordance with methods of the present invention. The disinfected placental tissue was minced and disaggregated by enzymatic digestion (i.e., about 10 g of placental tissue suspended in about 25 ml of collegenase solution (50 mg/ml) for about 45 minutes at 37° C. The digestion was stopped using about 10 ml of HSA (25%) in each tube of enzyme solution. The tubes were inverted to mix and the tissue was removed. Placental cells were collected and centrifuged at about 2000 RPM for about 7 minutes at about 4° C. The pelleted cells were re-suspended in DPBS to about 45 ml. The placental cells were concentrated by centrifugation at about 2000 RPM for about 7 minutes at about 4° C. The pellets were re-suspended in wash solution and prepared for cryopreservation by combining the re-suspended placental cells with about 5 ml of cryopreservation agent per about 5 ml of placental cell suspension according to the cryopreservation preparation methods of the present invention.

The placental cells in cryopreservation agent were cryopreserved below about −135° in accordance with the cryopreservation methods of the present invention.

Thawed post-processing samples were analyzed to collect data concerning TNC per gram of tissue, the number of CD117 cells per gram of tissue, and the viability of all cells collected as determined by 7AAD. The analysis was performed on cell data obtained by using manual counting methods. The TNC per gram of tissue in the thawed/post-processing sample was about 0.48×10e6 with a standard deviation of about 0.2413. The Total Cell Viability obtained by 7AAD in the thawed/post-processing samples was about 0.8056 with a standard deviation of about 0.0610. The total number of CD117+ cells per gram of tissue was about 0.15×10e4 with a standard deviation of about 0.1177.

Statistical Analysis of Examples 1 Through 4

One-way ANOVA statistical analysis was performed on the results of Example 1 through Example 4 in two areas of consideration. The first area is Between Groups and represents the variation of the group means around the overall mean. The second area is Within Groups and represents the variation of the individual scores around their respective group means. The significance level of the F-test demonstrates that group differences with a value less than about 0.05. The significance level means that at least one methodology from the group differs from another methodology of the group. ANOVA statistical analysis is shown for White Blood Cells per gram of tissue in Table 3, for Total 7AAD in Table 4, for CD117 cells per gram of tissue×10e6 in Table 5, and WBC recovery in Table 6.

TABLE 3
White Blood Cells per Gram of Tissue

| | Sum of | | | | |
|---|---|---|---|---|---|
| | Squares | df | Mean Square | F | Sig. |
| Between Groups | 1407.285 | 3 | 469.095 | 8.726 | .0000206 |
| Within Groups | 9031.540 | 168 | 53.759 | | |
| Total | 10438.826 | 171 | | | |

TABLE 4
Total 7AAD

| | Sum of | | | | |
|---|---|---|---|---|---|
| | Squares | df | Mean Square | F | Sig. |
| Between Groups | .096 | 3 | .032 | 10.131 | .0000036 |
| Within Groups | .528 | 168 | .003 | | |
| Total | .624 | 171 | | | |

TABLE 5
CD117 cells with 7AAD per gram of tissue × 10e6

| | Sum of | | | | |
|---|---|---|---|---|---|
| | Squares | df | Mean Square | F | Sig. |
| Between Groups | 28.212 | 3 | 9.404 | 8.817 | .00001839 |
| Within Groups | 179.185 | 168 | 1.067 | | |
| Total | 207.397 | 171 | | | |

TABLE 6
WBC Recovery

| | Sum | | Mean | | |
|---|---|---|---|---|---|
| | of Squares | df | Square | F | Sig. |
| Between Groups | 6.166 | 3 | 2.055 | 18.722 | .0000000002 |
| Within Groups | 18.445 | 168 | .110 | | |
| Total | 24.611 | 171 | | | |

One-way ANOVA statistical analysis was performed on the average results obtained by manual cells count. The first area is Between Groups and represents the variation of the group means around the overall mean. The second area is Within Groups and represents the variation of the individual scores around their respective group means. The significant level of the F-test demonstrates that group differences with a value of less than about 0.5. The significance level means that at least one methodology from the group differs from another methodology of the group.

In order to determine the specific group differences, Post Hoc testing was performed via LSD and Bonferroni. Post Hoc testing was performed for TNC per gram of tissue, total 7AAD, the number of CD117 cells with total 7AAD negative viable cells, and TNC recovery. Post hoc testing lists pairwise comparisons of the group means for all four procedures, namely tissue collection by punch biopsy with mechanical separation of fetal/maternal placental tissue in Example 1, tissue collection by scalpel with mechanical separation of fetal/maternal placental tissue in Example 2, tissue collection by scalpel with enzymatic digestion in Example 3, and tissue collection by punch biopsy with enzymatic digestion in Example 4. The mean difference denotes the difference between the sample means. Significance lists the probability that the population mean difference is zero. There was a 95% confidence interval for each difference. If the interval contains zero, the two groups do not differ. Significance is noted for each of the two methods compared.

### Further Embodiments of the Invention

Another embodiment of the invention illustrated in FIG. 13a generally comprises culturing a population of cells procured, isolated and collected in accordance with any of the methodologies of the present invention and as exemplified but not limited by FIGS. 5 through 12, or other collection methodology. The population of cells are cultured in accordance with the culturing methods of the present invention. The culturing of the population of cells may occur after the placental cells are collected and suspended as illustrated in FIGS. 4 through 12. After cell culturing, this embodiment comprises selecting CD117 cells from the cell culture according to the selection methods and then cryopreserving the selected CD117 cells comprising maternal placental stem cells, for example, in accordance with the cryopreservation methodologies of the present invention or other suitable cryopreservation method.

A further embodiment of the invention illustrated in FIG. 13b generally comprises selecting CD117 cells comprising maternal placental stem cells from the population of cells procured, isolated and collected in accordance with any of the methodologies of the present invention and as exemplified but not limited by FIGS. 5 through 12, or other collection methodology. The selecting of the CD117 cells may occur after the placental cells are collected and suspended as illustrated in FIGS. 4 through 12. After selection, the embodiment comprises cryopreserving the selected CD117 cells, for example, in accordance with the cryopreservation methodologies of the present invention or other suitable cryopreservation method.

A further embodiment of the invention illustrated in FIG. 13c generally comprises selecting and culturing CD117 cells comprising maternal placental stem cells from the population of cells procured, isolated and collected in accordance with any of the methodologies of the present invention and as exemplified but not limited by FIGS. 5 through 12, or other collection methodology. The selection of the CD117 cells may occur after the placental cells are collected and suspended as illustrated in FIGS. 4 through 12. After selection, the embodiment comprises culturing CD117 cells comprising maternal placental stem cells in accordance with the culturing methods of the present invention. After cell culturing, the embodiment comprises selecting CD117 cells from the cell culture and then cryopreserving the selected CD117 cells, for example, in accordance with the cryopreservation methodologies of the present invention or other suitable cryopreservation method.

Yet another embodiment of the invention illustrated in FIG. 13d generally comprises culturing a population of cells procured, isolated and collected in accordance with any of the methodologies of the present invention and as exemplified but not limited by FIGS. 5 through 12, or other collection methodology. The culturing of the cells may occur after cryopreserving placental cells as shown in FIGS. 4 through 12 and then thawing the cryopreserved placental cells. After cell culturing, this embodiment comprises selecting CD117 cells comprising maternal placental stem cells from the cell culture and then cryopreserving the selected CD117 cells, for example, in accordance with the cryopreservation methodologies of the present invention or other suitable cryopreservation method.

Yet a further embodiment of the invention illustrated in FIG. 13e generally comprises selecting CD117 cells from the population of cells procured, isolated and collected in accordance with any of the methodologies of the present invention and as exemplified but not limited by FIGS. 5 through 12, or other collection methodology. The selecting of the CD117 cells may occur after cryopreserving placental cells as shown in FIGS. 4 through 12 and then thawing the cryopreserved placental cells. After selection, the embodiment comprises cryopreserving the CD117 placental cells comprising maternal placental stem cells, for example, in accordance with the cryopreservation methodologies of the present invention or other suitable cryopreservation method.

A further embodiment of the invention illustrated in FIG. 13f generally comprises selecting and culturing CD117 cells comprising maternal placental stem cells from the population of cells isolated and collected in accordance with any of the methodologies of the present invention and as exemplified but not limited by FIGS. 5 through 12, or other collection methodology. The selecting of the CD117 cells may occur after cryopreserving placental cells as shown in FIGS. 4 through 12 and then thawing the cryopreserved placental cells. After selection, the embodiment comprises culturing CD117 cells comprising maternal placental stem cells in accordance with the present invention. After cell culturing, the embodiment comprises selecting CD117 cells comprising maternal placental stem cells from the cell culture and then cryopreserving the CD117 cells comprising maternal placental stem cells, for example, in accordance with the cryopreservation methodologies of the present invention or other suitable cryopreservation method.

Yet a further additional embodiment of the invention illustrated in FIG. 13g generally comprises thawing a cryopreserved population of cells comprising maternal placental stem cells collected in accordance with any of the methodologies of the present invention and as exemplified but not limited by FIGS. 5 through 12, or other collection methodology. The thawing may occur after cryopreserving placental cells as shown in FIGS. 4 through 12. After thawing, the embodiment comprises culturing the cells in accordance with the invention and then selecting CD117 placental cells from the culture. The present invention comprises further culturing the selected CD117 cells comprising maternal placental stem cells, and then cryopreserving the cultured CD117 placental cells comprising maternal placental stem cells, for example, in accordance with the cryopreservation methodologies of the present invention or other suitable cryopreservation method.

The aforementioned embodiments of the present invention as referenced in FIGS. 13a through 13g are described in more detail as provided hereinafter under the following headings: CD-117 Cell Selection, CD117 Cell Separation, Preparation for Cell Culture, Cell Culture, Cell Lines, Cell Line 23a, Cell Line 23b, Preparation for Cell Culture for Cell Lines 23a and 23b, Cell Culture for Cell Line 23a, Cell Culture for Cell Line 23b, Cell Lines PLE02-PLE06, Cell Culture for Cell Line PLE02, Cell Culture for Cell Line PLE03, Cell Culture for Cell Line PLE04, Cell Culture for Cell Line PLE05, Cell Culture for Cell Line PLE06, CD117 Cell Selection for Cell Culture, and Analysis of Cell Lines.

CD-117 Cell Selection

A population of cells obtained in accordance with any of the methodologies of the present invention or other method comprises maternal placental stem cells expressing CD117. The present invention comprises the further steps of culturing the population of cells and/or selecting placental cells expressing CD117 as shown generally in FIGS. 13a through 13g and described in further detail herein.

The steps of selecting and isolating maternal placental stem cells expressing CD117 from the population of cells may occur (a) after the population of cells comprising maternal placental stem cells expressing CD117 are collected and concentrated through several centrifugation steps and cultured as disclosed herein and referenced in FIG. 13a; (b) after the population of cells comprising maternal placental stem cells expressing CD117 are collected and concentrated through several centrifugation steps as disclosed herein and referenced in FIG. 13b; (c) after the population of cells comprising the maternal placental stem cells expressing CD117 are cryopreserved and then thawed and cultured as disclosed herein and referenced in FIG. 13d; (d) after the population of cells including the maternal placental stem cells expressing CD117 are cryopreserved and then thawed as disclosed herein and referenced in FIG. 13e; and (e) at any other suitable time in the practice of the invention when CD117 cells may be selected from a population of cells, such as for example, as shown in FIGS. 13c and 13g. The steps of selecting and isolating maternal placental stem cells expressing CD117 and, optionally, any other cell expressing CD117 collected in accordance with the methods of the present invention provides a population of cells enriched for maternal placental stem cells expressing at least the cell surface marker CD117, which may be used for further cell culture or cryopreserved in accordance with the methodologies of the present invention.

The step of selecting maternal placental stem cells expressing CD117 from the population of cells comprises labeling placental cells with anti-human CD117 antibodies and then labeling the CD117 stem cell-anti-human CD117 antibody complexes with magnetically-labeled antibodies capable of binding to the anti-human CD117 antibodies. Additionally, the method comprises labeling maternal placental stem cells expressing CD117 with anti-human CD117 antibodies and then labeling the CD117 cell-anti-human CD117 antibody complexes with magnetically-labeled antibodies capable of binding to the anti-human CD117 antibodies. The method of selecting maternal placental stem cells expressing CD117 may include selecting maternal placental stem cells expressing CD117 that is collected in accordance with any of the methodologies of the present invention and as exemplified but not limited by FIGS. 5 through 12, or other collection methodology. The step of isolating maternal placental stem cells comprises exposing the complexes comprising CD117 cells, anti-human CD117 antibodies, and magnetically-labeled antibodies to a magnetic field to draw the magnetically-labeled antibodies and the rest of the complex to the column, and washing all other CD117 negative cells through the column for analysis.

Throughout the steps of selecting and isolating maternal placental stem cells expressing CD117, the population of cells and working buffer may be maintained at a cold temperature. The population of cells comprising maternal placental stem cells suspended in a wash solution if the steps of selecting and isolating placental cells expressing CD117 occurs (a) after concentration of placental cells, suspension of the pellet comprising maternal placental stem cells and culturing the cellular suspension as shown in FIG. 13a, or (b) after concentration of placental cells and suspension of the pellet comprising maternal placental stem cells as shown in FIG. 13b. Alternatively, the population of cells comprising maternal placental stem cells may be suspended in cryopreservation agent if the steps of selecting placental cells expressing CD117 occurs (a) before cryopreservation, thawing and culturing the cells as shown in FIG. 13d or 13g, or (b) after cryopreservation and thawing as shown in FIG. 13e.

The cellular suspension comprising maternal placental stem cells may be centrifuged at about 300 g for about 10 minutes at about 4° C. The pellet may be suspended in a working buffer with anti-human CD117 antibodies. The working buffer may comprise, for example and without limitation, PBS at about pH 7.2, bovine serum albumin, EDTA and about 0.09% Azide (or suitable replacement) (BD Biosciences). The pellet comprising maternal placental stem cells may be suspended, for example, in about 100 ul of working buffer and about 5 ug of purified antibodies having affinity for human CD117. The antibody may be monoclonal or polyclonal. The antibody may be purified IgG or other antibody capable of binding human CD117. The antibody may be a mouse anti-CD117 antibody. For example, the antibody may be a monoclonal mouse anti-human CD117 antibody (available as 104D2 from Santa Cruz or YB5.58 from BD Biosciences).

The solution comprising the placental cells comprising maternal placental stem cells, working buffer and anti-CD117 antibodies are incubated for an incubation period. For example, the incubation period may comprise between about 20 minutes to about 25 minutes on ice. The incubation period may, alternatively, be shortened to less than about 20 minutes if the temperature is at least about 2° C. to about 8° C. or about 5 to about 10 minutes if at least at room temperature. After the incubation period, the solution with the cells may be washed with working buffer to remove unbound antibody and then centrifuged. For example, the centrifugation may occur at about 300 g for about 10 minutes at about 4° C. After centrifugation, the supernatant is aspirated and may be saved for analysis, and the pellet is suspended in working buffer. For example, the volume of the working buffer may be about 80 ul.

A second batch of antibodies having microbeads affixed thereto and having an affinity for the anti-human CD117 antibody are added to the working buffer used to suspend the pellet. The microbeads may comprise, for example, iron oxide and polysaccharide. The microbeads may be biodegradable. Suitable microbeads are available through Miltenyi Biotec. For example, the second batch of antibodies are specific for an antibody having affinity for human CD117, such as for example, a goat anti-mouse IgG antibody. The antibody may be monoclonal or polyclonal. The antibody may be capable of binding to the light chain and/or the heavy chain of mouse antibodies. The antibody may be, for example, a goat anti-mouse IgG microbead conjugate available through Miltenyi Biotec as product 130-048-401. A 2 ml vial of the aforementioned goat anti-mouse IgG may be used for approximately 1.0×10ˆ9 of total un-separated cells.

The cellular suspension is incubated for a second incubation period. For example, the incubation period may be in a range of about 30 minutes to about 35 minutes at about 4° C. Alternatively, the incubation period may be less than about 30 minutes where the incubation occurs at about 2° C. to about 8° C. or about 5 minutes to about 10 minutes where incubation occurs at about room temperature. After the incubation period is complete, the cells are washed with working buffer, such as for example, about 2 ml of working buffer, and the cells are then centrifuged. For example, the centrifugation may occur at about 300 g for about 10 minutes at about 4° C. The supernatant may be aspirated and saved for analysis, and the pellet containing cells is suspended in working buffer, such as for example, about 500 ul of working buffer.

CD117 Cell Separation

The CD117 placental cells comprising maternal placental stem cells may be separated from a cellular suspension in working buffer using a MS column to separate the CD117 stem cells. For example, an MS Column (Miltenyi Biotec) or other suitable column may be used. Alternatively, other suitable methods to separate cells may be used such as negative depletion, other positive selections that incorporate the CD117 surface marker, aldehyde dehydrogenase separation, filtration, starch separation, centrifugation techniques including automated processing with centrifugation (i.e., Sepax, Biosafe) and serum deprivation. A MiniMACS kit available through Miltenyi Biotec comprising a unit, multistand, MS columns and microbeads may be used for CD117 cell selection. The MS column may be prepared by rinsing it with working buffer. For example, the volume of working buffer used to rinse the column may be about 500 ul. The column is placed in a magnetic field of a MACS separator available through Miltenyi Biotec or suitable separator providing a magnetic field.

The cellular suspension in working buffer is added to the column with a pipette or other device capable of transferring a volume of liquid. The CD117 cells labeled with anti-human CD117 antibodies, which are bound with antibodies attached to microbeads, are held in the column due to the magnetic field of the MACS separator. Any unlabeled cells, along with the working buffer, should flow through the column and may be collected in a sterile tube for cell phenotyping and cell count. The unlabeled cells, which flow through the column, may be identified as a negative fraction. The column may be washed with working buffer after adding the cellular suspension. For example and not as a limitation, the column may be washed at least three times or any other suitable amount of time that causes substantially all of the unlabeled cells to pass through the column. The effluent from the washing steps may be collected for cell phenotyping and count. The effluent may also be identified as a negative fraction.

The labeled CD117 stem cells are collected from the column after the column is washed. The labeled CD 117 cells are collected by placing a sterile tube under the column and removing the column from the magnetic field. Once the column is removed from the magnetic field, the labeled CD117 cells comprising maternal placental stem cells pass through the column and into the sterile tube. Residual labeled CD 117 cells comprising maternal placental stem cells in the column may be washed out by adding working buffer to the column to wash the cells through the column and, optionally, by stripping the column with a plunger to release the cells. The collected labeled CD117 cells comprising maternal placental stem cells may be identified as the positive fraction. In order to obtain a more purified population of labeled CD117 cells comprising maternal placental stem cells, the positive fraction may, optionally, be run through a column at least one more time following the previously disclosed washing procedure. The positive fraction may be centrifuged at about 300 g for about 10 minutes at about 4° C. and the supernatant aspirated. The pellet may be suspended in about 5 ml of working buffer.

The positive fraction and the negative fraction are analyzed with a hemocytometer to obtain a total count of viable cells. The negative fraction is analyzed by flow cytometry for phenotyping. Optionally, the positive fraction may also be analyzed by flow cytometry for phenotyping.

The positive fraction containing CD117 cells comprising maternal placental stem cells is prepared for cryopreservation in accordance with the methods of the present invention and described herein in further detail. About one ml of human serum albumin, about 3 ml of DPBS and about one ml of DMSO are added to the about 5 ml of the positive fraction. Alternatively, other tissue culture media may be used in the step of preparing the CD117 cells for cryopreservation, such as for example, complete media, bovine serum albumin, fetal calf serum or protein plasma fraction. The solution containing CD117 cells is mixed and cooled on ice for about 10 minutes. About one ml of DMSO is added as a cryopreservative. Alternatively, about one ml of a mixture of about 6% HES hydroxyethyl starch and about 5% DMSO may be used as a cryopreservative. The resulting solution is aliquoted into cryovials. Alternatively, the resulting solution may be aliquoted into any container suitable for cryopreservation, such as for example, a cryopreservation bag. The cryovials or other suitable container are then cryopreserved in a controlled rate freezer (Cryomed) in accordance with controlled rate freezer protocol of the present invention as described herein in further detail. Once the solution containing CD117 cells comprising maternal placental stem cells reaches the target temperature of about −90° C., the cryovials or other suitable container are transferred into a long term storage freezer and stored at about −135° C. or less. Alternatively, the cryovials or other suitable cryopreservation container may be placed into a monitored dump freeze and frozen to about −80° C. and then transferred into the vapor phase of liquid nitrogen in a long term storage freezer at about −135° C. or less.

Alternatively, the positive fraction may be used to seed culture flasks and culture the cells in accordance with the methods of the present invention. The CD117 cells comprising maternal placental stem cells may then be selected from the cell cultures and cryopreserved in accordance with the methods of the present invention as shown, for example, in FIGS. 13c and 13g. Alternatively the cells may be concentrated for CD117 by other suitable methods to separate cells such as negative depletion, other positive selections that incorporate the CD117 surface marker, aldehyde dehydrogenase separation, filtration, starch separation, centrifugation techniques including automated processing with centrifugation (ie Sepax, Biosafe) and serum deprivation.

Preparation for Cell Culture

The population of cells comprising maternal placental stem cells collected in accordance with the methodologies of the present invention may be cultured to further select CD117 cells from the cell culture. The population of cells may be prepared for cell culture after concentration according to the present invention or after being cryopreserved and thawed.

The thawing method comprises preparing aliquots of about 15 ml of density gradient media available as Histopaque through Sigma-Aldrich or other suitable media at about room temperature for each vial containing about 5 ml of cryopreserved cells to be thawed; and then preparing about 25 ml aliquots of Chang's complete media, DMEM complete media or other suitable media for each vial containing about 5 ml of cryopreserved cells to be thawed.

Chang's complete media comprises about 325 ml of MEM alpha media available through Gibco as product 12571-063, about 90 ml of Chang B (basal) C110 (18% v/v) available through Irvine Scientific, about 10 ml of Chang medium C from Supplement C106 (2% v/v) available through Irvine Scientific, about 5 ml Penicillin/Streptomycin (liquid prepared with 10,000 units/ml Penicillin G Sodium and 10,000 ug/ml Streptomycin sulfate in 0.85% saline available through Gibco as product 15140-122, about 5 ml of L-glutamine 200 mM (100×) available through Gibco as product 25030-081, and about 75 ml of ES-Fetal Bovine Serum (15% v/v) available through Gibco as product 10439-024.

If thawing of a cryopreserved sample is necessary, the cryopreserved cells comprising maternal placental stem cells are thawed by removing the vials from the vapor phase of the liquid nitrogen storage freezer. The vials are placed in about a 37° C. to 40° C. water bath and agitated. The cells comprising maternal placental stem cells should not be allowed to completely thaw, but the vials should contain ice. The thawed cells should be diluted by placing the about 5 ml aliquot into the about 25 ml aliquot of chilled Chang's complete media containing about one mg of DNase available through Pulmozyme.

Alternatively, and if thawing is not necessary, such as for example, when the population of cells comprising maternal placental stem cells is cultured in the absence of the step of cryopreservation, the cellular suspension comprising maternal placental stem cells should be diluted by placing the about 5 ml aliquot into the about 25 ml aliquot of chilled Chang's complete media containing about one mg of DNAse available through Pulmozyme.

The diluted cell suspension may be mixed by inversion. The suspension comprising maternal placental stem cells is centrifuged at about 840 g for about 7 minutes at about 20° C. The supernatant is aspirated while not disturbing the pellet. The pellet is brought up to a total volume of about 30 ml Chang's complete media. A small amount of the cellular suspension comprising maternal placental stem cells is removed for analysis that comprises cell count with a hemocytometer and viability testing using trypan blue or other suitable viability testing methodology. The about 30 ml suspension comprising Chang's complete media and maternal placental stem cells is overlaid on a density gradient solution available as Histopaque through Sigma-Aldrich or other suitable media, and is centrifuged without a brake at about 420 g for about 30 minutes at about 20° C. The tube is removed from the centrifuge without disrupting the buffy coat. The supernatant is aspirated and the buffy coat comprising maternal placental stem cells is collected. The buffy coat is brought up to about 20 ml with Chang's complete media and is washed at about 840 g for about 7 minutes. The supernatant is aspirated, and the pellet is suspended in Chang's complete media up to about 10 ml but may also up to about 20 ml or about 30 ml, or even less than about 10 ml. An aliquot of the suspension, such as for example, about 100 ul is removed to perform a cell count and viability analysis.

Cell Culture

The cells in suspension may be seeded at about 40,000 cells/cm² into an untreated tissue culture flask in Chang's complete media, DMEM complete media (with high glucose or low glucose), or other suitable media. The flask should be incubated in about 5% CO₂ in a CO₂ incubator available through Thermo Electron Corp. or Bioscience Technologies, or any other suitable incubator system at a temperature of about 37° C. The cell cultures are monitored for turbidity and pH change. If the pH is high, about 50% of the media should be changed.

The flask may be incubated initially for about 7 days or until the media is significantly out of range as indicated by the color of the phenol red indicator in the media. If the pH remains stable after about 7 days, the media may be changed with fresh media (also referred to herein as “virgin media”) as necessary. Only about half of the media may be changed to maintain continuous factors that have been excreted into the media by the cells. After the media change at day 7, the cells may become confluent by day 8 to day 21. Once attaining about 70-80% confluence, the cells may be sub-cultured. Cells are sub-cultured using a trypsin-like enzyme such as TrypLE™ Express available through Gibco, or any other suitable enzyme to provide enough cells to perform the CD117 cell selection in accordance with the present invention. For example, cell selection may occur with about 10 million cells. CD117 cell selection may also occur with greater than or less than about 10 million cells.

In accordance with the invention, CD117 cells may be collected from the cell culture at a suitable time. In order to collect the CD117 cells, adherent cells may be dissociated from the flask. In order to dissociate the cells from the flask, the media is aspirated via an automated pipette. The flask is then rinsed with about 5 ml of Phosphate Buffered Saline (PBS) without calcium or magnesium. The PBS is then removed from the flask with attached cells that have been washed at least once. About one ml of a Trypsin-like recombinant enzyme such as TrypLE™ Express available through Gibco, or any other suitable enzyme, should be added, preferably pre-warmed at about 37° C., to the cell culture in the flask. The flask is agitated to coat the cells with the enzyme. The flask with enzyme should be incubated for about 5 minutes at about 37° C. After incubation, the flask should be gently tapped on a solid surface to dislodge the cells. The flask may be diluted with about 2 ml of Chang's complete media, and the cells transferred to a 15 ml centrifuge tube for washing with Chang's complete media, DMEM complete media (with high glucose or low glucose), or other suitable media. The tube may be centrifuged for about 7 minutes at about 100 g at about 20° C. The supernatant is aspirated and discarded. The pellet is suspended in a suitable volume of Chang's complete media, DMEM complete media (with high glucose or low glucose), or other suitable media.

At this point, the CD117 cells may be selected from the cell culture in accordance with CD117 cell selection methodologies of the present invention. Once selected, the CD117 cells may be plated on a Petri dish, seeded into a tissue culture flask or cryopreserved in accordance with present invention.

The cells may be plated in a 9 cm² Petri dish using Chang's complete media (about 15% FBS). Alternatively, the cells may be placed in a tissue culture flask with a vented cap. If the pH of the media becomes high, the cells may be washed with Chang's complete media. When necessary after suitable growth, the cells may be dissociated from the Petri dish or tissue culture flask using a trypsin-like enzyme and then placed in an untreated tissue culture flask using Chang's complete media. After suitable growth, the cells may be dissociated using a trypsin-like enzyme such as TrypLE™ Express available through Gibco and then seeded in a fresh untreated tissue culture flask. This process may be repeated in order to maintain desired cell growth. The cells may be washed with fresh media, or about 50% of the media or other suitable amount may be replaced with fresh media if the pH of the media is high. At this point, the CD117 cells comprising maternal placental stem cells may be selected from the cell culture in accordance with the CD 117 cell selection methodology of the present invention. The selected CD 117 cells may be plated on a Petri dish, seeded into a tissue culture flask or cryopreserved in accordance with present invention.

Cell Lines

Several placental cell lines have been developed from the practice of the methodologies of the invention. Cell Lines 23a and 23b were obtained from the same placenta.

Cell Line 23a

A human placenta was procured and placental tissue was obtained with scalpel and forceps in accordance with the methodologies of the present invention generally shown in FIG. 11 and as described in further detail herein. After delivery and as measured on the fetal side of the placenta, an about 4 cm by about 4 cm piece of placental tissue cut through the depth of the placenta from fetal side to placental side was procured. The piece of placental tissue was about 50% thickness of the placenta and comprised about 50% fetal tissue and about 50% maternal tissue. The piece of placental tissue was labeled, transported, and processed at the processing facility in accordance with the packaging and transportation methods of the present invention (i.e., packaged in a contained to maintain the biopsies at a temperature between about 1° C. to about 10° C. for the duration of shipment, and shipped to and received at a processing facility within about 24 hours of procurement).

At the processing facility and in a clean room, the piece of placental tissue was subjected to microbiological quality control at pre-processing of the sample, and portions of the maternal placental tissue were removed from the fetal placental tissue, which was disinfected (i.e., double treatment with Betadine and antibiotic dip solutions and washed), all in accordance with methods of the present invention. The disinfected piece of placental tissue was about 50% of the overall thickness of the whole placenta. The disinfected piece of placental tissue comprised about 50% fetal tissue and about 50% maternal tissue.

The disinfected placental tissue was disaggregated by mechanical separation into pieces of minced tissue. The placental cells comprising maternal placental stem cells were collected by straining the minced tissue against a cell strainer and washing the minced tissue with wash solution. The placental cells comprising maternal placental stem cells present in wash solution in a tube were centrifuged at about 2000 RPM for about 7 minutes at about 4° C. The placental cells comprising maternal placental stem cells were re-suspended in wash solution and prepared for cell culture as discussed hereinafter.

Cell Line 23b

Placental tissue from the aforementioned placenta was procured with scalpel and forceps in accordance with the methodologies of the invention generally shown in FIG. 9 and as described in further detail herein. After delivery and as measured on the fetal side of the placenta, an about 4 cm by about 4 cm piece of placental tissue cut through the depth of the placenta from fetal side to placental side was procured. The piece of placental tissue was about 50% thickness of the placenta and comprised about 50% fetal tissue and about 50% maternal tissue. The piece of placental tissue was labeled, transported, and processed at the processing facility in accordance with the packaging and transportation methods of the present invention (i.e., packaged in a contained to maintain the biopsies at a temperature between about 1° C. to about 10° C. for the duration of shipment and shipped to and received at a processing facility within about 24 hours of procurement).

At the processing facility and in a clean room, the piece of placental tissue was subjected to microbiological quality control at pre-processing of sample, and portions of the maternal placental tissue were removed from the fetal placental tissue, which was disinfected (i.e., double treatment with Betadine and antibiotic dip solutions and washed), all in accordance with methods of the present invention. The disinfected piece of placental tissue was about 50% of the thickness of the whole placenta. The piece of placental tissue comprised about 50% fetal tissue to about 50% maternal tissue.

The disinfected placental tissue was minced and disaggregated by enzymatic digestion (i.e., about 10 g of placental tissue suspended in about 24 ml of collegenase solution (50 mg/ml) for 45 minutes at about 37° C. The digestions was stopped using about 10 ml of HSA (25%) in each tube of enzyme solution and digested tissue. The tubes were inverted to mix and the tissue was removed. Placental cells comprising maternal placental stem cells were collected and centrifuged at about 2000 RPM for about 7 minutes at about 4° C. The pelleted cells comprising maternal placental stem cells were re-suspended in wash solution to about 45 ml. The placental cells comprising maternal placental stem cells were concentrated by centrifugation at about 2000 RPM for about 7 minutes at about 4° C. The pellets comprising maternal placental stem cells were re-suspended in wash solution and prepared for cell culture as described hereinafter.

Preparation for Cell Culture of Cell Lines 23a and 23b

The populations of cells comprising maternal placental stem cells relating to cell lines 23a and 23b were cultured separately from one another according to the culture methods and processes of the present invention.

The cellular suspensions of the populations of cells comprising maternal placental stem cells relating to cell lines 23a and 23b were separately and independently prepared for cell culture according to the following methods. Each cellular suspension was overlaid on 15 ml of a density gradient solution with 1.083 g/ml (Histopaque, Sigma-Aldrich). Each suspension and density gradient solution was centrifuged without a brake at about 420 g for about 30 minutes at about 20° C. Each tube was removed from the centrifuge without disturbing the buffy coat. The supernatant was aspirated from each tube, and each buffy coat was removed and placed in separately labeled tubes so that the population of cells relating to cell lines 23a and 23b were separate from one another. DMEM complete media was added to the buffy coat layer to bring the total volumes of each separate buffy coat up to about 30 ml. The solution was washed twice by centrifugation at about 840 g for about 7 minutes at about 4° C. After centrifugation on the second wash, each supernatant was removed from each tube leaving a pellet comprising maternal placental stem cells. About 15 ml of DMEM complete media was added to each tube to suspend each pellet. About 100 uL of suspension was removed from each tube to perform separate cell count analysis with a hemocytometer and viability analysis using trypan blue.

Cell Culture for Cell Line 23a

About one million cells comprising maternal placental stem cells obtained from the population of cells suspended in DMEM complete media were seeded at about 40,000 cells/cm² into a T25 untreated tissue culture flask in DMEM high glucose media on day 1. The cells are associated with those cells collected from the piece of placenta associated with Cell Line 23a. Flasks were incubated in about 5% CO₂ at about 37° C. temperature. On day 3, the cells went through passage 1 and the media was switched to 15% Chang's complete media. Chang's complete media was used for the duration of the cell culture.

Each passage generally involved dissociating cells from the flask by aspirating the media with an automated pipette. The flask was rinsed with about 5 ml of Phosphate Buffered Saline (PBS) without calcium or magnesium. The PBS was then removed after the flask with cells attached had been washed once. About one ml of Trypsin-like enzyme such as TrypLE™ Express available through Gibco was added, preferably pre-warmed at about 37° C., to the flask, and the flask was agitated to coat the cells with the enzyme. The flask with the enzyme was incubated for about 5 minutes at about 37° C. in an incubator. After incubation, the flask was gently tapped on a solid surface to dislodge the cells. The contents of the flask were diluted with about 2 ml of complete media, and the cells were transferred to a 15 ml centrifuge tube for washing. The tube was centrifuged for about 7 minutes at about 100 g at about 20° C. The supernatant was discarded after centrifugation, and the harvested cells were suspended in Chang's complete media and then placed into at least one new T25 or T75 untreated cell culture flask.

Additional passages occurred on days 6, 9, and 12. On day 12, 440,000 cells were harvested and cryopreserved. The harvested cells are identified as Cell Line 23a.

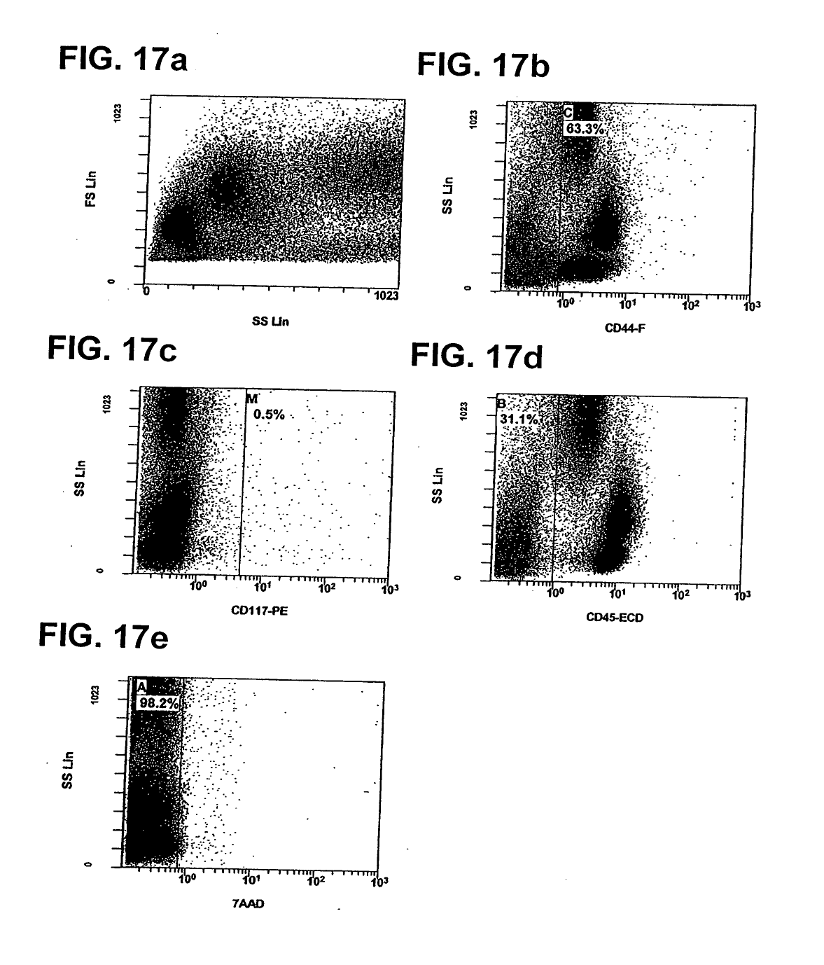

Phenotyping and validity assessment was performed in accordance with the methods described herein for all of the cell culture experiments relating to Cell Line 23a as shown in FIGS. 17a through 17e. Cell counts during cell culture were performed using a hemocytometer relating to all of the cell lines. As summarized in Table 7, the data collected from the assessment showed that the cells of Cell Line 23a expressed CD44 and CD117 and had low expression of CD45 with a high percentage of viability at Passage 0 of the cell culture.

TABLE 7
Phenotype and Validity Analysis of Cell Line 23a

| | | | CD45- | |
|---|---|---|---|---|
| | CD117- | CD44- | ECD | 7AAD- |
| Passage # | PE | FITC | (NEG) | TEST |
| FICOL | 0.5% | 25.8% | 66.9% | 98.2% |
| P0 | 12.9% | 16.7% | 88.1% | 91.5% |

Cell Culture for Cell Line 23b

About one million cells comprising maternal placental stem cells obtained from the population of cells suspended in DMEM complete media were seeded at about 40,000 cells/cm² into a T25 untreated tissue culture flask in DMEM high glucose media on day 1. The cells are associated with those cells collected from the piece of placenta associated with Cell Line 23b. Flasks were incubated in about 5% CO₂ at 37° C. temperature. At day 42 and at passage 7, 15% Chang's complete media was used as culture media. For the duration of the cell culture, 15% Chang's complete media was used. Thirty-nine additional passages occurred over 112 days of cell culture of Cell Line 23b. Aliquots of cells and media were collected throughout the cell culture for phenotyping and validity assessment.

Phenotyping and validity assessment was performed in accordance with the methods of the present invention and described hereinafter for all of the cell culture experiments relating to all of the cell lines. Cell counts during cell culture were performed using a hemocytometer relating to all of the cell lines. As summarized on Table 8, the data collected from the assessment showed that the cells of Cell Line 23b expressed CD44, CD117 and CD166 and had low expression of CD45 with a high percentage of viability at various passages throughout the duration of the cell culture.

TABLE 8
Phenotype and Validity Analysis of Cell Line 23b

| | | | | CD45- | | | | | |
|---|---|---|---|---|---|---|---|---|---|
| | CD117- | CD166- | CD44- | ECD | 7AAD- | CD105- | CD29- | CD34- | CD90- |
| Passage # | PE | PE | FITC | (NEG) | TEST | PE | FITC | ECD | PC5 |
| FICOL | 0.4% | N/A | 63.0% | 31.1% | 98.2% | N/A | N/A | N/A | N/A |
| P2 | 15.5% | N/A | 98.4% | 95.8% | 99.7% | N/A | N/A | N/A | N/A |
| P4 | N/A | 97.9% | N/A | 94.9% | 99.8% | 97.0% | 95.3% | 99.7% | N/A |
| P7 | N/A | 97.8% | N/A | 87.1% | 99.8% | N/A | N/A | N/A | N/A |
| P11 | 13.0% | 96.9% | 93.1% | 98.1% | 99.6% | 95.3% | 93.7% | 0.2% | 52.8% |
| P14 | 2.8% | 95.5% | 96.8% | 94.6% | 99.5% | 97.4% | 97.1% | 0.4% | 56.4% |
| P20 | 5.2% | 94.9% | 92.1% | 99.2% | 98.2% | 97.8% | 98.1% | 0.9% | 96.5% |
| P21 | 6.1% | 92.5% | 94.2% | 95.9% | 99.3% | 90.7% | 89.4% | 0.7% | 96.6% |
| P24 | 3.3% | 89.9% | 91.2% | 89.4% | 99.2% | 87.9% | 86.1% | 0% | 95.2% |
| P26 | 1.9% | 86.5% | 88.6% | 88.5% | 97.4% | 82.9% | 80.6% | 0% | 97.6% |
| P30 | 1.3% | 74.5% | 69.5% | 76.6% | 97.8% | 58.3% | 53.0% | 0% | 91.3% |
| P38 | 4.3% | N/A | 97.5% | 98.9% | 99.8% | N/A | N/A | N/A | N/A |
| P39 | 2.8% | N/A | 97.3% | 99.7% | 99.9% | N/A | N/A | N/A | N/A |

Cell Lines PLE02-PLE06

Cell Lines PLE02, PLE03, PLE04, PLE05 and PLE06 were obtained after processing placental tissue obtained from different placentas in accordance with the methodologies of the present invention and described in further detail herein. Each placenta was associated with a male child.

Five different human placentae were delivered and a piece of placental tissue was procured from each placenta and processed independently for each placenta with scalpel and forceps in accordance with the methodologies of the invention generally shown in FIG. 9 and as described in further detail herein. As measured on the fetal side of the placenta, an about 8 cm by about 8 cm piece of placental tissue cut through the depth of the placenta from fetal side to placental side was obtained. Each piece of placental tissue was about 50% thickness of the placenta and comprised about 50% fetal tissue and about 50% maternal tissue. Each piece of placental tissue procured from each placenta was separately and individually labeled, packaged and transported to the processing facility in accordance with the packaging and transportation methods of the present invention (i.e., packaged in a contained to maintain the biopsies at a temperature between about 1° C. to about 10° C., shipped and then received at a processing facility within about 48 hours of procurement).

Each piece of placental tissue was received and processed in the processing facility separately from each other piece of placental tissue. Each sterile container containing DPBS and each separate piece of placental tissue was disinfected, transferred into a clean room, and placed on ice in an ice pan. Each sterile container was placed in a BSC and the top of the sterile container was removed. Samples of the DPBS buffer (about 4 ml) were removed from the sterile container using a sterile syringe and used to inoculate five BacT/ALERT blood culture bottles, one bottle for each piece of placental tissue. The BacT/ALERT blood cultures were used to identify the presence of bacterial or fungal contamination. The blood culture bottles were incubated at about 37° C. in a BacT/ALERT system for about 7 days, whereby the results indicate a positive identification for Streptococcus viridans, Escherichia fergufonii and Escherichia coli for the DPBS buffers containing placental tissue corresponding with PLE03, PLE04 and PLE05, respectively, as shown in Table 9.

TABLE 9
BacT/ALERT System Analysis

| Cell Line Associated with Each | Status for Bacterial and | | | |
|---|---|---|---|---|
| Piece of Placental Tissue | Fungal Organisms | Type | Loaded | Cell |
| PLE02 | NEGATIVE | BTA PF | 7/18/06 @ 15:44 | 4C01 |
| PLE03 | POSITIVE - Streptococcus | BTA PF | 7/20/06 @ 15:05 | 4C03 |
| | viridans | | | |
| PLE04 | POSITIVE - Escherichia | BTA PF | 7/21/06 @ 17:51 | 2A02 |
| | fergufonii | | | |
| PLE05 | POSITIVE - Escherichia coli | BTA PF | 7/21/06 @ 17:51 | 2A04 |
| PLE06 | NEGATIVE | BTA PF | 7/25/06 @ 12:06 | 2A04 |

Each piece of placental tissue was removed from the sterile container and subjected to microbiological quality control at pre-processing of sample, and portions of the maternal placental tissue were removed from the fetal placental tissue, which was disinfected (i.e., double treatment with Betadine and antibiotic dip solutions and washed), all in accordance with methods of the present invention. The disinfected piece of placental tissue was about 50% of the thickness of the whole placenta. The piece of placental tissue comprised about 50% fetal tissue to about 50% maternal tissue.

Each disinfected piece of placental tissue was separately minced and disaggregated by enzymatic digestion (i.e., about 10 g of placental tissue suspended in about 25 ml of collegenase solution (50 mg/ml) for 45 minutes at about 37° C.). Digestion was stopped using about 10 ml of HSA (25%) in each tube of enzyme solution and digested tissue. The tubes were inverted to mix and the tissue was removed Placental cells from each piece of placental tissue were separately and independently collected and centrifuged at about 2000 RPM for about 7 minutes at about 4° C. in accordance with the invention. The pelleted cells were re-suspended in wash solution to about a 45 ml volume. The placental cells were concentrated by centrifugation at about 2000 RPM for about 7 minutes at about 4° C.

After centrifugation, about one ml of the supernatant near the top of each collection tube was removed and used to inoculate a BacT/ALERT blood culture bottle. The blood culture bottle was incubated at about 37° C. in a BacT/ALERT system for about 7 days, whereby the results were negative for bacterial and fungal contamination as shown in Table 10.

TABLE 10
BacT/ALERT System Analysis

| Cell Line | | | | |
|---|---|---|---|---|
| Associated with | Status for | | | |
| Each Piece | Bacterial | | | |
| of | and Fungal | | | |
| Placental Tissue | Organisms | Type | Loaded | Cell |
| PLE02 | NEGATIVE | BTA PF | 7/18/06 @ 15:44 | 4C02 |
| PLE03 | NEGATIVE | BTA PF | 7/20/06 @ 15:05 | 4C04 |
| PLE04 | NEGATIVE | BTA PF | 7/21/06 @ 17:51 | 2A01 |
| PLE05 | NEGATIVE | BTA PF | 7/21/06 @ 17:51 | 2A03 |
| PLE06 | NEGATIVE | BTA PF | 7/25/06 @ 12:06 | 2A05 |

The remaining supernatant was aspirated while carefully leaving the pellet comprising maternal placental stem cells expressing the cell surface marker CD117. The pellet was suspended in wash solution up to about 6 ml. About one ml of cellular suspension was collected as a post-processing sample and placed in an analysis tube for flow cytometry to obtain cell count, flow cytometry analysis and cell viability. About 5 ml of the cellular suspension was further processed in preparation for cryopreservation.

The pellets comprising maternal placental stem cells were re-suspended in wash solution up to about 6 ml. The cellular suspension was prepared for cryopreservation by combining the placental cells comprising maternal placental stem cells with about 18 ml to about 20 ml of cryopreservation agent per about 18 ml to about 20 ml of placental cell suspension according to the cryopreservation preparation methods of the present invention. The populations of placental cells in cryopreservation agent were cryopreserved below about −135° C. in accordance with the cryopreservation methods of the present invention.

Each of the cryopreserved populations of cells relating to cell lines PLE02, PLE03, PLE04, PLE05 and PLE06 were thawed in preparation for cell culture independently from one another in accordance with the invention and described in further detail as follows.

The cryopreserved populations of cells relating to cell lines PLE02, PLE03, PLE04, PLE05 and PLE06 were removed from cryopreservation and thawed independently from one another and separately prepared for cell culture distinct from one another. The cryovials with cryopreserved placental cells comprising maternal placental stem cells were removed from the vapor phase of a liquid nitrogen storage freezer. The cryovials were placed in a water bath of about 37° C. and agitated. The cells did not completely thaw and contained ice.

The cells relating to cell lines PLE02, PLE03, PLE04, PLE05 and PLE06, separately from one another, were diluted by mixing with about 25 ml of chilled Chang's complete media containing about one mg of DNAse such as Pulmozyme. Each solution was mixed by inversion and centrifuged at about 840 g for about 7 minutes. Each supernatant was removed leaving the pellet comprising fetal placental stem cells. Chang's complete media was added to each pellet to bring the total volume up to about 30 ml. About 100 ul of the suspension was removed for analysis. A cell count was performed using a hemocytometer and a viability test was performed using trypan blue.

Each suspension comprising maternal placental stem cells was separately overlaid on a 15 ml density gradient solution with 1.083 g/ml (Histopaque, Sigma-Aldrich). Each suspension and density gradient solution was centrifuged without a brake at about 420 g for about 30 minutes at about 20° C. Each tube was removed from the centrifuge without disturbing the buffy coat. The supernatant in each tube was aspirated, and each buffy coat was removed and separately placed in a tube so that the population of cells relating to cell lines PLE02, PLE03, PLE04, PLE05, and PLE06 were processed separately from one another. Chang's complete media was added to the buffy coat in each tube to bring the total volume up to about 30 ml. The cellular suspension in each tube was washed twice at about 840 g for about 7 minutes at about 4° C. After centrifugation on the second wash, the supernatant in each tube was removed leaving a pellet and about 15 ml of Chang's media was added to each tube to re-suspend each pellet. About 100 uL of suspension was removed from each tube to perform discreet cell count analysis with a hemocytometer and viability analysis using trypan blue.

Cell Culture for Cell Line PLE02

About 930,000 cells comprising maternal placental stem cells obtained from the population of cells suspended in Chang's media were seeded at about 40,000 cells/cm² into a T25 untreated tissue culture flask in 15% Chang's complete media. The cells are associated with those cells collected from the piece of placenta associated with Cell Line PLE02. Fresh 15% Chang's complete media was used through the several passages of the cell culture. Flasks were incubated in about 5% CO₂ at about 37° C. temperature.

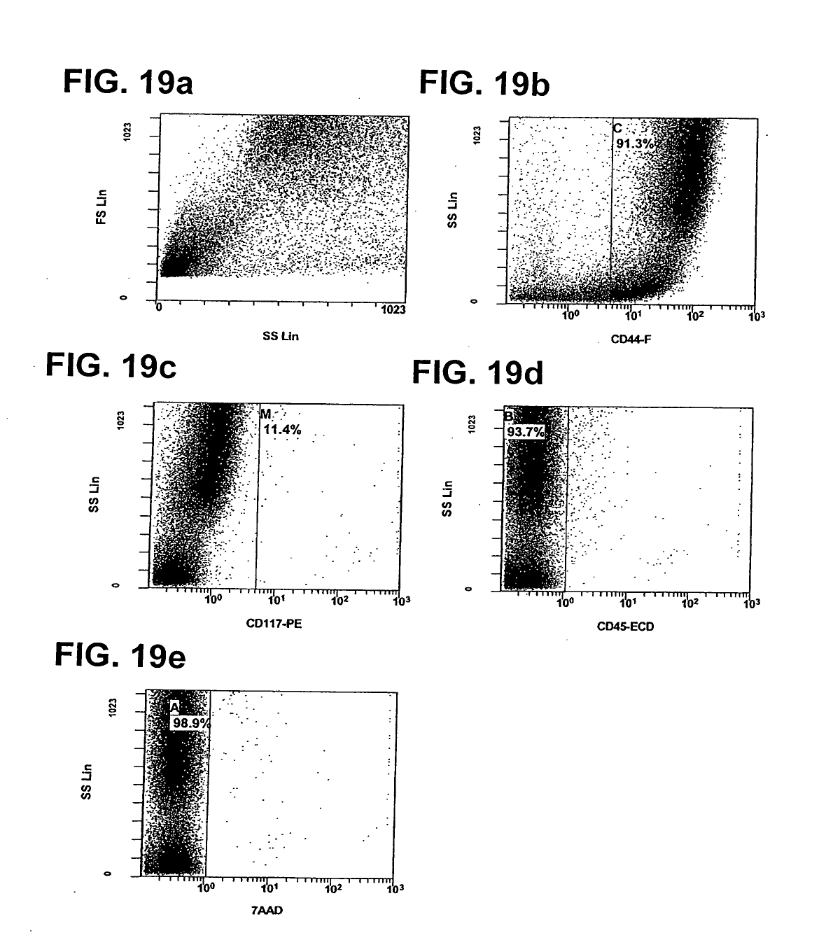

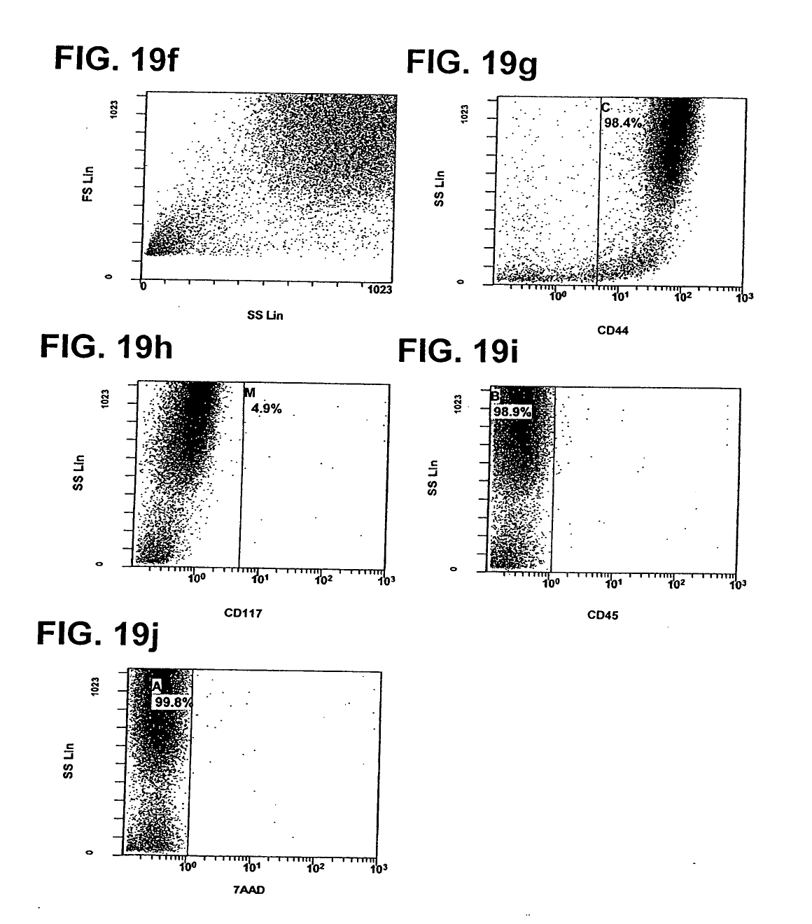

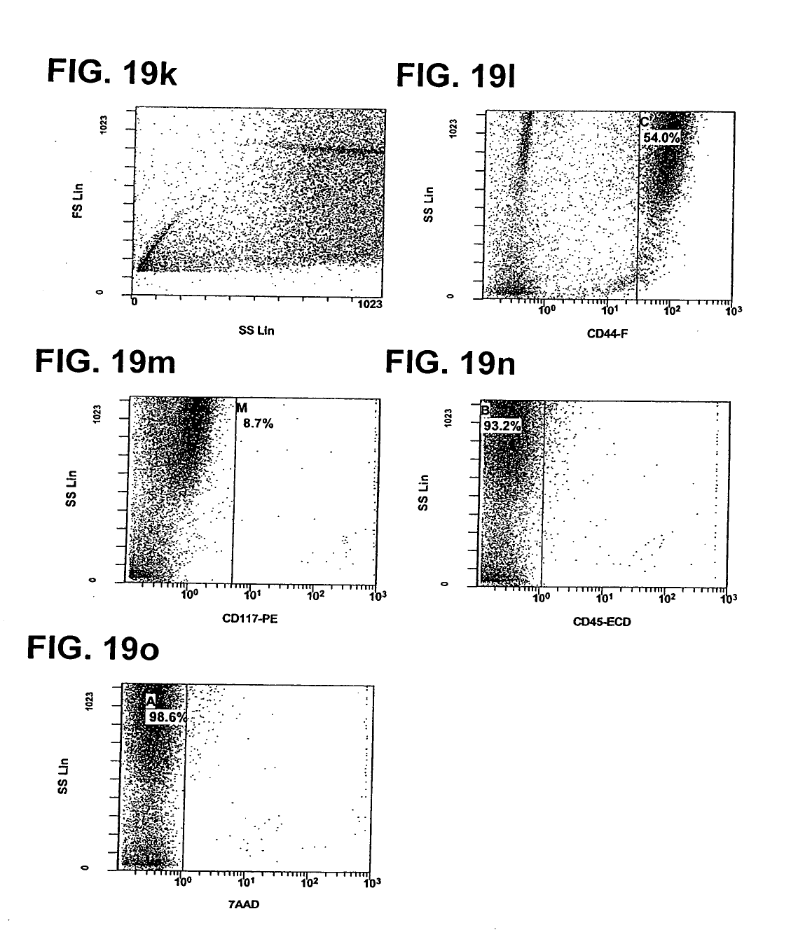

At passage 4, the harvested cells were split into three separate cell cultures in T75 untreated tissue culture flasks. At passage 5, harvested cells were split into six separate cell cultures in T75 untreated tissue culture flasks. At Passage 6, harvested cells were split into 30 untreated tissue culture flasks. At passage 7, harvested cells were selected for CD117 in accordance with the methodologies of the present invention and as described hereinafter using an MS Column (Miltenyi Biotec). Aliquots from the cell culture at days 37 and 42 and the positive fraction from the CD117 cell selection were analyzed with a hemocytometer to obtain the total number of viable cells and for cell phenotype. The cells at days 37 and 42 and in the positive fraction expressed CD44 and CD117 and had low or no expression of CD45 with a high percentage of viability as shown in FIGS. 19a through 19o.

Phenotyping and validity assessment was performed in accordance with the methods described herein for the cells of Cell Line PLE02. Cell counts during cell culture were performed using a hemocytometer relating to all of the cell lines. As summarized on Table 11, the data collected from the assessment showed that the cells of Cell Line PLE02 express CD44 and CD117 and had low or no expression of CD45 with a high percentage of viability at various passages throughout the duration of the cell culture.

TABLE 11
Phenotype and Validity Analysis of Cell Line PLE02

| | CD117- | CD44-FITC | CD45-ECD | |
|---|---|---|---|---|
| Passage # | PE | (POS) | (NEG) | 7AAD-TEST |
| PLE02C - P5 | 11.10% | 91.10% | 93.70% | 98.90% |
| PLE02C - P7 | 4.80% | 98.30% | 98.90% | 99.80% |
| PLE02C - P0 POS | 8.60% | 52.95% | 93.20% | 98.60% |
| FRAC | | | | |

Cell Culture for Cell Line PLE03

About 750,000 cells comprising maternal placental stem cells obtained from the population of cells for Cell Line PLE03 suspended in Chang's media were seeded at about 30,000 cells/cm² into a T25 untreated tissue culture flask in 15% Chang's complete media. The cells are associated with those cells collected from the piece of placenta associated with Cell Line PLE03. Fresh 15% Chang's complete media was used through the several passages the cell culture. Flasks were incubated in about 5% CO₂ at about 37° C. temperature.

At passage 1, the harvested cells were placed into a T75 untreated tissue culture flask. At passages 2, 3, 4, 5, 6 and 7, harvested cells were split into separate cell cultures in T75 untreated tissue culture flasks. At Passage 7, harvested cells were selected for CD117 in accordance with the methodologies of the present invention and as described hereinafter using an MS Column (Miltenyi Biotec). The positive fraction from the CD117 cell selection were analyzed with a hemocytometer to obtain the total number of viable cells and for cell phenotype. The cells in the positive fraction expressed CD44 and CD117 and had low or no expression of CD 45 with a high percentage of viability. The cells in the positive fraction were cultured further for 12 days in Chang's complete media in untreated tissue culture flasks. The cells in the negative fraction were cultured for 8 days in Chang's complete media in untreated tissue culture flasks.

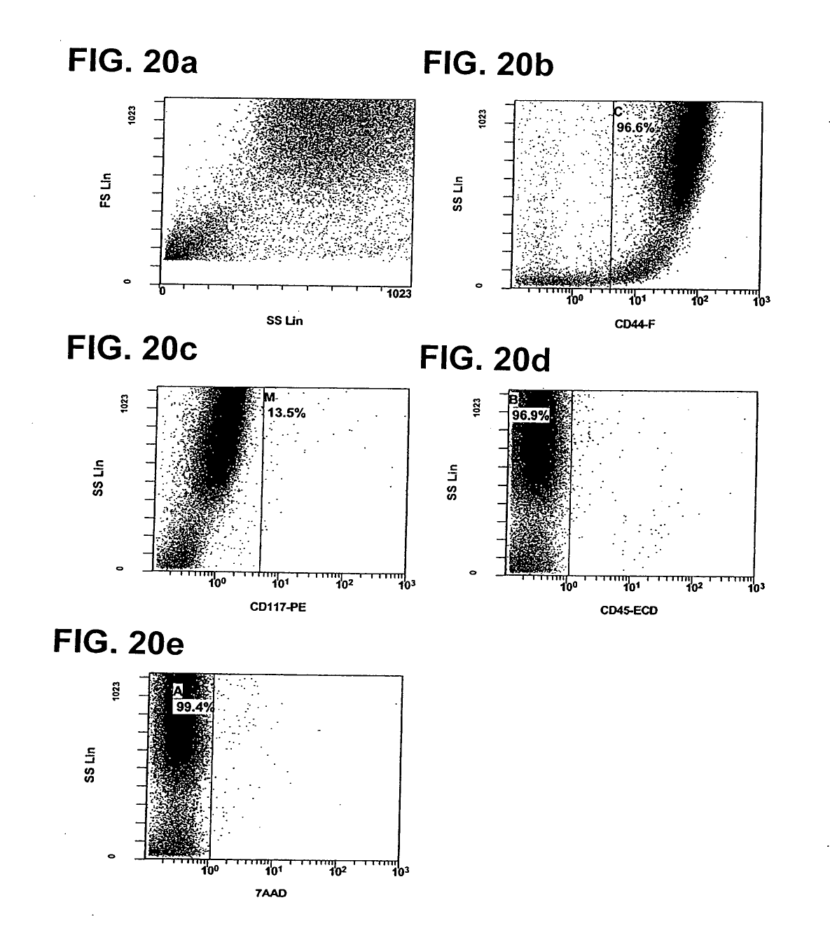

Phenotyping and validity assessment was performed in accordance with the methods described herein for the cells of Cell Line PLE03. Cell counts during cell culture were performed using a hemocytometer. As summarized on Table 12, the data collected from the assessment showed that the cells of Cell Line PLE03 express CD44 and CD117 and had low or no expression of CD45 with a high percentage of viability at various passages throughout the duration of the cell culture. The results of the phenotyping and validity assessment are shown in FIGS. 20a through 20e.

TABLE 12
Phenotype and Validity Analysis of Cell Line PLE03

| | | CD44- | CD45- | | |
|---|---|---|---|---|---|
| | CD117- | FITC | ECD | 7AAD- | 7AAD- |
| Passage # | PE | (POS) | (NEG) | TEST | ISO |
| PLE03B - P7 | 13.40% | 96.50% | 96.90% | 99.40% | 99.60% |

Cell Culture for Cell Line PLE04

About 1,000,000 cells comprising maternal placental stem cells obtained from the population of cells suspended in Chang's media were seeded at about 14,800 cells/cm² into a T25 untreated tissue culture flask in 15% Chang's complete media. The cells are associated with those cells collected from the piece of placenta associated with Cell Line PLE04. Fresh 15% Chang's complete media was used through the several passages the cell culture. Flasks were incubated in about 5% CO₂ at about 37° C. temperature.

At passage 1, the harvested cells were split into two T75 untreated tissue culture flasks. At passages 2 through 7, harvested cells were split into separate cell cultures in T75 untreated tissue culture flasks. At Passage 8, harvested cells were selected for CD117 in accordance with the methodologies of the present invention and as described herein using an MS Column (Miltenyi Biotec). The positive fraction from the CD117 cell selection were analyzed with a hemocytometer to obtain the total number of viable cells and for cell phenotype. The cells in the positive fraction expressed CD44 and CD117 and had low or no expression of CD45 with a high percentage of viability at several passages in culture. The cells in the positive fraction comprising maternal placental stem cells were cultured further for 15 days in Chang's complete media in untreated tissue culture flasks. The cells in the negative fraction were cultured for 1 day in Chang's complete media in untreated tissue culture flasks.

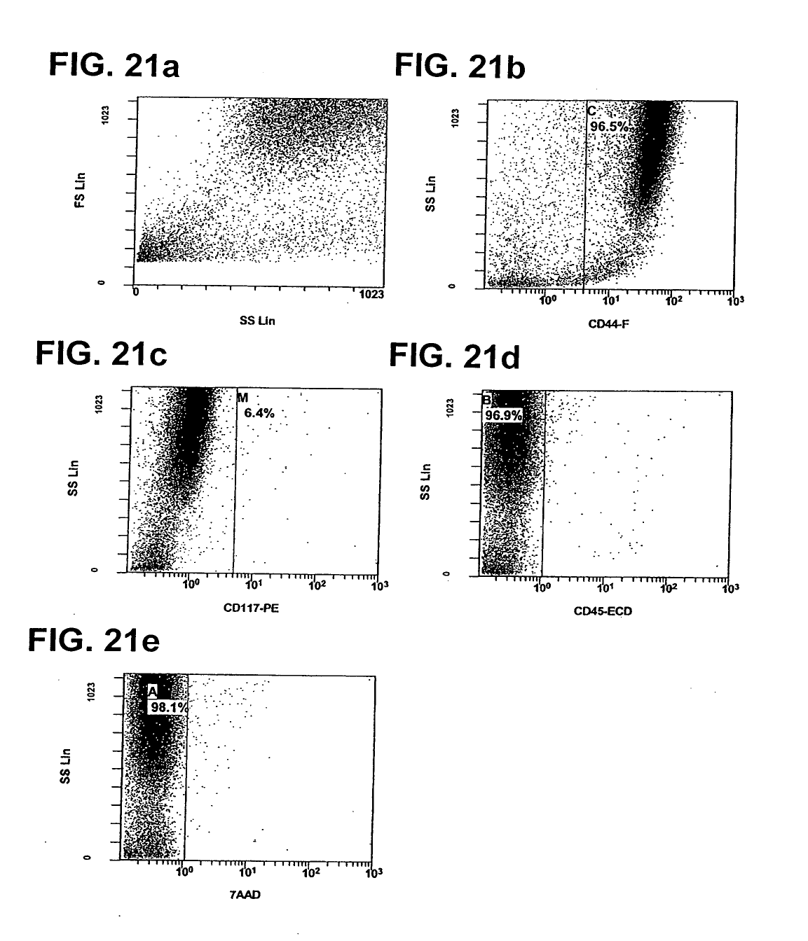

Phenotyping and validity assessment was performed in accordance with the methods described herein for the cells of Cell Line PLE04. Cell counts during cell culture were performed using a hemocytometer. As summarized in Table 13, the data collected from the assessment showed that the cells of Cell Line PLE04 expressed CD44 and CD117 and had low or no expression of CD45 with a high percentage of viability at various passages throughout the duration of the cell culture. The results of the phenotyping and validity assessment are shown in FIGS. 21a through 21e.

TABLE 13
Phenotype and Validity Analysis of Cell Line PLE04

| | | CD44- | CD45- | | |
|---|---|---|---|---|---|
| | CD117- | FITC | ECD | 7AAD- | 7AAD- |
| Passage # | PE | (POS) | (NEG) | TEST | ISO |
| PLE04B - P9 | 5.80% | 96.00% | 96.90% | 98.10% | 97.80% |

Cell Culture for Cell Line PLE05

About 1,000,000 cells comprising maternal placental stem cells obtained from the population of cells suspended in Chang's media were seeded at about 40,000 cells/cm² into a T25 untreated tissue culture flask in 15% Chang's complete media. The cells are associated with those cells collected from the piece of placenta associated with Cell Line PLE05. Fresh 15% Chang's complete media was used through the several passages the cell culture. Flasks were incubated in about 5% CO₂ at about 37° C. temperature.

At passage 1, the harvested cells were transferred to a T75 untreated tissue culture flask. At passages 2 through 8, harvested cells were split into separate cell cultures in T75 untreated tissue culture flasks. At Passage 9, harvested cells were selected for CD117 in accordance with the methodologies of the present invention and as described hereinafter using an MS Column (Miltenyi Biotec). The positive fraction from the CD117 cell selection were analyzed with a hemocytometer to obtain the total number of viable cells and for cell phenotype. The cells in the positive fraction expressed CD44 and CD117 positive and had low or no expression of CD45 with a high percentage of viability. The cells in the positive fraction were cultured further for 15 days in Chang's complete media in untreated tissue culture flasks. The cells in the negative fraction were cultured for 5 days in Chang's complete media in untreated tissue culture flasks.

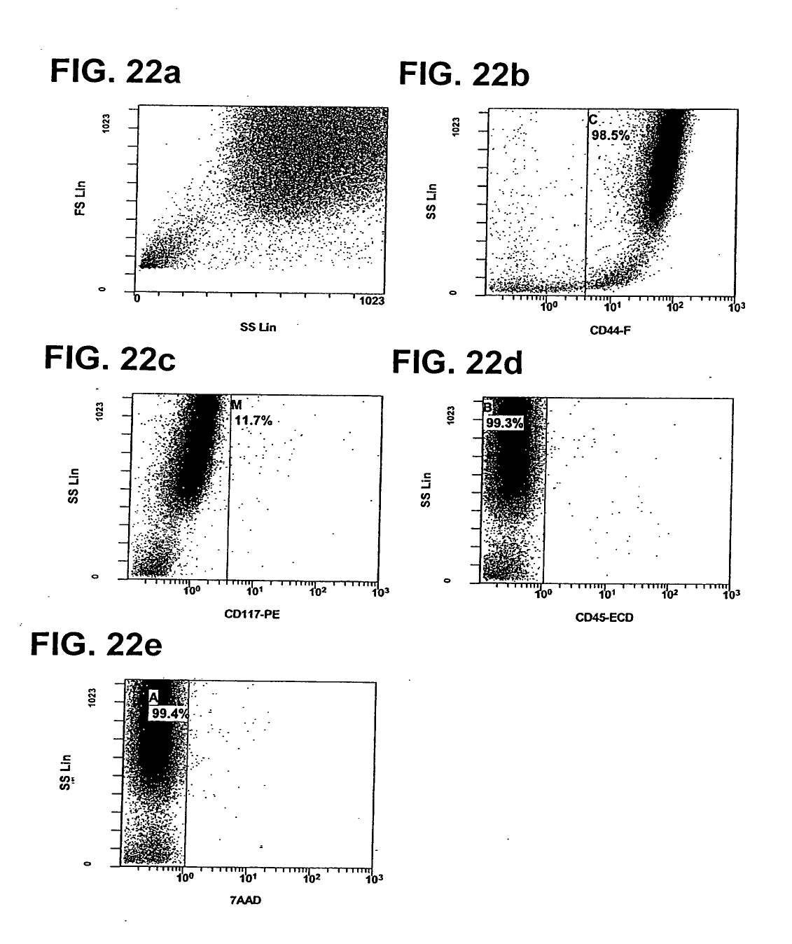

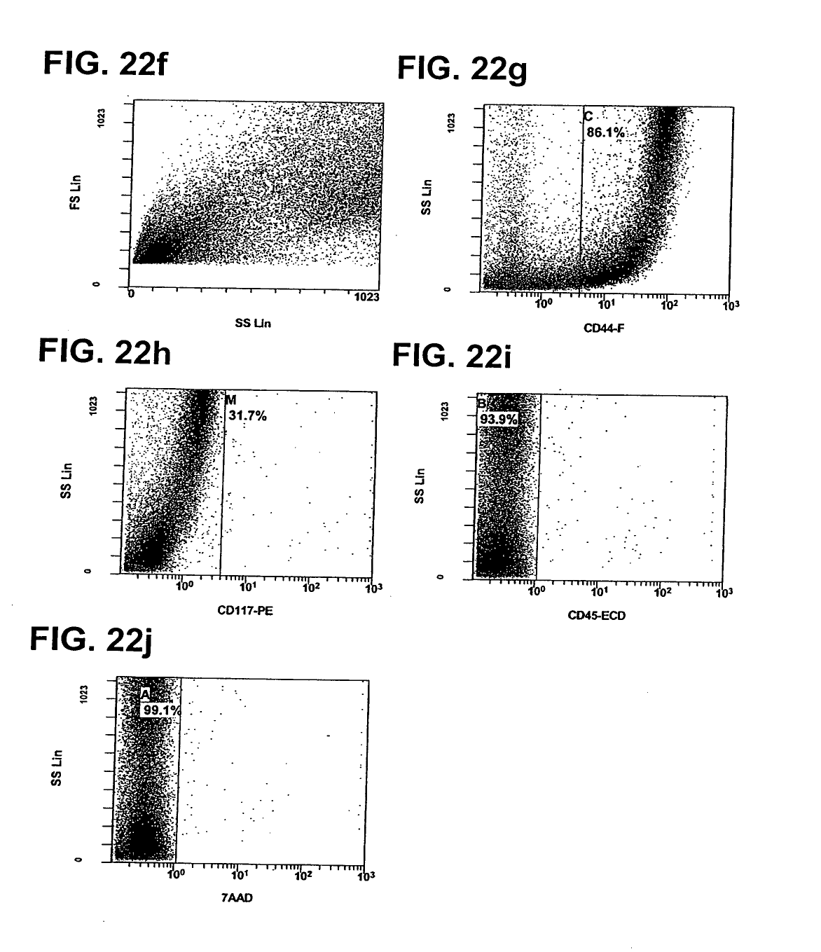

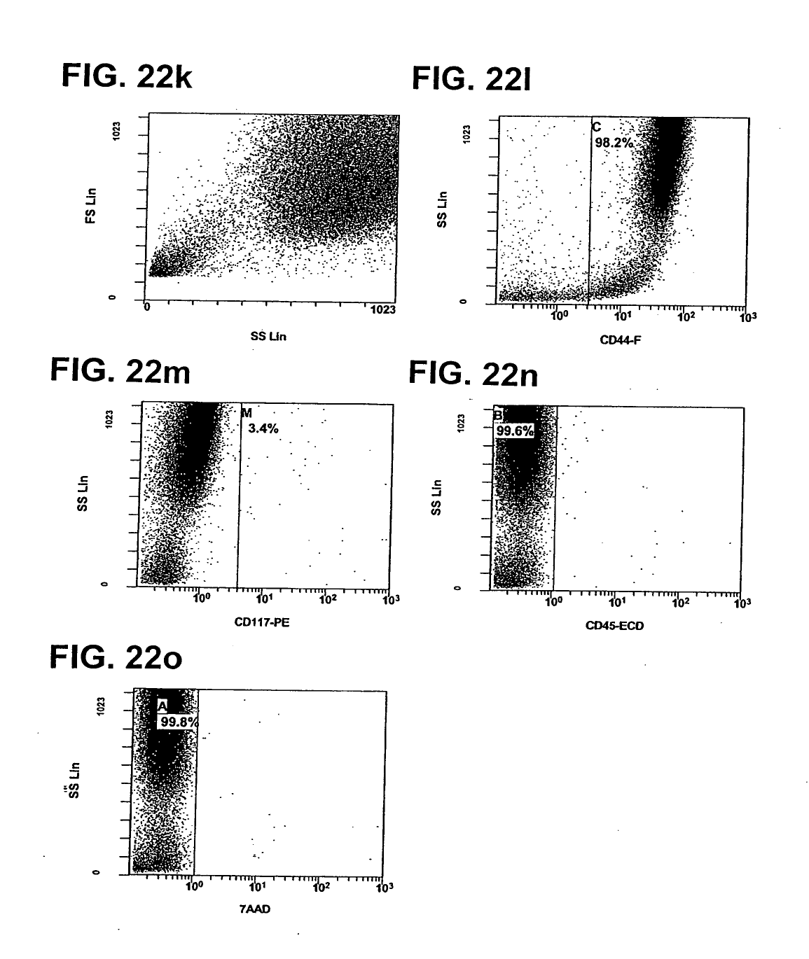

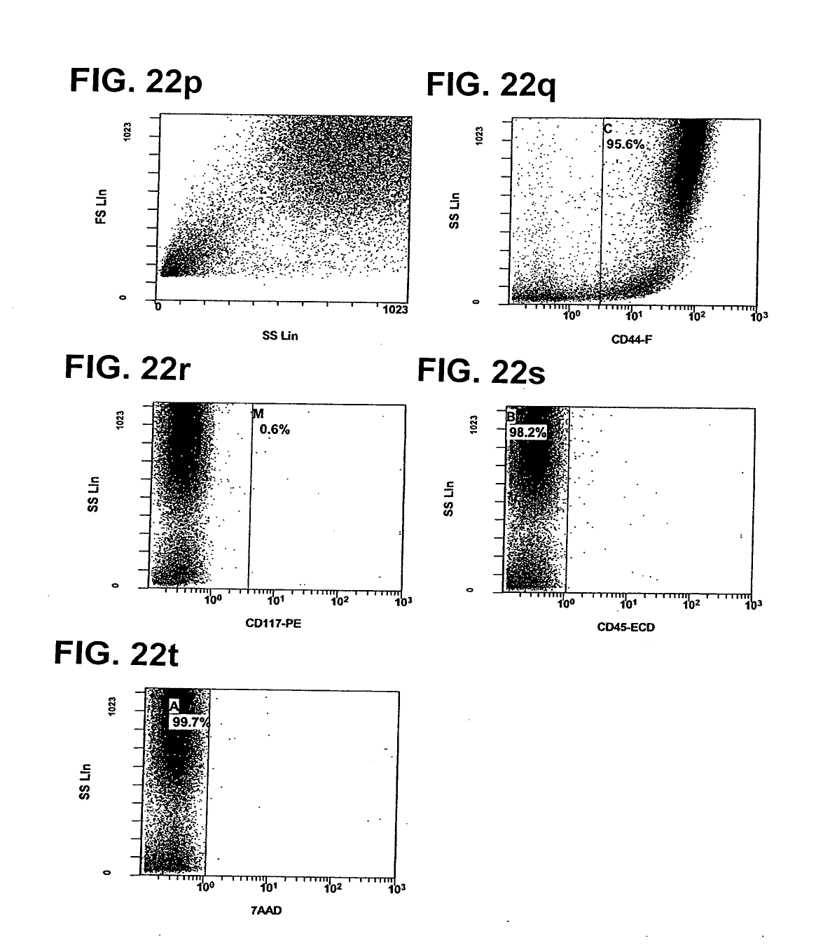

Phenotyping and validity assessment was performed in accordance with the methods described herein for the cells of Cell Line PLE05. Cell counts during cell culture were performed using a hemocytometer. As summarized on Table 14, the data collected from the assessment showed that the cells of Cell Line PLE05 expressed CD44 and CD117 positive and had low or no expression of CD45 with a high percentage of viability at various days throughout the duration of the cell culture. The results of the phenotyping and validity assessment are shown in FIGS. 22a through 22t.

TABLE 14
Phenotype and Validity Analysis of Cell Line PLE05

| | | CD44- | CD45- | | |
|---|---|---|---|---|---|
| | CD117- | FITC | ECD | 7AAD- | 7AAD- |
| Passage # | PE | (POS) | (NEG) | TEST | ISO |
| PLE05B - P9 | 11.40% | 98.30% | 99.30% | 99.40% | 99.20% |
| PLE05B - P1 | 30.90% | 85.70% | 93.90% | 99.10% | 98.50% |
| POS FRAC | | | | | |
| PLE05B - P5 | 3.30% | 98.10% | 99.60% | 99.80% | 99.80% |
| POS PRE (before | | | | | |
| X2 selection) | | | | | |
| PLE05B - P0 | 9.90% | 97.40% | 97.70% | 99.20% | 99.10% |
| POS FRAC (x2) | | | | | |
| PLE05B - P2 | 0.20% | 95.20% | 98.20% | 99.70% | 99.60% |
| (X2) | | | | | |

Cell Culture for Cell Line PLE06

About 1,000,000 cells comprising maternal placental stem cells obtained from the population of cells suspended in Chang's media were seeded at about 40,000 cells/cm² into a T25 untreated tissue culture flask in 15% Chang's complete media. The cells are associated with those cells collected from the piece of placenta associated with Cell Line PLE06. Fresh 15% Chang's complete media was used through the several passages the cell culture. Flasks were incubated in about 5% CO₂ at about 37° C. temperature.

At passages 1 through 7, harvested cells were split into separate cell cultures in T75 untreated tissue culture flasks. At Passage 8, harvested cells were selected for CD117 in accordance with the methodologies of the present invention and as described hereinafter using an MS Column (Miltenyi Biotec). The positive fraction from the CD117 cell selection were analyzed with a hemocytometer to obtain the total number of viable cells and for cell phenotype. The cells at passage 9 and in the positive fraction were CD44 and CD117 positive and CD45 negative and had high percentage of viability. The phenotyping and viability data are provided herewith in Appendix J. The cells in the positive fraction were cultured further for 41 days in Chang's complete media in untreated tissue culture flasks. The cells in the negative fraction were cultured for 7 days in Chang's complete media in untreated tissue culture flasks.

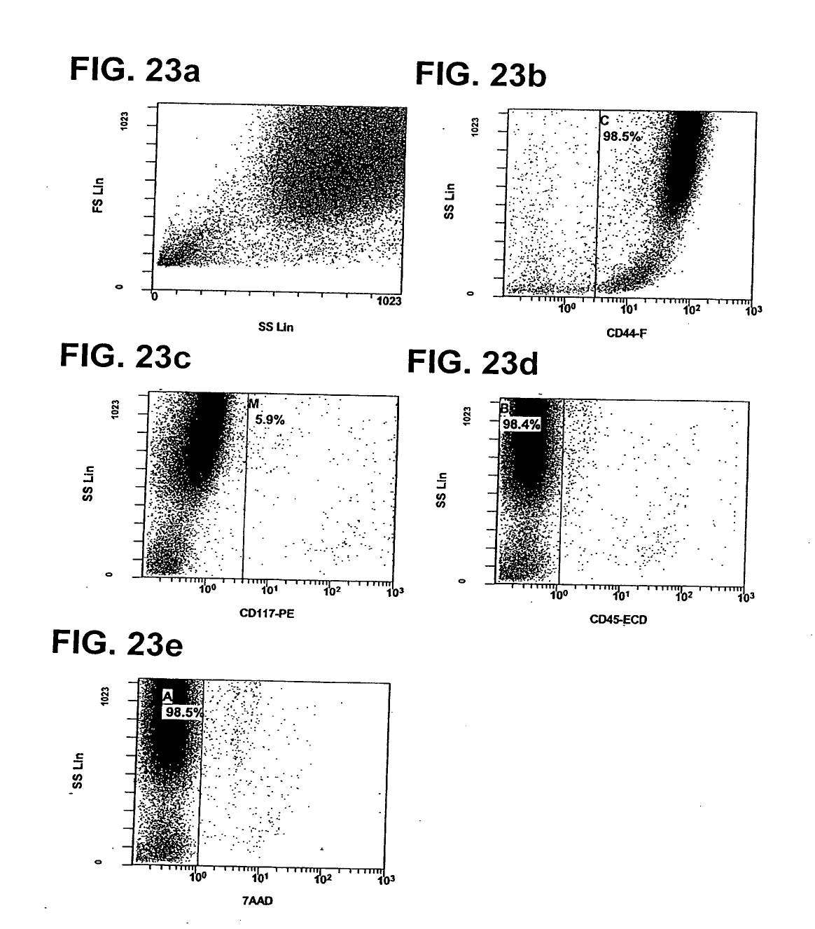

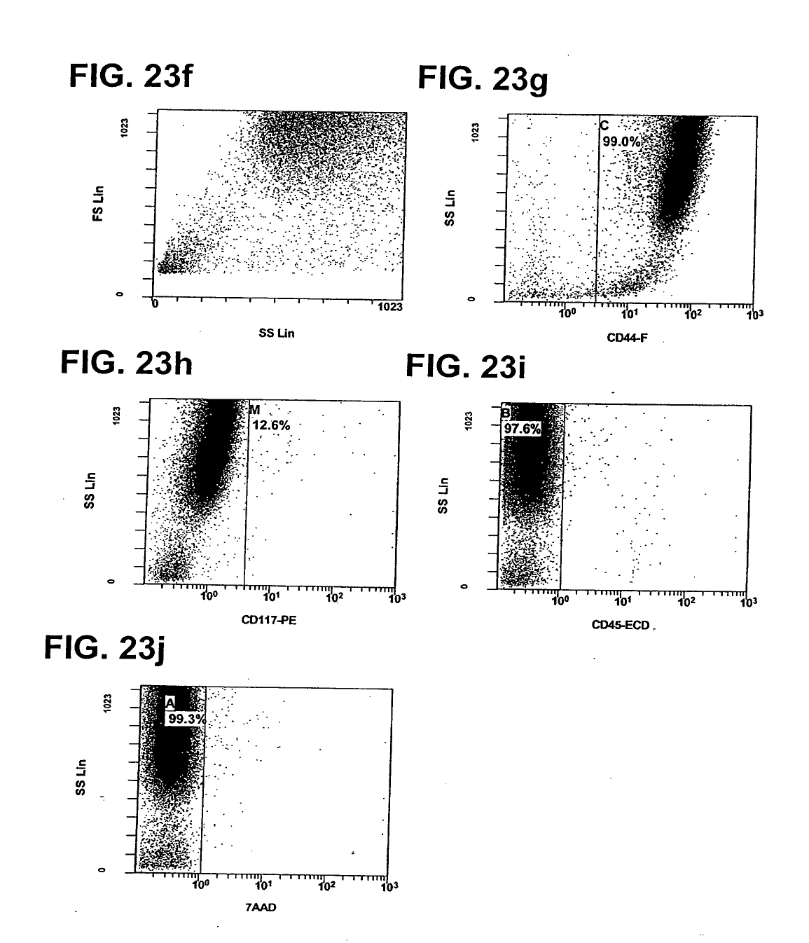

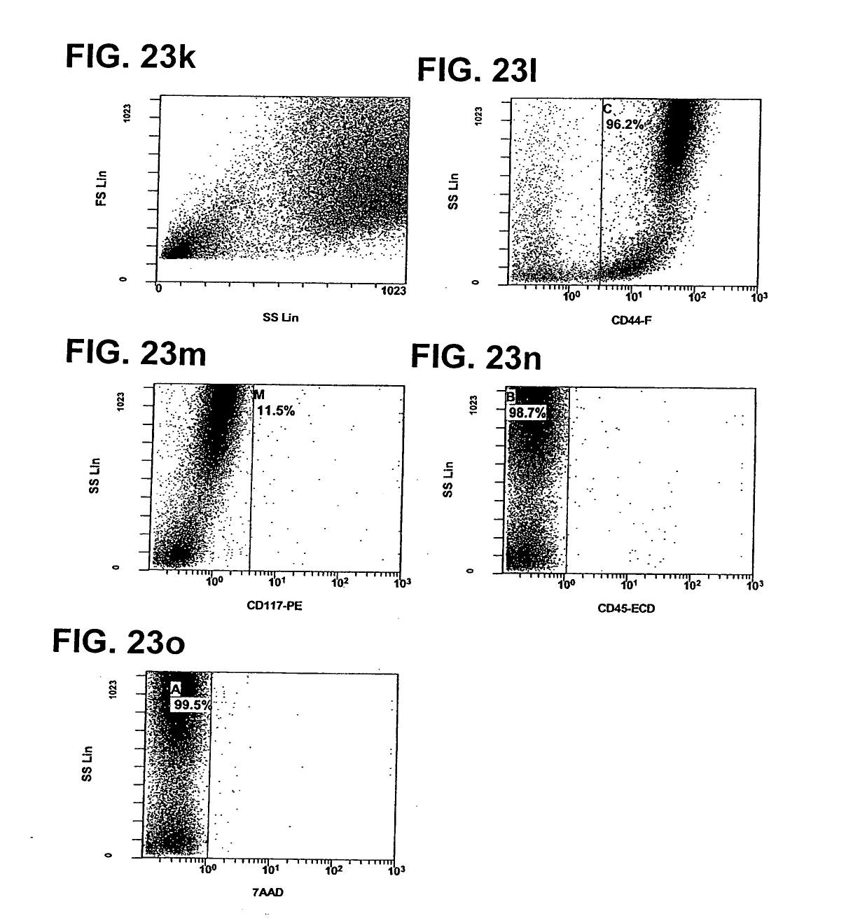

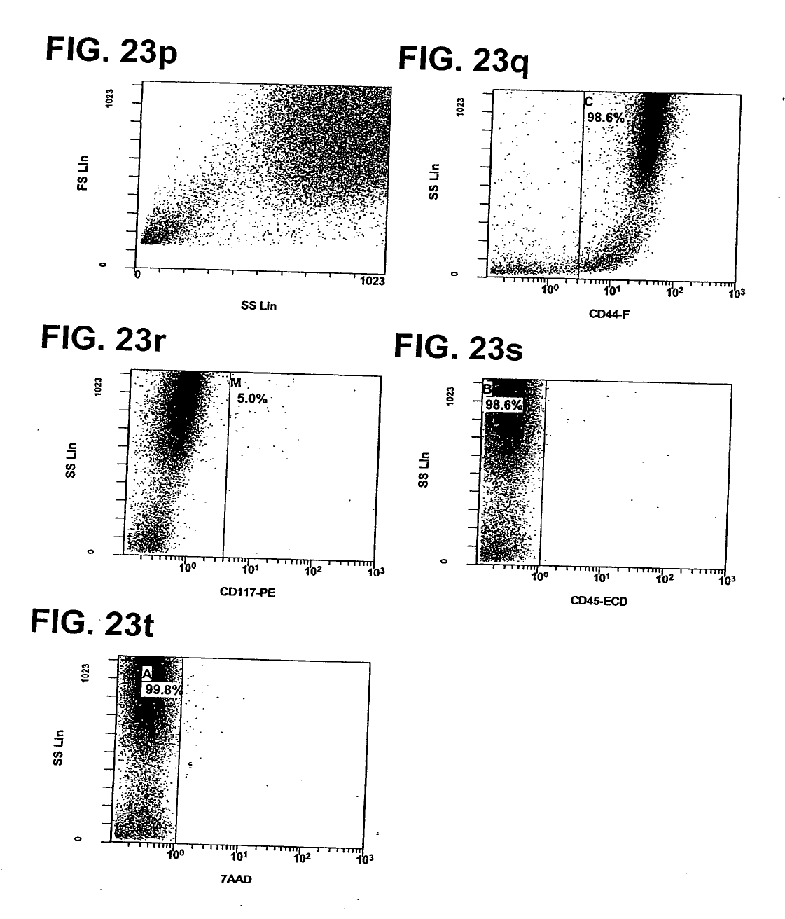

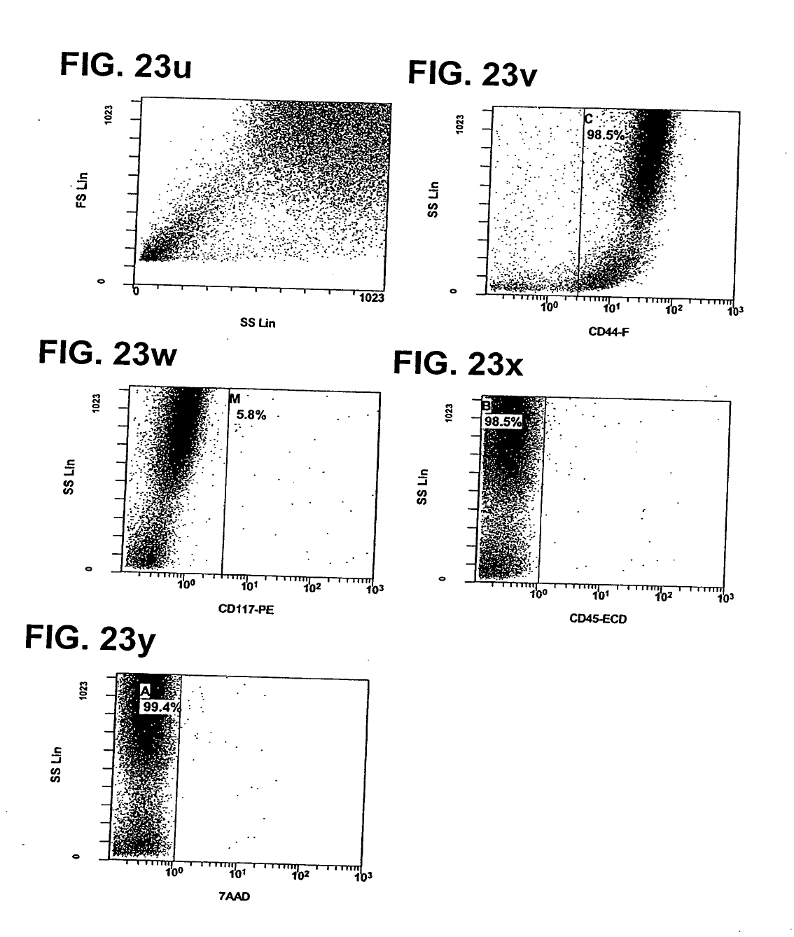

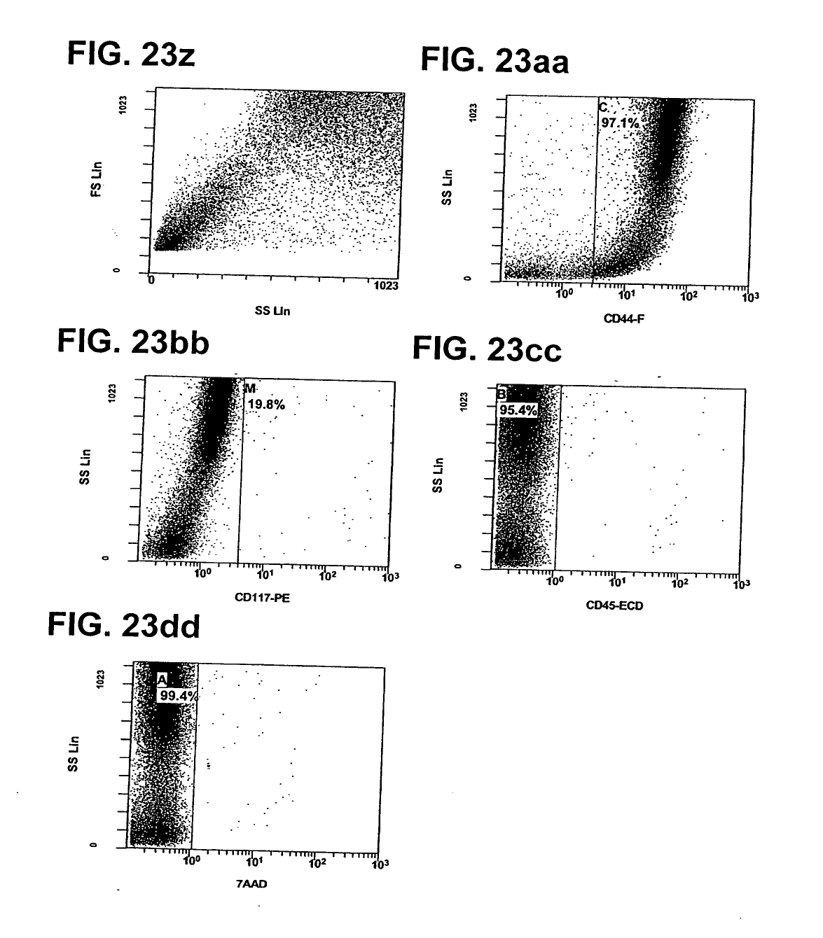

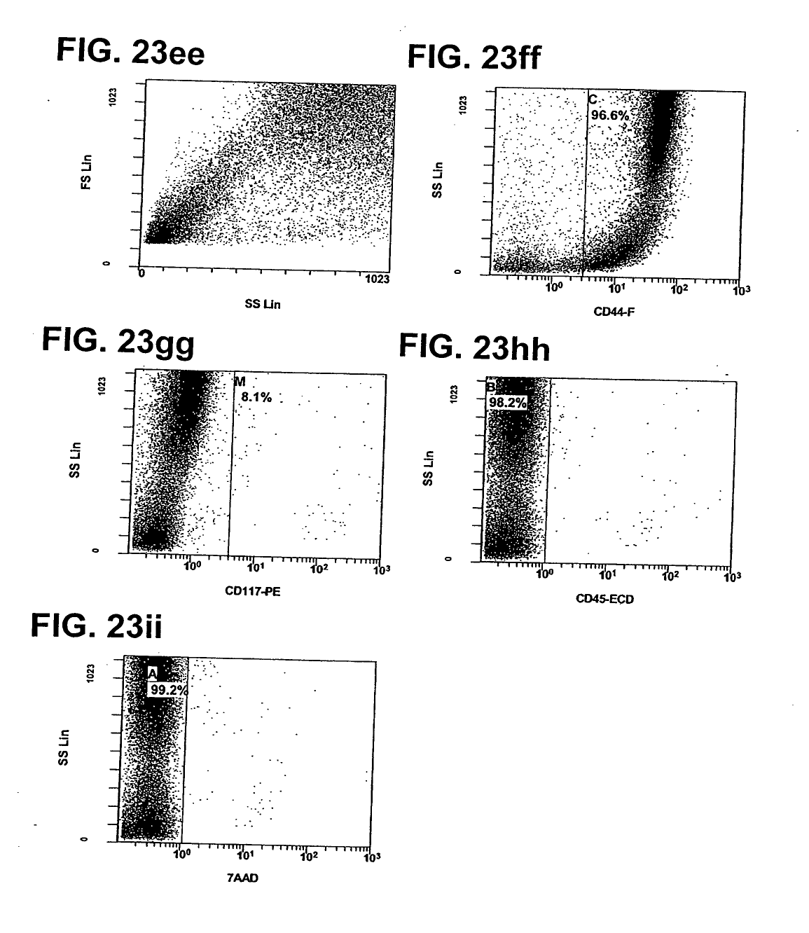

Phenotyping and validity assessment was performed in accordance with the methods described herein for the cells of Cell Line PLE06 throughout the passages of the culture. Cell counts during cell culture were performed using a hemocytometer. As summarized in Table 15, the data collected from the assessment showed that the cells of Cell Line PLE06 expressed CD44 and CD117 and had low or no expression of CD45 with a high percentage of viability at various passages throughout the duration of the cell culture. The results of the phenotyping and validity assessment are shown in FIGS. 23a through 23ii.

TABLE 15
Phenotype and Validity Analysis of Cell Line PLE06

| | | CD44- | CD45- |
|---|---|---|---|
| | CD117- | FITC | ECD |
| Passage # | PE | (POS) | (NEG) |
| PLE06A - P6 | 5.70% | 98.30% | 98.40% |
| PLE06A - P8 | 12.30% | 98.70% | 97.60% |
| PLE06A - P2 | 11.10% | 95.90% | 98.70% |
| POS FRAC | | | |
| PLE06A - P5 | 4.80% | 98.40% | 98.60% |
| POS FRAC | | | |
| PLE06A - P6 | 5.40% | 98.10% | 98.50% |
| POS FRAC | | | |
| PLE06A - P8 | 19.30% | 96.60% | 95.40% |
| POS FRAC | | | |
| PLE06A - P9 | 7.60% | 96.10% | 98.20% |
| POS FRAC | | | |

CD117 Cell Selection for Cell Culture

The cells comprising maternal placental stem cells present in the different cell cultures corresponding with each of Cell Lines PLE02, PLE03, PLE04, PLE05 and PLE06 were separately subjected to CD117 stem cell selection in accordance with the methodologies of the present invention and as described herein. Prior to CD117 stem cell selection, an aliquot of each cell culture was removed for analysis.

The harvested cells were labeled with mouse anti-human CD-117 antibodies. About ten million harvested cells were centrifuged and the supernatant was aspirated. The pellet was suspended in about 100 ul of the working buffer that contained about 5 ug of purified mouse anti-human CD117 monoclonal antibody (IgG₁) with product 104D2 (Santa Cruz). The working buffer available through BD Biosciences contains PBS at about pH of 7.2, bovine serum albumin, EDTA and about 0.09% Azide. The solution containing the cells and antibodies was incubated for about 20 minutes to about 25 minutes on ice. The cells were washed with working buffer to remove unbound antibodies, and the solution containing the cells and antibodies were centrifuged at about 300 g for about 10 minutes. After centrifugation, the supernatant was aspirated and discarded, and the pellet was suspended in about 80 ul of working buffer.

The CD117 stem cell-antibody complexes were labeled with goat anti-mouse antibodies capable of binding to the heavy chain and/or the light chain of mouse IgG. Magnetic microbeads are attached to the goat anti-mouse antibodies. The goat anti-mouse IgG antibodies were added and incubated on ice for about 30 minutes to about 35 minutes. After incubation the cells were washed with about 2 ml of working buffer and centrifuged at about 300 g for about 10 minutes. The supernatant was aspirated and the final cells were suspended in about 500 ul of working buffer.

The cell suspension comprising harvested stem cells and solutions, including working buffer, was kept cold throughout the experiment to prevent non-specific labeling of cells.

An MS Column (Miltenyi Biotec) was prepared by rinsing about 500 ul of working buffer through the column. Minimacs available through Miltenyi Biotec was used for CD117 placental cell selection. The column was placed in the magnetic field of the MACS separator available through Miltenyi Biotec. Each cell suspension comprising maternal placental stem cells was added to the column via pipette. The unlabeled cells and working solution flow through the column and were collected in a sterile tube for phenotyping and cell count analysis. The tube with the collected effluent was labeled as a negative CD117 cell fraction. The column was washed 3 times with working buffer after adding the cells suspension. A sterile tube was placed under the column, which was removed from the magnetic field to allow the positive fraction to pass through the column and into the sterile tube. About one ml of working buffer was added to the column, and a plunger was pushed through column to release the positive fraction of the cells. The tube with the collected effluent was labeled as a positive CD117 cell fraction.

The positive and negative fractions were analyzed with a hemocytometer to obtain the total number of viable cells. The negative fraction was analyzed by flow cytometry.

Genotype Analysis of Cell Lines PLE02 through PLE06

Human Identification-Multiplex Short Tandem Repeat (STR) Analysis was performed on the five cell lines PLE02, PLE03, PLE04, PLE05, and PLE06.

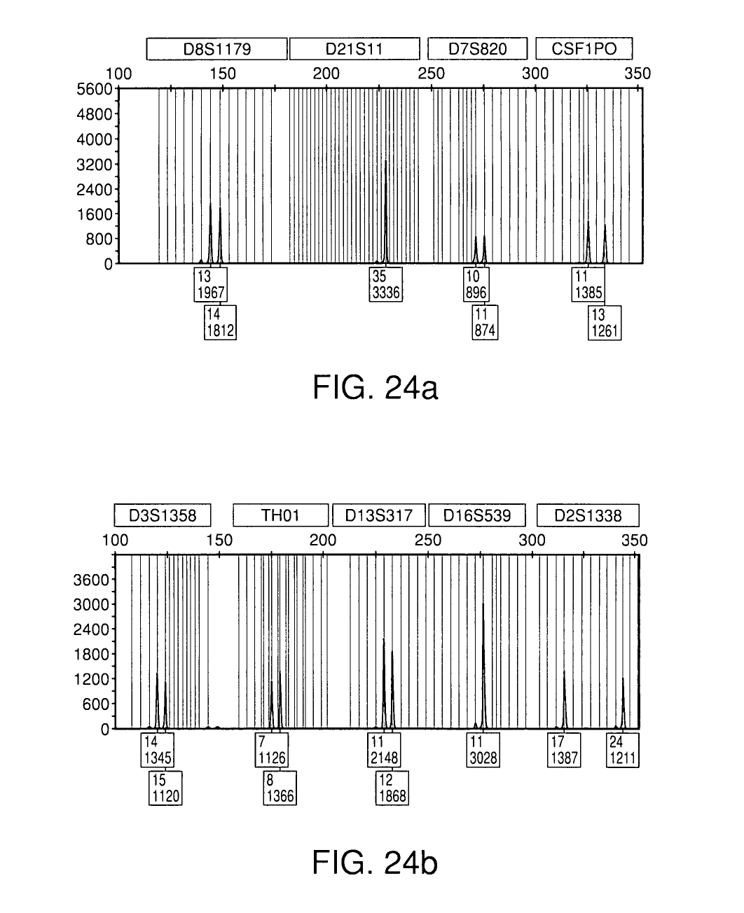

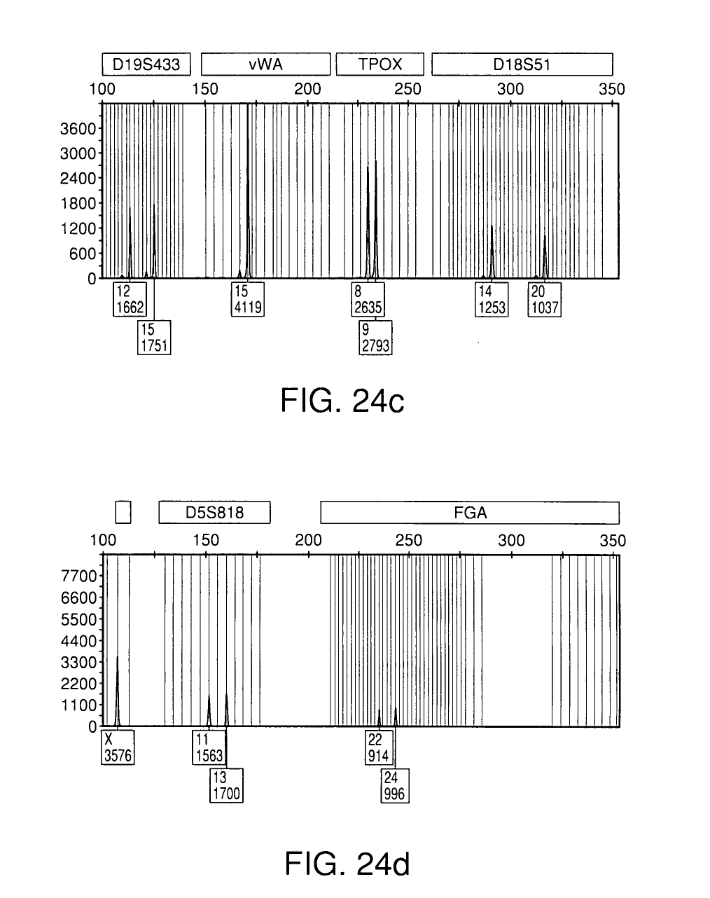

The STR Analysis involved investigating 15 different short tandem repeat (STR) gene regions plus amelogenin on the X and Y chromosomes were simultaneously subjected to PCR and then analyzed. Four separate fluorescent dye labels were used to label the samples. The dyes were coupled to PCR primers. Each of these fluorescent dyes emitted its maximum fluorescence at a different wavelength, that was detected by the 3100. The 15 STR loci investigated were D8S1179, D21S11, D7S820, CSF1P0, D3S1358, TH01, D13S317, D16S539, D2S1338, D19S433, vWA, TPOX, D18S51, D5S818, FGA. The amplified product was electrophoresed on ABI 3100 Genetic Analyzer and analyzed using the GeneMapper ID software program. The sensitivity of the assay to detect mixed chimerism was about 5%. The Cell Lines PLE02 through PLE06 were obtained from placentae for pregnancies in which the child was male. The results of the STR analysis were as follows: the PLE02 specimen is of single individual origin and 100% female and 0% male; the PLE03 specimen is of single individual origin and 100% female and 0% male; the PLE04 specimen is a mixed chimera and 100% female and 0% male; the PLE05 specimen is of a single origin and 100% female and 0% male; and the PLE06 specimen is of single individual origin and 100% female and 0% male. In summary, the results of the STR analysis indicated that all of the cells in Cell Lines PLE02 through PLE06 are maternal as shown in FIGS. 24 through 28.

Phenotype analysis of Cell Lines PLE02 through PLE06 was performed for cell surface markers for stage specific embryonic antigen-4 (SSEA-4), a common marker in embryonic stem cells, and CD73 (ecto-5′-nuclease) and CD105 (endoglin), common markers generally present in mesenchymal and adult stem cells. Further phenotype analysis was performed for the cell surface markers CD45 (leukocyte common antigen), commonly found on hematopoietic cells, and CD133, commonly found on stem cells and progenitor cells from an adult stem cell source. As summarized in Table 16, the cell phenotyping was performed with commercially-available monoclonal antibodies specific for the aforementioned cell surface markers using flow cytometry methods.

TABLE 16
Cell Phenotype Analysis

| Marker/ | | | | | |
|---|---|---|---|---|---|
| Cell line | SSEA-4 | CD73 | CD105 | CD133 | CD45 |
| PLE02 | 16% | 97% | 93% | 2% | 1% |
| PLE03 | 13.5% | 98% | 97% | 2% | 0% |
| PLE04 | 2% | 95% | 94% | 2% | 0% |
| PLE05 | 2% | 97% | 93% | 1% | 0% |
| PLE06 | 2% | 97% | 96% | 1% | 0% |

All five of Cell Lines PLE02 through PLE06 were positive with a high percentage of the cell markers CD73 and CD105. A low percentage of the cells in each cell line expressed CD133. A high percentage of the cells in each cell line expressed low or no CD45. A low percentage of the cells in each cell line were positive for SSEA-4.

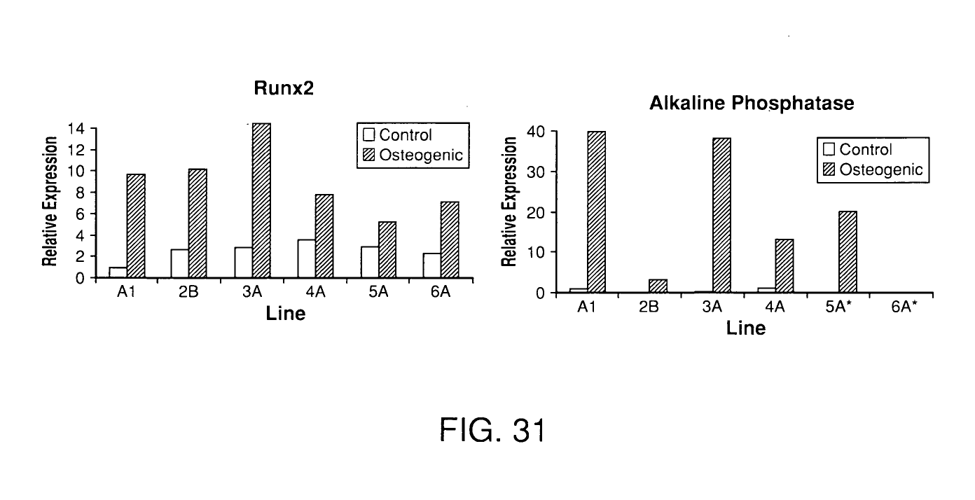

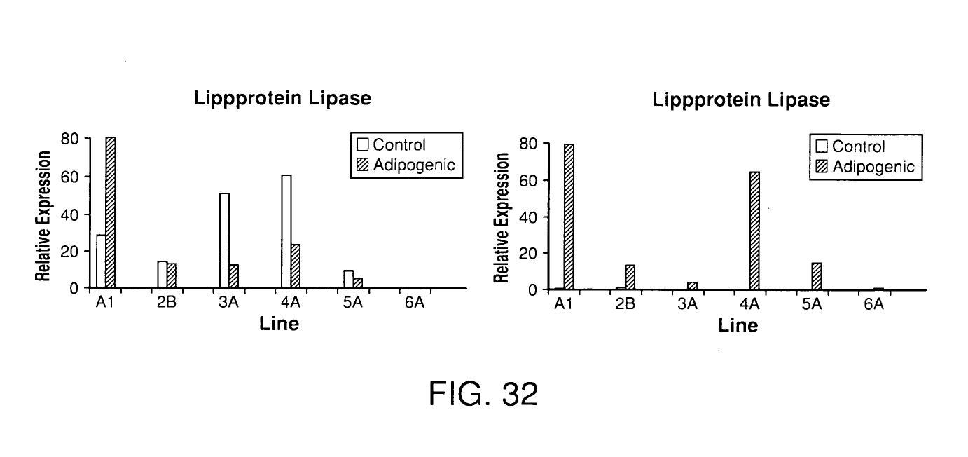

All of the Cell Lines PLE02 through PLE06 were tested for capability to differentiation into three cell lineages including osteogenic, adipogenic and neural cell lineages as shown in FIGS. 30a through 32 using methods in De Coppi et al., Isolation of Amniotic Stem Cells with Potential for Therapy, Nat. Biotechnol. 2007 Jan. 25(1): 100-6. A control for the study was a sample of amniotic fluid cell line A1. All Cell Lines PLE02 through PLE06 expressed Nestin, which demonstrates that the cells were inducted to differentiate into a neural lineage, as shown in FIG. 30a. All Cell Lines PLE02 through PLE06 were positive for Oil-red-0, which demonstrates that the cells were inducted to differentiate into the adipogenic lineage, as shown in FIG. 30c. All of Cell Lines PLE02 through PLE06, except PLE02, were positive for Alizarin Red, which demonstrates that the cells were induced to differentiate into osteoblasts or an osteogenic cell lineage, as shown in FIG. 30b. All of Cell Lines PLE02 through PLE06 demonstrated expression of Cbfa1 with Q-PCR analysis, which is a marker for osetoblastic cell lineage as shown in FIG. 31. All of the Cell Lines PLE02 through PLE06 demonstrated expression of lipoprotein lipase, as shown in FIG. 32. The aforementioned cell differentiation characteristics are summarized in the following Table 17.

TABLE 17
Cell Differentiation

| Cell lineage/ | | | |
|---|---|---|---|
| Cell line | Neurogenic | Osteogenic | Adipogenic |
| A1 | +++ | +++ | +++ |
| PLE-02B | +++ | +/− | +++ |
| PLE-03A | +++ | +++ | +++ |
| PLE-04A | +++ | +++ | ++ |
| PLE-05A | +++ | ++ | +++ |
| PLE-06A | ++ | + | +++ |

### DEFINITIONS

As used herein, the following terms shall have the definitions set forth below, unless the context in which such term is used suggests otherwise.

Amphotericin B (X-Gen) can be obtained at 50 mg/vial (Cardinal-#119140).

BSC means biological safety cabinet.

Cefazolin can be obtained at 1 gm/vial (Cardinal-#3455268).

Cm means centimeter.

Collagenase is an enzyme used to degrade collagen derived from Clostridium histolyticum.

CVS means Chorionic Villus Sampling is generally procured by a health care provider when they insert a small tube in through the vagina or abdomen to remove a small section of chorionic villi tissue from the placenta for prenatal diagnosis and karyotyping.

DMEM means Dulbecco's Minimal Essential Medium.

DMSO means Dimethyl sulfoxide.

DNase means Deoxyribonuclease used to break down DNA found after non-viable cells have lysed.

DPBS means Dulbecco's Phosphate Buffered Saline.

HBSS means Hank's balanced salt solution.

Heparin is a glycosaminoglycan having anticoagulant properties.

HSA means Human Serum Albumin which is an abundant plasma protein that can act as a transporter protein.

IPA means Isopropyl Alcohol used for disinfection typically at about 70% concentration.

LSM means Lymphocyte Separation Media used to perform a density gradient cell separation.

μg means microgram.

μl, μL, ul and uL are used synonymously to mean microliter.

ml and mL are used synonymously to mean milliliter.

QC means Quality Control.

Streptomycin (X-Gen) can be obtained at 1 gm/vial (10) (Cardinal-#2833010).

X means multiply, i.e., by concentration or dilution.

Materials and Equipment

Materials for placental tissue collection kit may include, but are not limited to, placental tissue transport container; sterile tissue container—1 liter; Dulbecco's Phosphate Buffer Saline (DPBS); plastic zipped bags with absorbent towels; sterile scalpel and forceps; sterile ruler; tincture of Iodine and sterile gauze.

Processing materials for placental procurement by scalpel and forceps may include, but are not limited to, sterile scalpel; sterile disposable forceps; tincture of Iodine; DPBS (Mediatech or other suitable source) contains no calcium, magnesium or phenol red; sterile basin; sterile disposable gloves; sterile 4×4 gauze; sterile specimen container; and sterile gloves.

Processing materials for placental procurement by punch biopsy may include, but are not limited to, sterile punch biopsy (8 mm); sterile disposable forceps; tincture of Iodine; DPBS (Mediatech or other suitable source) contains no calcium, magnesium or phenol red; sterile basin; sterile disposable gloves; sterile 4×4 gauze; sterile specimen container; and sterile gloves.

Tissue disinfecting materials may include, but are not limited to, DPBS (Mediatech or other suitable source) contains no calcium, magnesium or phenol red. —1×500 ml bottle; HBSS —1×500 ml bottle; 16-20 g luer lock needles—8; syringe—8; Cavicide; forceps—2; scissors—1; sterile disposable containers—4; blue ice pan; ice; disposable dipping containers—3; sterile disposable gloves; 4×4 gauze—1 package of autoclaved gauze; vacuum collection flask with associated tubing set; red biohazard bags in container; red biohazard sharps container; Betadine hospital grade 10%; Cefazolin, 1 gm/vial (Cardinal-#3455268 or other suitable source)—1 vial; Amphotericin B—X-Gen, 50 mg/vial (Cardinal-#119140 or other suitable source)—2 vials; Streptomycin —X-Gen, 1 gm/vial (10) (Cardinal-#2833010 or other suitable source)—2 vials; and IPA—isopropyl alcohol used for disinfection typically at about 70% concentration.

Tissue disinfecting equipment may include, but is not limited to, a biological safety cabinet (BSC) and automated pipettor.

Materials for placental cell isolation by enzyme digestion may include, but are not limited to, DPBS (Mediatech or other suitable source) contains no calcium, magnesium or phenol red; DNase, Pulmozyme (Genentech, Inc.); Heparin-preservative-free (American Pharmaceutical Partners Inc.) concentration 1000 Units per ml; Collagenase contains Class I and II—(Serva/Cresent Chemical)—either 500 mg NB-4 for research (cat# 17454.02) or 1 gram NB6 for GMP use (cat# 17458.01). Both may contain the same PZ activity > or =0.1 U/mg lyophilysate; Human Serum Albumin, 25% (Baxter healthcare Corporation, Glendale, Calif., USA or other suitable source); Cavicide; 70% Isopropyl alcohol; scissors; forceps; disposable scalpel; 50 ml tube rack; 5 ml tube rack; cell strainer—100 micron filter (BD); centrifuge inserts; blue ice pan; ice; alcohol wipes, about 70% Isopropyl alcohol; red top vacutainer tubes—5 ml; BacT/ALERT blood culture bottle; sterile 50 ml conicals; sterile Petri dish; 1 ml needle TB Syringe; 3 ml needle syringe; 16-20 g luer lock needles; 10 ml sterile pipette; sterile aspirating pipettes; 3 sterile transfer pipettes; sterile disposable gloves; 4×4 gauze; vacuum collection flask with associated tubing set; red biohazard bags in container; red biohazard sharps container; and specimen labels.

Equipment for placental cell isolation by enzyme digestion may include, but is not limited to, centrifuge with round buckets; centrifuge Inserts; biological safety cabinet (BSC); vacuum pump; inverted light microscope; scale; hemocytometer; 37° C. incubator; and automated pipettor.

Materials for placental cell isolation by mechanical separation may include, but are not limited to, DPBS (Mediatech) contains no calcium, magnesium or phenol red; Lymphocyte Separation Media (Mediatech cat. #25-072-CV); DNase, Pulmozyme (Genentech, Inc.); Heparin-preservative-free (American Pharmaceutical Partners Inc.) concentration 1000 units per ml; Human Serum Albumin, 25% (Baxter Healthcare Corporation, Glendale, Calif., USA or other suitable source); Cavicide; 70% Isopropyl alcohol; scissors; forceps; disposable scalpel; 50 ml tube rack; 5 ml tube rack; centrifuge inserts; cell strainer—100 micron filter (BD); blue ice pan; Ice; alcohol wipes, 70% Isopropyl alcohol; red top vacutainer tubes—5 ml; BacT/ALERT blood culture bottle; sterile 50 ml conicals; sterile Petri dish; 1 ml needle TB syringe; 3 ml needle syringe; 16-20 g luer lock needles; 10 ml sterile pipette; 3 sterile transfer pipettes; sterile disposable gloves; 4×4 gauze; vacuum collection flask with associated tubing set; red biohazard bags in container; red biohazard sharps container; and specimen labels.

Equipment for placental cell isolation by mechanical separation may include, but is not limited to, centrifuge with round buckets; centrifuge inserts; biological safety cabinet (BSC); vacuum pump; inverted light microscope; scale; and automated pipettor.

Processing materials may include, but are not limited to, DPBS (Mediatech or other suitable source) contains no calcium, magnesium or phenol red. —2×500 ml bottles; Lymphocyte Separation Media (Mediatech cat. #25-072-CV)-1×500 ml bottle; DNase, Pulmozyme (Genentech Inc.)—1×2.5 ml vial; Heparin-preservative-free (American Pharmaceutical Partners Inc) concentration 1000 Units per ml)—3×2 ml vials; Human Serum Albumin, 25% (Baxter Healthcare Corporation, Glendale, Calif., USA or other suitable source). —1 bottle; Cavicide; about 70% Isopropyl alcohol; forceps—3; disposable scalpel—1; 50 ml tube rack; 5 ml tube rack; 15 ml tube rack; centrifuge inserts; cell strainer—100 micron filter (BD)—16; blue ice pan; ice; alcohol wipes, about 70% Isopropyl alcohol; red top vacutainer tubes—5 ml×2; BacT/ALERT blood culture bottles; sterile 50 ml conicals—16; sterile 15 ml conicals—4; sterile Petri dish—2; 1 ml needle TB syringe—4; 3 ml needle syringe—4; 16-20 g luer lock needles—4; 10 ml sterile pipette—4; sterile aspirating pipettes—4; 3 sterile transfer pipettes—6; sterile disposable gloves; 4×4 gauze—1 package of autoclaved gauze; vacuum collection flask with associated tubing set; red biohazard bags in container; red biohazard sharps container; sterile steel basin—for sterile supplies; and specimen labels.

Processing equipment may include, but is not limited to, centrifuge with round buckets; centrifuge inserts; biological safety cabinet (BSC); vacuum pump; inverted light microscope; scale; and automated pipettor.

Cryopreservation materials may include, but are not limited to, DPBS; DMSO; 25% Human Serum Albumin; wash solution; bar-coded cryovial—1×5 ml; and QC vials—5×1 ml.

Cryopreservation equipment may include, but are not limited to, Planar cryopreservation freezer and liquid Nitrogen storage freezer.

Materials for flow cytometry may include, but are not limited to, FC500 Flow Cytometer; human placental cells within 48 hours of cell isolation; 5 uL to 100 uL micropipettor; Eppendorf pipettor; manual/electric pipettor; 1-200 uL pipette Tips; Plastibrand positive displacement tips (5.0 ml); serological pipettes, 5 ml and 25 ml; 12×75 mm polypropylene culture tubes; 50 ml tubes; test tube racks; distilled water; Isoflow Sheath fluid—stable at room temperature until expiration date on label, Do Not Freeze; and Coulter Clenz cleaning agent—store between about 2° C. to about 25° C., stable until expiration date or about 3 months after opening, remix by inversion if frozen and thawed.

Reagents for flow cytometry may include, but are not limited to, CD117-PE—stable to expiration date on vial when stored between about 2° C. to about 8° C. away from light, stable about 30 days after opening, watch for evidence of deterioration (change in color and/or clarity), bring to between about 20° C. to about 25° C. before use; CD44-FITC—stable to expiration date on vial when stored between about 2° C. to about 8° C. away from light, stable 30 days after opening, watch for evidence of deterioration (change in color and/or clarity), bring to between about 20° C. to about 25° C. before use; CD45-ECD—stable to expiration date on vial when stored between about 2° C. to about 8° C. away from light, stable 30 days after opening, watch for evidence of deterioration (change in color and/or clarity), bring to between about 20° C. to about 25° C. before use; IgG—FITC—stable to expiration date on vial when stored between about 2° C. to about 8° C. away from light, stable 30 days after opening, watch for evidence of deterioration (change in color and/or clarity), bring to between about 20° C. to about 25° C. before use; IgG—PE—stable to expiration date on vial when stored at between about 2° C. to about 8° C. away from light, stable 30 days after opening, watch for evidence of deterioration (change in color and/or clarity), bring to between about 20° C. to about 25° C. before use; IgG—ECD—stable to expiration date on vial when stored at between about 2° C. to about 8° C. away from light, stable 30 days after opening, watch for evidence of deterioration (change in color and/or clarity), bring to between about 20° C. to about 25° C. before use; 7-AAD viability dye—stable to expiration date on vial when stored between about 2° C. to about 8° C. away from light, stable 30 days after opening, watch for evidence of deterioration (change in color and/or clarity), bring to between about 20° C. to about 25° C. before use; Ammonium Chloride (NJ4CL) lysing solution 10× concentrated, stored at between about 2° C. to about 8° C., stable until expiration date, use working solutions at room temperature, discard at end of day; Human Serum Albumin 25%, store at between about 2° C. to about 8° C.; wash media comprising HBSS (Hanks with Ca+ and Mg+) 100 ml, 0.2 Heparin about 1 ml; HSA 25% about 10 ml; DNase about 20 drops; Kasumi-3 cell line—CD117+ cells; Stemtrol control cells—CD34− cells, stable to expiration date on vial when stored at between about 2° C. to about 8° C. away from light, stable 30 days after opening, watch for evidence of deterioration (change in color and/or clarity), bring to between about 20° C. to about 25° C. before use; timer; and vortex mixer.

The materials for cell culture may include but are not limited to a 37° C. water bath, hemocytometer, cover slips, lens paper, alcohol prep pads, 5 uL to 100 uL micropipettor, Eppendorf pipettor, manual/electric pipettor, 200 uL pipette tips, serological pipettes, 5 ml and 25 ml, 12×75 mm polypropylene culture tubes, 50 ml tubes, test tube racks, and untreated flasks.

Other suitable replacement reagents and products and manufacturers may be used in place of the specific reagents, products and manufacturers listed herein.

Modifications can be made to the embodiments described above without departing from the broad inventive concept thereof. Having described the preferred embodiments of the invention, additional embodiments, adaptations, variations, modifications and equivalent arrangements will be apparent to those skilled in the art. These and other embodiments will be understood to be within the scope of the appended claims and apparent to those skilled in the art.

What is claimed is:

1. A method for obtaining a population of cells comprising maternal placental stem cells, the method comprising:
(a) disaggregating placental tissue and separating the population of cells from disaggregated placental tissue;
(b) collecting and concentrating the population of cells; and
(c) cryopreserving the population of placental cells at or below about −135°.

2. The method of claim 1, wherein the maternal placental stem cells express the cell surface marker CD117 and at least one of the cell surface markers selected from the group consisting of CD29, CD44, CD73, CD90, CD105, CD166, SSEA-3 and SSEA-4 and have low or no expression of at least one of the cell surface markers selected from the group consisting of CD34, CD45 and CD133 from placental tissue.

3. A process for obtaining a population of cells comprising maternal placental stem cells, the method comprising:
(a) procuring placental tissue from a whole placenta, the placental tissue comprising maternal tissue and fetal tissue;
(b) disaggregating the placental tissue;
(c) isolating the population of cells comprising maternal placental cells from disaggregated maternal tissue; and
(d) collecting the population of cells comprising maternal placental stem cells by concentrating the population of placental cells with at least one step of centrifugation.

4. The process of claim 3, wherein the maternal placental stem cells express the cell surface marker CD117 and at least one of the cell surface markers selected from the group consisting of CD29, CD44, CD73, CD90, CD105, CD166, SSEA-3 and SSEA-4 and have low or no expression of at least one of the cell surface markers selected from the group consisting of CD34, CD45 and CD133 from placental tissue.

5. A process for collecting maternal placental stem cells expressing CD117 from placental tissue, the process comprising:
(a) isolating placental cells comprising the maternal placental stem cells from the placental tissue by disaggregating the placental tissue;
(b) collecting and concentrating the placental cells; and
(c) cryopreserving the placental cells.

6. The process of claim 5, wherein the maternal placental stem cells also express at least one of the cell surface markers selected from the group consisting of CD29, CD44, CD73, CD90, CD105, CD166, SSEA-3 and SSEA-4 and have low or no expression of at least one of the cell surface markers selected from the group consisting of CD34, CD45 and CD133 from placental tissue.

7. A system for collecting a population of cells comprising maternal placental stem cells, comprising:
(a) a placental cell isolater, wherein the placental cell isolator disaggregates placental tissue comprising maternal tissue and separates placental cells from the disaggregate placental tissue;
(b) a placental cell collector, wherein the placental cell collector collects the placental cells separated from the disaggregate placental tissue:
(c) a placental cell concentrator, wherein the placental cell concentrator concentrates placental cells present in a suspension; and
(d) a placental cell cryopreserver, wherein the placental cell cryopreserver maintains the collected and concentrated placental cells at a temperature at or below about −135° C.

8. A process for isolating maternal placental stem cells expressing CD117 from a population of placental cells, the process comprising:
(a) culturing a population of placental cells comprising maternal placental stem cells;
(b) selecting placental cells expressing CD117 from a culture of the population of placental cells; and
(c) cryopreserving the maternal placental stem cells expressing CD117.

9. The process of claim 8, wherein the maternal placental stem cells also express at least one of the cell surface markers selected from the group consisting of CD29, CD44, CD73, CD90, CD105, CD166, SSEA-3 and SSEA-4 and have low or no expression of at least one of the cell surface markers selected from the group consisting of CD34, CD45 and CD133 from placental tissue.

10. A process for isolating a population of maternal placental stem cells from a population of placental cells, comprising, selecting maternal placental stem cells expressing CD117 from a culture of the population of placental cells.

11. A process for isolating a population of maternal placental stem cells expressing CD117 from a population of placental cells, the process comprising,
(a) selecting placental cells expressing CD117 from a population of placental cells comprising maternal placental stem cells;
(b) culturing the placental cells expressing CD117 selected from the population of placental cells comprising maternal placental stem cells; and
(c) selecting placental cells expressing CD117 from a culture of placental cells.

12. A population of cells enriched for maternal placental stem cells obtained from the process comprising:
(a) culturing a population of cells comprising maternal placental stem cells;
and
(b) selecting cells expressing CD117 from a culture of the population of cells.

13. A population of maternal placental stem cells obtained from the process comprising selecting placental cells expressing CD117 from a culture of a population of placental cells.

14. The population of maternal placental cells of claim 13, wherein the maternal placental stem cells express the cell surface marker CD117 and at least one of the cell surface markers selected from the group consisting of CD29, CD44, CD73, CD90, CD105, CD166, SSEA-3 and SSEA-4 and have low or no expression of at least one of the cell surface markers selected from the group consisting of CD34, CD45 and CD133 from placental tissue.

15. Maternal placental stem cells obtained from the process comprising
(a) selecting placental cells expressing CD117 from a population of placental cells comprising maternal placental stem cells;
(b) culturing the placental cells expressing CD117 selected from the population of placental cells comprising maternal placental stem cells; and
(c) selecting placental cells expressing CD117 from a culture of placental cells expressing CD117.

16. A population of cells enriched for maternal placental stem cells expressing CD117.

17. A population of cells enriched for maternal placental stem cells express the cell surface marker CD117 and at least one of the cell surface markers selected from the group consisting of CD29, CD44, CD73, CD90, CD105, CD166, SSEA-3 and SSEA-4 and have low or no expression of at least one of the cell surface markers selected from the group consisting of CD34, CD45 and CD133 from placental tissue.

18. A population of cells enriched for maternal placental stem cells expressing CD44 and CD117 and having low or no expression of CD45.

19. A composition comprising a population of cells enriched for maternal placental stem cells and a preservation agent.

20. A composition comprising at least one maternal placental stem cell and a preservation agent.

21. The composition of claim 20, wherein the at least one maternal placental stem cell expresses at least one of the cell surface markers selected from the group consisting of CD29, CD44, CD73, CD90, CD105, CD117, CD166, SSEA-3 and SSEA-4 and has low or no expression of at least one of the cell surface markers selected from the group consisting of CD34, CD45 and CD133 and a preservation agent.

22. At least one maternal placental stem cell obtained from the process comprising:
(a) procuring placental tissue comprising maternal tissue and fetal tissue from a whole placenta;
(b) disaggregating the placental tissue;
(c) isolating placental cells comprising maternal placental stem cells from disaggregated placental tissue;
(d) collecting and concentrating placental cells comprising maternal placental stem cell in a population of cells;
(e) culturing the population of placental cells comprising maternal placental stem cell; and
(f) selecting at least one maternal placental stem cell expressing CD117 from a culture of the population of placental cells.

23. The process of claim 22, wherein the process comprises the further steps of:
(a) culturing at least one maternal placental stem cell expressing CD117 selected from the population of placental cells; and
(b) selecting at least one maternal placental stem cell expressing CD117 from a culture of placental cells expressing CD117 comprising a population of at least one maternal placental stem cell expressing CD117.
